(19) United States
(12) Patent Application Publication
(10) Pub. No.: US 2005/0192261 A1
(43) Pub. Date: Sep. 1, 2005

(54) Methods and reagents for the treatment of immunoinflammatory disorders

(76) Inventors: Edward Roydon Jost-Price, West Roxbury, MA (US); Palaniyandi Manivasakam, West Roxbury, MA (US); Bradley B. Brasher, Natick, MA (US); Brendan Smith, Boston, MA (US); Noah Sachs, Boston, MA (US); Benjamin A. Auspitz, Cambridge, MA (US); Todd W. Chappell, Boston, MA (US); Micheal S. Slavonic, Quincy, MA (US)

Correspondence Address: CLARK & ELBING LLP, 101 FEDERAL STREET, BOSTON, MA, 02110, US

(21) Appl. No.: 10/940,902

(22) Filed: Sep. 14, 2004

Related U.S. Application Data
(60) Provisional application No. 60/503,026, filed on Sep. 15, 2003.

(51) Int. Cl. A61K 31/573; A61K 31/5415; A61K 31/473; A61K 31/4172

(52) U.S. Cl. 514/171; 514/290; 514/225.8; 514/400

(57) ABSTRACT

The invention features a method for treating a patient diagnosed with, or at risk of developing, an immunoinflammatory disorder by administering to the patient an antihistamine, either alone or in combination with one or more additional agents. The invention also features a pharmaceutical composition containing an antihistamine in combination with one or more additional agents.

### CROSS-REFERENCE TO RELATED APPLICATIONS

This application claims benefit from U.S. Provisional application No. 60/503,026, filed on Sep. 15, 2003, hereby incorporated by reference.

### BACKGROUND OF THE INVENTION

The invention relates to the treatment of immunoinflammatory disorders.

Immunoinflammatory conditions are characterized by the inappropriate activation of the body's immune defenses. Rather than targeting infectious invaders, the immune response targets and damages the body's own tissues or transplanted tissues. The tissue targeted by the immune system varies with the disorder. For example, in multiple sclerosis, the immune response is directed against the neuronal tissue, while in Crohn's disease the digestive tract is targeted. Immunoinflammatory disorders affect millions of individuals and include conditions such as asthma, allergic intraocular inflammatory diseases, arthritis, atopic dermatitis, atopic eczema, diabetes, hemolytic anaemia, inflammatory dermatoses, inflammatory bowel or gastrointestinal disorders (e.g., Crohn's disease and ulcerative colitis), multiple sclerosis, myasthenia gravis, pruritis/inflammation, psoriasis, rheumatoid arthritis, cirrhosis, and systemic lupus erythematosus.

Current treatment regimens for immunoinflammatory disorders typically rely on immunosuppressive agents. The effectiveness of these agents can vary and their use is often accompanied by adverse side effects. Thus, improved therapeutic agents and methods for the treatment of immunoinflammatory conditions are needed.

### SUMMARY OF THE INVENTION

The invention features a method for treating an immunoinflammatory disease by administering to a patient in need thereof certain antihistamines, either alone or in combination with any of a number of additional agents.

Accordingly, in one aspect, the invention features a method of treating an immunoinflammatory disease in a patient in need thereof by administering to the patient any one of certain antihistamines in an amount and for a duration to treat the disease.

In another aspect, the invention features a pharmaceutical composition that includes an antihistamine and a corticosteroid. Particularly desirable antihistamines are bromodiphenhydramine, clemizole, cyproheptadine, desloratadine, loratadine, thiethylperazine maleate, and promethazine, while particularly desirable corticosteroids are prednisolone, cortisone, dexamethasone, hydrocortisone, methylprednisolone, fluticasone, prednisone, triamcinolone, and diflorasone. The composition may be formulated for topical, administration, or for systemic administration (e.g., oral administration). One or both of the drugs may be present in the composition in a low dosage or a high dose, each of which is defined herein.

In another aspect, the invention features a.method of decreasing proinflammatory cytokine secretion or production in a patient by administering to the patient an antihistamine and a corticosteroid simultaneously or within 14 days of each other in amounts sufficient to decrease proinflammatory cytokine secretion or production in the patient.

In a related aspect, the invention features a method for treating a patient diagnosed with or at risk of developing an immunoinflammatory disorder by administering to the patient an antihistamine and a corticosteroid simultaneously or within 14 days of each other in amounts sufficient to treat the patient. In another aspect, the invention features a kit that includes: (i) a composition that includes an antihistamine and a corticosteroid; and (ii) instructions for administering the composition to a patient diagnosed with or at risk of developing an immunoinflammatory disorder.

If desired, any of the above methods may include administration of one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator non-steroidal immunophilin-dependent immunosuppressant, vitamin D analog, psoralen, retinoid, or 5-amino salicylic acid.

In a related aspect, the invention features a kit that includes: (i) an antihistamine; (ii) a corticosteroid; and (iii) instructions for administering the antihistamine and the corticosteroid to a patient diagnosed with or at risk of developing an immunoinflammatory disorder.

In another aspect, the invention features a pharmaceutical composition that includes an antihistamine and ibudilast. The composition may be formulated for topical administration, or for systemic administration.

In another aspect, the invention features a method of decreasing proinflammatory cytokine secretion or production in a patient by administering to the patient an antihistamine and ibudilast simultaneously or within 14 days of each other in amounts sufficient to decrease proinflammatory cytokine secretion or production in the patient.

In a related aspect, the invention features a method for treating a patient diagnosed with or at risk of developing an immunoinflammatory disorder by administering to the patient an antihistamine and ibudilast simultaneously or within 14 days of each other in amounts sufficient to treat the patient.

In another aspect, the invention features a kit that includes: (i) a composition that includes an antihistamine and ibudilast; and (ii) instructions for administering the composition to a patient diagnosed with or at risk of developing an immunoinflammatory disorder.

In a related aspect, the invention features a kit that includes: (i) an antihistamine; (ii) ibudilast; and (iii) instructions for administering the antihistamine and the ibudilast to a patient diagnosed with or at risk of developing an immunoinflammatory disorder.

In another aspect, the invention features a pharmaceutical composition that includes an antihistamine and rolipram. The composition may be formulated for topical administration, or for systemic administration.

In another aspect, the invention features a method of decreasing proinflammatory cytokine secretion or production in a patient by administering to the patient an antihistamine and rolipram simultaneously or within 14 days of each other in amounts sufficient to decrease proinflammatory cytokine secretion or production in the patient.

In a related aspect, the invention features a method for treating a patient diagnosed with or at risk of developing an immunoinflammatory disorder by administering to the patient an antihistamine and rolipram simultaneously or within 14 days of each other in amounts sufficient to treat the patient.

In another aspect, the invention features a kit that includes: (i) a composition that includes an antihistamine and rolipram; and (ii) instructions for administering the composition to a patient diagnosed with or at risk of developing an immunoinflammatory disorder.

In a related aspect, the invention features a kit that includes: (i) an antihistamine; (ii) rolipram; and (iii) instructions for administering the antihistamine and the rolipram to a patient diagnosed with or at risk of developing an immunoinflammatory disorder.

In another aspect, the invention features a pharmaceutical composition that includes an antihistamine and a tetra-substituted pyrimidopyrimidine. A particularly desirable tetra-substituted pyrimidopyrimidine is dipyridamole. The composition may be formulated for topical administration, or for systemic administration.

In another aspect, the invention features a method of decreasing proinflammatory cytokine secretion or production in a patient by administering to the patient an antihistamine and a tetra-substituted pyrimidopyrimidine (e.g., dipyridamole) simultaneously or within 14 days of each other in amounts sufficient to decrease proinflammatory cytokine secretion or production in the patient.

In a related aspect, the invention features a method for treating a patient diagnosed with or at risk of developing an immunoinflammatory disorder by administering to the patient an antihistamine and a tetra-substituted pyrimidopyrimidine (e.g., dipyridamole) simultaneously or within 14 days of each other in amounts sufficient to treat the patient.

In another aspect, the invention features a kit that includes: (i) a composition that includes an antihistamine and a tetra-substituted pyrimidopyrimidine; and (ii) instructions for administering the composition to a patient diagnosed with or at risk of developing an immunoinflammatory disorder.

In a related aspect, the invention features a kit that includes: (i) an antihistamine; (ii) a tetra-substituted pyrimidopyrimidine; and (iii) instructions for administering the antihistamine and the tetra-substituted pyrimidopyrimidine to a patient diagnosed with or at risk of developing an immunoinflammatory disorder.

In another aspect, the invention features a pharmaceutical composition that includes an antihistamine and a tricyclic or tetracyclic antidepressant. Particularly desirable tricyclic or tetracyclic antidepressants are nortryptiline, amoxapine, and desipramine. In one embodiment, the antihistamine is not doxepin, while in another embodiment, the antidepressant is not doxepin. The composition may be formulated for topical administration, or for systemic administration.

In another aspect, the invention features a method of decreasing proinflammatory cytokine secretion or production in a patient by administering to the patient an antihistamine and a tricyclic or tetracyclic antidepressant simultaneously or within 14 days of each other in amounts sufficient to decrease proinflammatory cytokine secretion or production in the patient. In one embodiment, the antihistamine is not doxepin, while in another embodiment, the antidepressant is not doxepin.

In a related aspect, the invention features a method for treating a patient diagnosed with or at risk of developing an immunoinflammatory disorder by administering to the patient an antihistamine and a tricyclic or tetracyclic antidepressant simultaneously or within 14 days of each other in amounts sufficient to treat the patient. In one embodiment, the antihistamine is not doxepin, while in another embodiment, the antidepressant is not doxepin.

In another aspect, the invention features a kit that includes: (i) a composition that includes an antihistamine and a tricyclic or tetracyclic antidepressant; and (ii) instructions for administering the composition to a patient diagnosed with or at risk of developing an immunoinflammatory disorder. In one embodiment, the antihistamine is not doxepin, while in another embodiment, the antidepressant is not doxepin.

In a related aspect, the invention features a kit that includes: (i) an antihistamine; (ii) a tricyclic or tetracyclic antidepressant; and (iii) instructions for administering the antihistamine and the tricyclic or tetracyclic antidepressant to a patient diagnosed with or at risk of developing an immunoinflammatory disorder.

In another aspect, the invention features a pharmaceutical composition that includes an antihistamine and a selective serotonin reuptake inhibitor (SSRI). Particularly desirable antihistamines are bromodiphenhydramine, clemizole, cyproheptadine, desloratadine, loratadine, thiethylperazine maleate, and promethazine, while particularly desirable SSRIs are paroxetine, fluoxetine, sertraline, and citalopram. The composition may be formulated for topical administration, or for systemic administration (e.g., oral administration).

In another aspect, the invention features a method of decreasing proinflammatory cytokine secretion or production in a patient by administering to the patient an antihistamine and an SSRI simultaneously or within 14 days of each other in amounts sufficient to decrease proinflammatory cytokine secretion or production in the patient.

In a related aspect, the invention features a method for treating a patient diagnosed with or at risk of developing an immunoinflammatory disorder by administering to the patient an antihistamine and an SSRI simultaneously or within 14 days of each other in amounts sufficient to treat the patient.

In another aspect, the invention features a kit that includes: (i) a composition that includes an antihistamine and an SSRI; and (ii) instructions for administering the composition to a patient diagnosed with or at risk of developing an immunoinflammatory disorder.

In a related aspect, the invention features a kit that includes: (i) an antihistamine; (ii) an SSRI; and (iii) instructions for administering the antihistamine and the SSRI to a patient diagnosed with or at risk of developing an immunoinflammatory disorder.

In particular embodiments of any of the method of the invention, the compounds are administered within 10 days of each other, within five days of each other, within twenty-four hours of each other, or even simultaneously. The compounds may be formulated together as a single composition, or may be formulated and administered separately. One or both.compounds may be administered in a low dosage or in a high dosage, each of which is defined herein. If desired, a composition may include one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator non-steroidal immunophilin-dependent immunosuppressant, vitamin D analog, psoralen, retinoid, or 5-amino salicylic acid). The composition may be formulated, for example, for topical administration or systemic administration. Combination therapies of the invention are especially useful for the treatment of immunoinflammatory disorders in combination with other anti-cytokine agents or agents that modulate the immune response to positively effect disease, such as agents that block the action of IL-6, IL-2, IL-1, IL-12, IL-15, or TNFα (e.g., etanercept, infliximab, and adelimumab), and agents that influence cell adhesion. In this example, the combination therapy reduces the production of cytokines, etanercept or infliximab act on the remaining fraction of inflammatory cytokines, providing enhanced treatment.

In any of the methods, compositions, and kits of the invention, analogs of certain compounds may be employed in lieu of the compounds themselves. Analogs of antihistamines and other compounds are described herein. Structural analogs of a compound (e.g, ibudilast) or class of compound (e.g., antihistamines) do not need to have the same activity as the compound or class to which it is related. Thus, an SSRI analog does not necessarily inhibit serotonin reuptake.

Immunoinflammatory disorders that may be treated by this method are provided herein, and include rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

By “tricyclic or tetracyclic antidepressant” is meant a compound having one the formulas (I), (II), (III), or (IV): 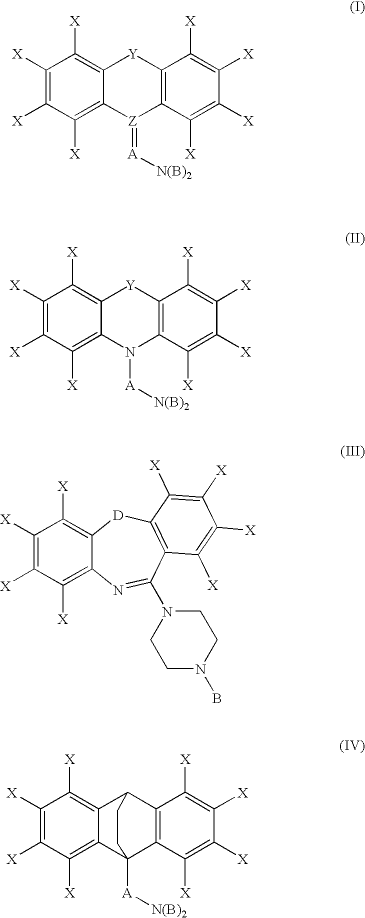
wherein each X is, independently, H, Cl, F, Br, I, CH₃, CF₃, OH, OCH₃, CH₂CH₃, or OCH₂CH₃;Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, S(O)₀₋₂. In preferred embodiments, each X is, independently, H, Cl, or F; Y is (CH₂)₂, Z is C; A is (CH₂)₃; and each B is, independently, H, Cl, or F.

By “antihistamine” is meant a compound that blocks the action of histamine. Classes of antihistamines include but are not limited to, ethanolamines, ethylenediamine, phenothiazine, alkylamines, piperazines, and piperidines

By “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are fluoxetine, fluvoxamine, paroxetine, sertraline, citalopram, and venlafaxine.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which binds to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By a “low dosage” is meant at least 5% less (e.g., at least 10%, 20%, 50%, 100%, 200%, or even 300%) than the lowest standard recommended dosage of a particular compound for treatment of any human disease or condition.

By a “high dosage” is meant at least 5% (e.g., at least 10%, 20%, 50%, 100%, 200%, or even 300%) more than the highest standard recommended dosage of a particular compound for treatment of any human disease or condition

By a “moderate dosage” is meant the dosage between the low dosage and the high dosage.

By “treating” is meant administering or prescribing a pharmaceutical composition for the treatment or prevention of an immunoinflammatory disease.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, in a combination of the invention, required to treat or prevent an immunoinflammatory disease in a clinically relevant manner. A sufficient amount of an active compound used to practice the present invention for therapeutic treatment of conditions caused by or contributing to an immunoinflammatory disease varies depending upon the manner of administration, the age, body weight, and general health of the patient. Ultimately, the prescribers will decide the appropriate amount and dosage regimen. Additionally, an effective amount may can be that amount of compound in the combination of the invention that is safe and efficacious in the treatment of a patient having the immunoinflammatory disease over each agent alone as determined and approved by a regulatory authority (such as the U.S. Food and Drug Administration).

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scieroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis.

“Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcomeal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both a proliferative skin disease and an inflammatory dermatosis. An example of such a disease is psoriasis.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₇ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₇ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

By “C₂₋₇ alkenyl” is meant a branched or unbranched hydrocarbon group containing one or more double bonds and having. from 2 to 7 carbon atoms. A C₂₋₇ alkenyl may optionally include monocyclic or polycyclic rings, in which each ring desirably has from three to six members. The C₂₋₇ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₇ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₇ alkynyl” is meant a branched or unbranched hydrocarbon group containing one or more triple bonds and having from 2 to 7 carbon atoms. A C₂₋₇ alkynyl may optionally include monocyclic, bicyclic, or tricyclic rings, in which each ring desirably has five or six members. The C₂₋₇ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom that results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 1,4,5,6-tetrahydro pyridinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, 1,4,5,6-tetrahydro pyridinyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “acyl” is meant a chemical moiety with the formula R—C(O)—, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “amido” is meant a chemical substituent of the formula —NRR′, wherein the nitrogen atom is part of an amide bond (e.g., —C(O)—NRR′) and wherein R and R′ are each, independently, selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl, or —NRR′ forms a C₂₋₆ heterocyclyl ring, as defined above, but containing at least one nitrogen atom, such as piperidino, morpholino, and azabicyclo, among others.

By “halide” is meant bromine, chlorine, iodine, or fluorine.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

The term “pharmaceutically acceptable salt” represents those salts which are, within the scope of sound medical judgment, suitable for use in contact with the tissues of humans and lower aninials without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “fexofenadine” is meant the free base, as well as any pharmaceutically acceptable salt thereof (e.g., fexofenadine hydrochloride).

Other features and advantages of the invention will be apparent from the following detailed description, and from the claims.

### DETAILED DESCRIPTION

The invention provides therapies useful for the treatment of immunoinflammatory disorders. According to the invention, any of the foregoing conditions may be treated by administration of an effective amount of an antihistamine or analog thereof, either alone or in combination with one or more additional agents.

In one embodiment of the invention, treatment of an immunoinflammatory disorder (e.g., an inflammatory dermatosis, proliferative skin disease, organ transplant rejection, or graft versus host disease) is performed by administering an antihistamine (or analog thereof) and a corticosteroid to a patient in need of such treatment.

In another embodiment of the invention, treatment of an immunoinflammatory disorder is performed by administering an antihistamine (or analog thereof) and a tricyclic or tetracyclic antidepressant to a patient in need of such treatment.

In yet another embodiment of the invention, treatment is performed by administering an antihistamine (or analog thereof) and a selective serotonin reuptake inhibitor to a patient suffering from any of the foregoing conditions.

In still other embodiments, treatment is performed by administering to a patient in need of such treatment, in conjunction with an antihistamine or antihistamine analog, dipyridamole, ibudilast, rolipram, or an analog of any of these compounds.

Exemplary routes of administration for the various embodiments can include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration refers to all nondermal routes of administration, and specifically excludes topical and transdermal routes of administration.

Any of the foregoing therapies may be administered with conventional pharmaceuticals useful for the treatment of immunoinflammatory disorders.

Antihistamines

Antihistamines are compounds that block the action of histamine. Classes of antihistamines include:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) Piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the methods, compositions, and kits of the invention, both non-sedating and sedating antihistamines may be employed. Particularly desirable antihistamines for use in the methods, compositions, and kits of the invention are non-sedating antihistamines such as loratadine and desloratadine. Sedating antihistamines can also be used in the methods, compositions, and kits of the invention. Preferred sedating antihistamines are methods, compositions, and kits of the invention are azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the methods and compositions of the invention are acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in according to the invention. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1 111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Standard Recommended Dosages

Standard recommended dosages for several exemplary antihistamines are shown in Table 1. Other standard dosages are provided, e.g., in the Merck Manual of Diagnosis & Therapy (17th Ed. M H Beers et al., Merck & Co.) and Physicians' Desk Reference 2003 (₅₇th Ed. Medical Economics Staff et al., Medical Economics Co., 2002).

TABLE 1

| Compound | Standard Dose |
|---|---|
| Desloratadine | 5 mg/once daily |
| Thiethylperazine | 10 mg/1-3 times daily |
| Bromodiphenhydramine | 12.5-25 mg/every 4-6 hours |
| Promethazine | 25 mg/twice daily |
| Cyproheptadine | 12-16 mg/day |
| Loratadine | 10 mg/once daily |
| Clemizole | 100 mg given as IV or IM |
| Azatadine | 1-2 mg/twice daily |
| Cetirizine | 5-10 mg/once daily |
| Chlorpheniramine | 2 mg/every 6 hours or |
| | 4 mg/every 6 hours |
| Dimenhydramine | 50-100 mg/every 4-6 hours |
| Diphenydramine | 25 mg/every 4-6 hours or 38 mg/ |
| | every 4-6 hours* |
| Doxylamine | 25 mg/once daily or 12.5 mg/ |
| | every four hours* |
| Fexofenadine | 60 mg/twice daily or 180 mg/ |
| | once daily |
| Meclizine | 25-100 mg/day |
| Pyrilamine | 30 mg/every 6 hours |
| Tripelennamine | 25-50 mg/every 4 to 6 hours |
| | or 100 mg/twice daily |
| | (extended release) |

Loratadine

Loratadine (CLARITIN) is a tricyclic piperidine that acts as a selective peripheral histamine H1-receptor antagonist. We report herein that loratadine and structural and functional analogs thereof, such as piperidines, tricyclic piperidines, histamine H1-receptor antagonists, are useful in the anti-immunoinflammatory combination of the invention for the treatment of immunoinflammatory disorders, transplanted organ rejection, and graft versus host disease.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Loratadine Standard Recommended Dosages Loratadine oral forrniulations include tablets, redi-tabs, and syrup. Loratadine tablets contain 10 mg micronized loratadine. Loratadine syrup contains 1 mg/ml micronized loratadine, and reditabs (rapidly-disintegrating tablets) contain 10 mg micronized loratadine in tablets that disintegrate quickly in the mouth. While suggested dosages will vary with a patient's condition, standard recommended dosages are provided below. Loratadine is typically administered once daily in a 10 mg dose, although other daily dosages useful in the anti-immunoinflammatory combination of the invention include 0.01-0.05 mg, 0.05-1 mg, 1-3 mg, 3-5 mg, 5-10 mg, 10-15 mg, 15-20 mg, 20-30 mg, and 30-40 mg.

Loratadine is rapidly absorbed following oral administration. It is metabolized in the liver to descarboethoxyloratadine by cytochrome P450 3A4 and cytochrome P450 2D6. Loratadine metabolites are also useful in the anti-immunoinflammatory combination of the invention.

Corticosteroids

If desired, one or more corticosteroid may be administered in a method of the invention or may be formulated with an antihistamine or analog thereof in a composition of the invention. Our data show that various antihistamines in combination with various corticosteroids are more effective in suppressing TNFα in vitro than either agent alone. Accordingly, this combination may be more effective in treating immunoinflammatory diseases, particularly those mediated by TNFα levels, than either the antihistamine or corticosteroid alone. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone.; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for various steroid/disease combinations are provided in Table 2, below.

TABLE 2
Standard Recommended Corticosteroid Dosages

| Indication | Route | Drug | Dose | Schedule |
|---|---|---|---|---|
| Psoriasis | oral | prednisolone | 7.5-60 mg | per day or divided b.i.d. |
| | oral | prednisone | 7.5-60 mg | per day or divided b.i.d. |
| Asthma | inhaled | beclomethasone dipropionate | 42 μg/puff) | 4-8 puffs b.i.d. |
| | inhaled | budesonide | (200 μg/inhalation) | 1-2 inhalations b.i.d. |
| | inhaled | flunisolide | (250 μg/puff) | 2-4 puffs b.i.d. |
| | inhaled | fluticasone propionate | (44, 110 or 220 μg/puff) | 2-4 puffs b.i.d. |
| | inhaled | triamcinolone acetonide | (100 μg/puff) | 2-4 puffs b.i.d. |
| COPD | oral | prednisone | 30-40 mg | per day |
| Crohn's disease | oral | budesonide | 9 mg | per day |
| Ulcerative colitis | oral | prednisone | 40-60 mg | per day |
| | oral | hydrocortisone | 300 mg (IV) | per day |
| | oral | methylprednisolone | 40-60 mg | per day |
| Rheumatoid arthritis | oral | prednisone | 10 mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (₅₇th Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Steroid Receptor Modulators

Steroid receptor modulators (e.g., antagonists and agonists) may be used as a substitute for or in addition to a corticosteroid in the methods, compositions, and kits of the invention. Thus, in one embodiment, the invention features the combination of a tricyclic compound and a glucocorticoid receptor modulator or other steroid receptor modulator, and methods of treating immunoinflammatory disorders therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Ibudilast

We have discovered that antihistamines in combination with ibudilast are more effective in suppressing TNFα in vitro than the agents alone. Accordingly, the combination of antihistamine or antihistamine analogs may be more effective in treating immunoinflammatory diseases, particulary those mediated by TNFα, than either agent alone.

An antihistamine or an antihistamine analog may be administered or formulated with ibudilast or an ibudilast analog, defined by formula (V).

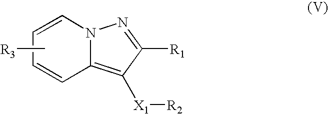

In formula (V) R₁ and R₂ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₃ is selected from H, halide, alkoxy, and C₁₋₄ alkyl; X₁ is selected from C═O, C═N—NH—R₄, C═C(R₅)—C(O)—R₆, C═CH═CH—C(O)—R₆, and C(OH)—R₇; R₄ is selected from H and acyl; R₅ is selected from H, halide, and C₁₋₄ alkyl; R₆ is selected from OH, alkoxy and amido; and R₇ is selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. Compounds of formula (V) include, the compounds described in U.S. Pat. Nos. 3,850,941; 4,097,483; 4,578,392; 4,925,849; 4,994,453; and 5,296,490. Commercially available compounds of formula (V) include ibudilast and KC-764.

The standard recommended dosage for the treatment of bronchial asthma is typically 10 mg of ibudilast twice daily, while in the case of cerebrovascular disorders, the standard recoomended dosage is 10 mg of ibudilast three times daily.

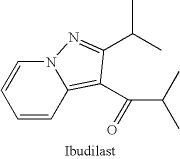

KC-764 (CAS 94457-09-7) is reported to be a platelet aggregation inhibitor.

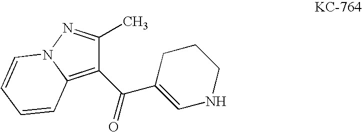

KC-764 and other compound of formula (V) can be prepared using the synthetic methods described in U.S. Pat. Nos. 3,850,941; 4,097,483; 4,578,392; 4,925,849; 4,994,453; and 5,296,490.

Rolipram

We have discovered that antihistamines in combination with rolipram are more effective in suppressing TNFα in vitro than the agents alone. Accordingly, the combination of antihistamine or antihistamine analog in combination with rolipram or rolipram analogs may be more effective in treating immunoinflammatory diseases, particulary those mediated by TNFα, than either agent alone.

In one embodiment of the invention, an antihistamine or analog thereof is administered or formulated with rolipram (4-[3-(cyclopentyloxy)-4-methoxyphenyl]-2-pyrrolidone) or an analog of rolipram. Rolipram analogs are described by formula (I) of U.S. Pat. No. 4,193,926, hereby incorporated by reference.

Tetra-substituted Pyrimidopyrimidines

We have discovered that antihistamines in combination with dipyridamole are more effective in suppressing TNFα in vitro than the agents alone. Accordingly, the combination of antihistamine or antihistamine analog in combination with a tetra-substituted pyrimidopyrimidines may be more effective in treating immunoinflammatory diseases, particulary those mediated by TNFα, than either agent alone.

In one embodiment of the invention, an antihistamine or analog thereof is administered or formulated with a tetra-substituted pyrimidopyrimidine having the formula (VI): 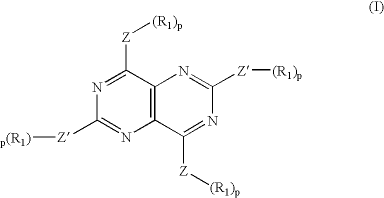
wherein each Z and each Z′ is, independently, N, O, C,

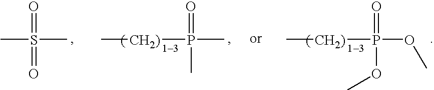

When Z or Z′ is O or 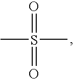
then p=1, when Z or Z′ is N, 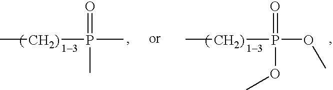
then p=2, and when Z or Z′ is C, then p=3. In formula (I), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, preferably as defined in formula (Y), below. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Exemplary tetra-substituted pyrimidopyrimidines that are useful in the methods and compositions of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. No. 3,031,450, hereby incorporated by reference.

The standard recommended dosage for dipyridamole is 300-400 mg/day.

Tricyclic and Tetracyclic Antidepressants

We have discovered that antihistamines in combination with various tricyclic and tetracyclic antidepressants are more effective in suppressing TNFα in vitro than the agents alone. Accordingly, the combination of antihistamine or antihistamine analog in combination with tricyclic and tetracyclic antidepressants and their analogs may be more effective in treating immunoinflammatory diseases, particulary those mediated by TNFα, than either agent alone.

In one embodiment of the invention, an antihistamine or analog thereof is administered or formulated with a tricyclic or tetracyclic antidepressant, or an analog thereof. By “tricyclic or tetracyclic antidepressant analog” is meant a compound having one the formulas (I), (II), (III), or (IV): 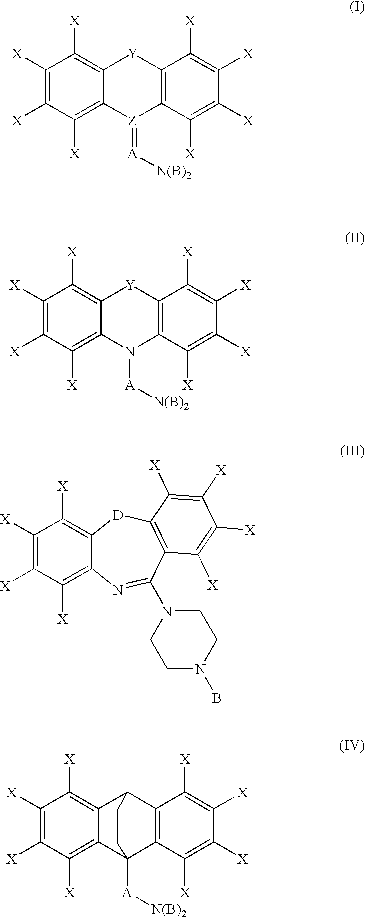
or a pharmaceutically acceptable salt, ester, amide, or derivative thereof, wherein each X is, independently, H, Cl, F, Br, I, CH₃, CF₃, OH, OCH₃, CH₂CH₃, or OCH₂CH₃;Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, S(O)₀₋₂.

In preferred embodiments, each X is, independently, H, Cl, or F; Y is (CH₂)₂, Z is C; A is (CH₂)₃; and each B is, independently, H, Cl, or F.

Tricyclic or tetracyclic antidepressants, as well as analogs thereof that are suitable for use in the methods and compositions of the invention, include 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; 8-chloro-2-methoxy-11-(4-methyl-1-piperazinyl)-5H-dibenzo(b,e)(1,4)diazepine; (Z)-2-butenedioate; 7-hydroxyamoxapine; 8-hydroxyamoxapine; 8-hydroxyloxapine; Adinazolam; Amineptine; amitriptyline; amitriptylinoxide; amoxapine; butriptyline; clomipramine; clothiapine; clozapine; demexiptiline; desipramine; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-1-(4-methyl-1-piperazinyl)-dibenz(b,f)(1,4)oxazepine monohydrochloride; 11-(4-methyl-1-piperazinyl)-dibenzo(b,f)(1,4)thiazepine; dibenzepin; dimetacrine; dothiepin; doxepin; fluacizine; fluperlapine; imipramine; imipramine N-oxide; iprindole lofepramine; loxapine; loxapine hydrochloride; loxapine succinate; maprotiline; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; nortriptyline; noxiptilin; octriptyline; opipramol; oxaprotiline; perlapine; pizotyline; propizepine; protriptyline; quetiapine; quinupramine; tianeptine; tomoxetine; and trimipramine. Others are described in U.S. Pat. Nos. 4,933,438 and 4,931,435.

Standard recommended dosages for several tricyclic antidepressants are provided in Table 3, below. Other standard dosages are provided, e.g., in the Merck Manual of Diagnosis & Therapy (17th Ed. M H Beers et al., Merck & Co.) and Physicians' Desk Reference 2003 (₅₇th Ed. Medical Economics Staff et al., Medical Economics Co., 2002).

TABLE 3

| Compound | Standard Dose |
|---|---|
| Amoxapine | 200-300 mg/day |
| Nortriptyline | 75-150 mg/day |
| Desipramine | 100-200 mg/day |

Selective Serotonin Reuptake Inhibitors

We have discovered that antihistamines in combination with various SSRI's are more effective in suppressing TNFα in vitro than the agents alone. Accordingly, the combination of antihistamine or antihistamine analog in combination with SSRIs or their analogs may be more effective in treating immunoinflammatory diseases, particulary those mediated by TNFα, than either agent alone.

In one embodiment of the invention, an antihistamine or analog thereof is administered or formulated with an SSRI or an analog thereof. Suitable SSRIs and SSRI analogs include 1,2,3,4-tetrahydro-N-methyl-4-phenyl-1-naphthylamine hydrochloride, 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; citalopram; xitalopram hydrobromide; CP 53261; didesmethylcitalopram; escitalopram; escitalopram oxalate; femoxetine, fluoxetine; fluoxetine hydrochloride; fluvoxamine; fluvoxamine maleate; indalpine, indeloxazine hydrochloride, Lu 19005; milnacipran; monodesmethylcitalopram; N-(3-fluoropropyl)paroxetine; norfluoxetine; O-desmethylvenlafaxine; paroxetine; paroxetine hydrochloride; paroxetine maleate; sertraline; sertraline hydrochloride; tametraline hydrochloride; venlafaxine; venlafaxine hydrochloride; WY 45,818; WY 45,881, and zimeldine. Other SSRI or SSRI analogs useful in the methods and compositions of the invention are described in U.S. Pat. Nos. 3,912,743; 4,007,196; 4,136,193; 4,314,081; and 4,536,518, each hereby incorporated by reference.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

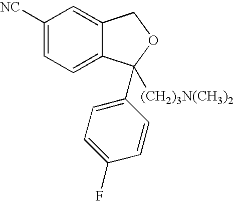

Structural analogs of citalopram are those having the formula: 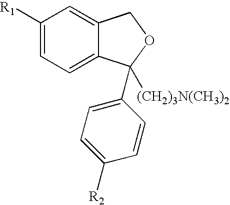
as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:.

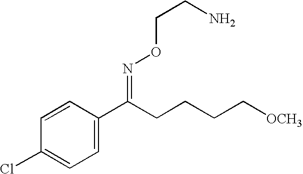

Structural analogs of clovoxamine are those having the formula: 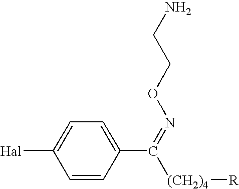
as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

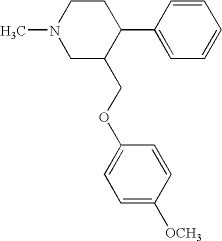

Structural analogs of femoxetine are those having the formula: 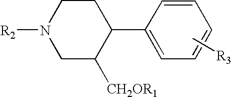
wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride ((±)-N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine. Fluoxetine hydrochloride may also be administered as an oral solution equivalent to 20 mg/5 mL of fluoxetine. A delayed release formulation contains enteric-coated pellets of fluoxetine hydrochloride equivalent to 90 mg of fluoxetine. A dose of 20 mg/day, administered in the morning, is typically recommended as the initial dose. A dose increase may be considered after several weeks if no clinical improvement is observed. Doses above 20 mg/day may be administered on a once a day (morning) or twice a day schedule (e.g., morning and noon) and should not exceed a maximum dose of 80 mg/day.

Fluoxetine has the following structure: 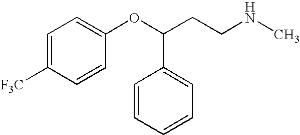
Structural analogs of fluoxetine are those compounds having the formula: 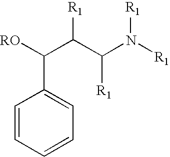
as well as pharmaceutically acceptable salts thereof, wherein each R₁ is independently hydrogen or methyl; R is naphthyl or 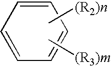
wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine B-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl) valerophenone (E)-O-(2-aminoethyl)oxime maleate. Fluvoxamine maleate is supplied as 50 mg and 100 mg tablets. Treatment is typically initiated at 50 mg given once daily at bedtime, and then increased to 100 mg daily at bedtime after a few days, as tolerated. The effective daily dose usually lies between 100 and 200 mg, but may be administered up to a maximum of 300 mg.

Fluvoxamine has the following structure:

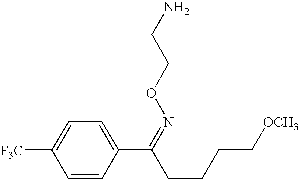

Structural analogs of fluvoxamine are those having the formula: 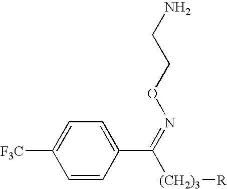
as well as pharmaceutically acceptable salts thereof, wherein R is cyano, cyanomethyl, methoxymethyl, or ethoxymethyl.

Indalpine

Indalpine has the following structure: 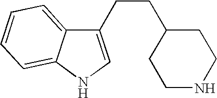
Structural analogs of indalpine are those having the formula: 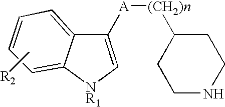
or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxezine has the following structure:

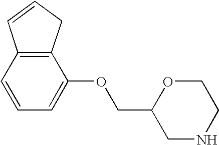

Structural analogs of indeloxazine are those having the formula: 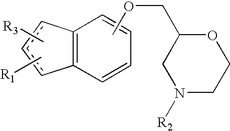
and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipran (IXEL™, Cypress Bioscience Inc.) has the chemical formula (Z)-1-diethylaminocarbonyl-2-aminoethyl-1-phenyl-cyclopropane)hydrochlorate, and is provided in 25 mg and 50 mg tablets for oral administration. It is typically administered in dosages of 25 mg once a day, 25 mg twice a day, or 50 mg twice a day for the treatment of severe depression.

Milnacipram has the following structure:

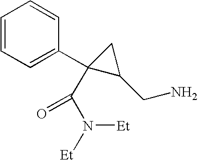

Structural analogs of milnacipram are those having the formula: 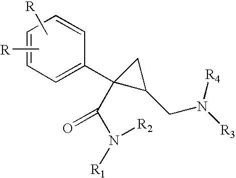
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride ((−)-trans-4 R-(4′-fluorophenyl)-3 S-[(3′,4′-methylenedioxyphenoxy) methyl] piperidine hydrochloride hemihydrate) is provided as PAXIL™. Controlled-release tablets contain paroxetine hydrochloride equivalent to paroxetine in 12.5 mg, 25 mg, or 37.5 mg dosages. One layer of the tablet consists of a degradable barrier layer and the other contains the active material in a hydrophilic matrix. TM The recommended initial dose of PAXIL™ is 25 mg/day. Some patients not responding to a 25 mg dose may benefit from dose increases, in 12.5 mg/day increments, up to a maximum of 62.5 mg/day. Dose changes typically occur at intervals of at least one week.

Paroxetine has the following structure:

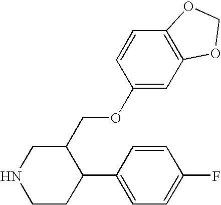

Structural analogs of paroxetine are those having the formula: 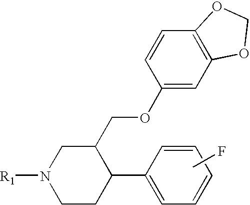
and pharmaceutically acceptable salts thereof, wherein R₁ represents hydrogen or a C₁₋₄ alkyl group, and the fluorine atom may be in any of the available positions.

Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in an anti-inflammatory combination of the invention. The metabolism of sertraline includes, for example, oxidative N-demethylation to yield N-desmethylsertraline (nor-sertraline). ZOLOFT is typically administered at a dose of 50 mg once daily.

Sertraline has the following structure:

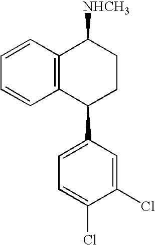

Structural analogs of sertraline are those having the formula: 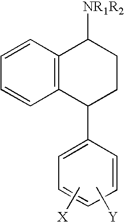
wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N,N-dimethyl-(alpha)-(2-methylpropyl)-, hydrochloride, monohydrate. Each MERIDIA™ capsule contains 5 mg, 10 mg, or 15 mg of sibutramine hydrochloride monohydrate. The recommended starting dose of MERIDIA™ is 10 mg administered once daily with or without food. If there is inadequate weight loss, the dose may be titrated after four weeks to a total of 15 mg once daily. The 5 mg dose is typically reserved for patients who do not tolerate the 10 mg dose.

Zimeldine

Zimeldine has the following structure: 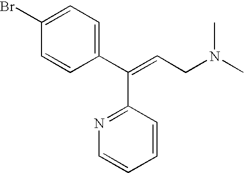
Structural analogs of zimeldine are those compounds having the formula: 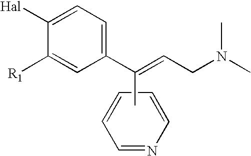
and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be employed in any of the methods, compositions, and kits of the invention.

Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

Analogs

Functional analogs of SSRIs can also be used in the methods, compositions, and kits of the invention. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno [2,3-d] pyrimidine.

Venlafaxine

Venlafaxine hydrochloride (EFFEXOR™) is an antidepressant for oral administration. It is designated (R/S)-1-[2-(dimethylamino)-1-(4-methoxyphenyl)ethyl] cyclohexanol hydrochloride or (±)-1-[(alpha)-[(dimethyl-amino)methyl]-p-methoxybenzyl] cyclohexanol hydrochloride. Compressed tablets contain venlafaxine hydrochloride equivalent to 25 mg, 37.5 mg, 50 mg, 75 mg, or 100 mg venlafaxine. The recommended starting dose for venlafaxine is 75 mg/day, administered in two or three divided doses, taken with food. Depending on tolerability and the need for further clinical effect, the dose may be increased to 150 mg/day. If desirable, the dose can be further increased up to 225 mg/day. When increasing the dose, increments of up to 75 mg/day are typically made at intervals of no less than four days.

Venlafaxine has the following structure:

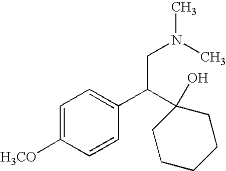

Structural analogs of venlafaxine are those compounds having the formula: 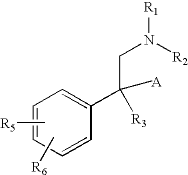
as well as pharmaceutically acceptable salts thereof, wherein A is a moiety of the formula: 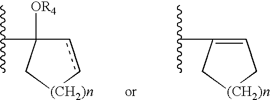
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

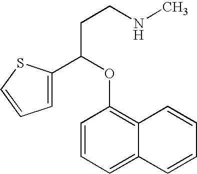

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno [2,3-d] pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

SSRI Standard Recommended Dosages

Standard recommended dosages for exemplary SSRIs are provided in Table 4, below. Other standard dosages are provided, e.g., in the Merck Manual of Diagnosis & Therapy (17th Ed. M H Beers et al., Merck & Co.) and Physicians' Desk Reference 2003 (57th Ed. Medical Economics Staff et al., Medical Economics Co., 2002).

TABLE 4

| Compound | Standard Dose |
|---|---|
| Fluoxetine | 20-80 mg/day |
| Sertraline | 50-200 mg/day |
| Paroxetine | 20-50 mg/day |
| Fluvoxamine | 50-300 mg/day |
| Citalopram | 10-80 mg qid |
| Escitalopram | 10 mg qid |

Other Compounds

The suppression of cytokine secretion or production and the treatment of the immuninflammatory disorder may be achieved by administering, in addition to one or more of the compounds described above, one or more compounds selected from methotrexate, hydroxychloroquine, sulfasalazine, tacrolimus, sirolimus, mycophenolate mofetil, and/or methyl prednisolone. A hyperproliferative skin disease (e.g., psoriasis) is conventionally treated with topical agents including coal tar, calcipotriene, and/or corticosteroids.

Nonsteroidal Immunophilin-Dependent Immunosuppressants

We have discovered that antihistamines in combination with various nonsteroidal immunophilin-dependent immunosupressants (NsIDIs) are more effective in suppressing TNFα in vitro than the agents alone. Accordingly, the combination of antihistamine or antihistamine analog in combination with immunophilin dependant immunosupressants and their analogs may be more effective in treating immunoinflammatory diseases, particulary those mediated by TNFα, than either agent alone.

In one embodiment, the NsIDI is cyclosporine, and is administered in an amount between 0.05 and 50 milligrams per kilogram per day (e.g., orally in an amount between 0.1 and 12 milligrams per kilogram per day). In another embodiment, the NsIDI is tacrolimus and is administered in an amount between 0.0001-20 milligrams per kilogram per day (e.g., orally in an amount between 0.01-0.2 milligrams per kilogram per day). In another embodiment, the NsIDI is rapamycin and is administered in an amount between 0.1-502 milligrams per day (e.g., at a single loading dose of 6 mg/day, followed by a 2 mg/day maintenance dose). In another embodiment, the NsIDI is everolimus, administered at a dosage of 0.75-8 mg/day. In still other embodiments, the NsIDI is pimecrolimus, administered in an amount between 0.1 and 200 milligrams per day (e.g., as a 1% cream/twice a day to treat atopic dermatitis or 60 mg a day for the treatment of psoriasis), or the NsIDI is a calcineurin-binding peptide administered in an amount and frequency sufficient to treat the patient. Two or more NsIDIs can be administered contemporaneously.

In healthy individuals the immune system uses cellular effectors, such as B and T cells, to target infectious microbes and abnormal cell types while leaving normal cells intact. In individuals with an autoimmune disorder or a transplanted organ, activated T cells damage healthy tissues. Calcineurin inhibitors (e.g., cyclosporines, tacrolimus, pimecrolimus) and rapamycin target many types of immunoregulatory cells, including T cells and suppress the immune response in organ transplantation and autoimmune disorders.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more.fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)—8-Cs, and D-Ser-8-Cs, which are described in Cruz et al. (Antimicrob. Agents Chemother. 44:143-149, 2000). Cyclosporines are highly hydrophobic and readily precipitate in the presence of water (e.g. on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To overcome the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL).

Typically, patient dosage of an oral cyclosporine varies according to the patient's condition, but some standard recommended dosages are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. A suggested dosing schedule is shown in Table 5. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids.

TABLE 5

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 | 0.5-4 mg/kg/day | 6-8 mg/kg/day | 6-8 mg/kg/day | ˜7-12 mg/kg/day | 2.2-6.0 mg/kg/day |
| (NEORAL) | | mg/kg/day | | (oral- | (oral) | | |
| | | | | fistulizing) | | | |
| Tacrolimus | 0.03-0.1% | 0.05-1.15 | 1-3 mg/day | 0.1-0.2 mg/kg/day | 0.1-0.2 mg/kg/day | 0.1-0.2 mg/kg/day | N/A |
| | cream/ | mg/kg/day | (oral) | (oral) | (oral) | (oral) | |
| | twice day | (oral) | | | | | |
| | (30 and | | | | | | |
| | 60 gram | | | | | | |
| | tubes) | | | | | | |
| Pimecrolimus | 1% | 40-60 | 40-60 mg/day | 80-160 mg/day | 160-240 mg/day | 40-120 mg/day | 40-120 mg/day |
| | cream/ | mg/day | (oral) | (oral) | (oral) | (oral) | (oral) |
| | twice | (oral) | | | | | |
| | day | | | | | | |
| | (15, 30, | | | | | | |
| | 100 gram | | | | | | |
| | tubes) | | | | | | |

Table Legend
CsA = cyclosporine A
RA = rheumatoid arthritis
UC = ulcerative colitis
SLE = systemic lupus erythamatosus

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages are provided below. Typically patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis. Pimecrolimus is currently available as a 1% cream. Suggested dosing schedule for pimecrolimus is shown at Table 5. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic 5 lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Patent No. 5,504,091); fluorinated esters (U.S. Patent No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Rapamycin is currently available for oral administration in liquid and tablet formulations. RAPAMUNE liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 0.5-2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1 mg/m²/day maintenance dose is used.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcineurin inhibitors, these agents are useful in the methods of the invention.

Therapy

The invention features methods for suppressing secretion of proinflammatory cytokines as a means for treating an immunoinflammatory disorder, proliferative skin disease, organ transplant rejection, or graft versus host disease.

In particular embodiments of any of the methods of the invention, the compounds are administered within 10 days of each other, within five days of each other, within twenty-four hours of each other, or simultaneously. The compounds may be formulated together as a single composition, or may be formulated and administered separately. One or both compounds may be administered in a low dosage or in a high dosage, each of which is defined herein. It may be desirable to administer to the patient other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the invention are especially useful for the treatment of immunoinflammatory disorders in combination with other agents—either biologics or small molecules—that modulate the immune response to positively affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNFα. Agents that inhibit TNFα include etanercept, adelimumab, infliximab, and CDP-870. In this example (that of agents blocking the effect of TNFα), the combination therapy reduces the production of cytokines, etanercept or infliximab act on the remaining fraction of inflammatory cytokines, providing enhanced treatment. Small molecule immunodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

Routes of administration for the various embodiments include, but are not limited to, topical, transdermal, nasal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Desirably, the methods, compositions, and kits of the invention are more effective than other methods, compositions, and kits. By “more effective” is meant that a method, composition, or kit exhibits greater efficacy, is less toxic, safer, more convenient, better tolerated, or less expensive, or provides more treatment satisfaction than another method, composition, or kit with which it is being compared.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to a corticosteroid in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the invention features the combination of a tricyclic compound and a bronchodilator, and methods of treating COPD therewith.

Psoriasis

The methods, compositions, and kits of the invention may be used for the treatment of psoriasis. If desired, one or more antipsoriatic agents typically used to treat psoriasis may be used as a substitute for or in addition to a corticosteroid in the methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of a tricyclic compound and an antipsoriatic agent, and methods of treating psoriasis therewith.

Inflammatory Bowel Disease

The methods, compositions, and kits of the invention may be used for the treatment of inflammatory bowel disease. If desired, one or more agents typically used to treat inflammatory bowel disease may be used as a substitute for or in addition to a corticosteroid in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of a tricyclic compound and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used as a substitute for or in addition to a corticosteroid in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, mel6xicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of a tricyclic compound with any of the foregoing agents, and methods of treating rheumatoid arthritis therewith.

Asthma

The methods, compositions, and kits of the invention may be used for the treatment of asthma. If desired, one or more agents typically used to treat asthma may be used as a substitute for or in addition to a corticosteroid in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of a tricyclic compound and any of the foregoing agents, and methods of treating asthma therewith.

Formulation of Pharmaceutical Compositions

Suitable modes of administration include topical or transdermal, oral, rectal, intravenous, intramuscular, subcutaneous, inhalation, vaginal, intraperitoneal (IP), intraarticular, and ophthalmic.

The invention can also be provided as components of a pharmaceutical pack. The two drugs can be formulated together or separately and in individual dosage amounts.

Administration of each component of the combination may be by any suitable means that is effective for the treatment of an immunoinflammatory disorder, proliferative skin disease, organ transplant rejection, or graft versus host disease. Compounds are admixed with a suitable carrier substance, and are generally present in an amount of 0.1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds. J. Swarbrick and J. C. Boylan, 1988-2002, Marcel Dekker, New York).

Pharmaceutical compositions according to the invention may be formulated to release the active compound substantially immediately upon administration or at any predetermined time period after administration, using controlled release formulations.

Administration of compounds in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastrointestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Topical Formulations

For compositions adapted for topical use, a topical vehicle containing from between 0.1% to 25% (w/w) or more of antihistamine or analog and/or additional agent, preferably from between 0.1% to 10% (w/w), more preferably from between 0.05% to 4% (w/w) active agent. The cream can be applied one to four times daily, or as needed.

For example, for prednisolone adapted for topical administration, a topical vehicle will contain from between 0.01% to 5% (w/w), preferably from between 0.01% to 2% (w/w), more preferably from between 0.01% to 1% (w/w) prednisolone.

Performing the methods described herein, the topical vehicle containing a compound of anthistamine or antihistamine analog, and/or the additional agent is preferably applied to the site of discomfort on the subject. For example, a cream may be applied to the hands of a subject suffering from arthritic fingers, while topical eye drops may be applied to an eye of a subject to treat uveitis.

Dosages

Given the enhanced potency of the combinations of the invention, it is understood that a low dosage (as defined herein) of the antihistamine and/or the additional agents can be used. These dosages will vary depending on the health and condition of the patient. Thus, a moderate dosage or even a high dosage of one or both agents can be used. Administration of each drug in the combination can, independently, be one to four times daily for one day to one year, and may even be for the life of the patient. Chronic, long-term administration will be indicated in many cases.

Additional Applications

The compounds of the invention are also useful as screening tools. Single agents and combinations of the invention can be employed in antiproliferative or mechanistic assays to determine whether other combinations, or single agents are as effective as the combination in inhibiting the proliferation of proinflammatory cytokines using assays generally known in the art, e.g., TNFα, IL-2, etc., specific, non-limiting examples of which are described in the Examples section. For example, candidate compounds are combined with a compound from with either the antihistamine (or antihistamine analog) or the additional agents described herein, applied to stimulated PBMCs, and at after a suitable time, the cells are examined for anitproliferative activity, TNFα or other assays for proinflammatory cytokine secretion. The relative effects of the combinations versus each other, and versus the single agents are compared, and effective compounds and combinations are identified. The screening method can be used for comparing the activity of novel single agents or new combinations of agents (novel or known) for relative activity in the assays.

The combinations of the invention are also useful tools in elucidating mechanistic information about the biological pathways involved in inflammation or novel targets. Such information can lead to the development of new combinations or single agents (mechanistic and/or structural analogs of either the antihistamine or companion compound) for inhibiting proinflammatory cytokine secretion. Methods known in the art to determine biological pathways can be used to determine the pathway, or network of pathways affected by contacting cells stimulated to produce proinflammatory cytokines with the compounds of the invention. Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e,.g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

### EXAMPLE

The following example is to illustrate the invention, and is not meant to limit the invention in any way.

Methods

TNFα Secretion Assay

The effects of test compound combinations on TNFα secretion were assayed in white blood cells from human buffy coat stimulated with phorbol 12-myristate 13-acetate and ionomycin as follows. Human white blood cells from buffy coat were diluted 1:50 in media (RPMI; Gibco BRL, #11875-085), 10% fetal bovine serum (Gibco BRL, #25140-097), 2% penicillin/streptomycin (Gibco BRL, #15140-122)) and 50 μL of the diluted white blood cells was placed in each well of the assay plate. Drugs were added to the indicated concentration. After 16-18 hours of incubation at 37° C. with 5% CO₂ in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate was washed (Tecan Powerwasher 384) with PBS containing 0.1% Tween 20 and incubated for one additional hour with biotin labeled anti-TNFα antibody (PharMingen, #554511) and HRP coupled to streptavidin (PharMingen, #13047E). The plate was then washed again with 0.1% Tween 20/PBS. An HRP-luminescent substrate was added to each well, and the light intensity of each well was measured using a plate luminometer.

IL-2 Secretion Assay

The effects of test compound combinations on IL-2 secretion were assayed in white blood cells from human buffy coat stimulated with phorbol 12-myristate 13-acetate, as follows. Human white blood cells from buffy coat were diluted 1:50 in media (RPMI; Gibco BRL, #11875-085), 10% fetal bovine serum (Gibco BRL, #25140-097), 2% penicillin/streptomycin (Gibco BRL, #15140-122)) and 50 μL of the diluted white blood cells was placed in each well of the final assay plate created in the above section. After 16-18 hours of incubation at 37° C. in a humidified incubator, the plate was centrifuged and the supernatant was transferred to a white opaque 384-well plate (NalgeNunc, MAXISORB) coated with an anti-IL-2 antibody (PharMingen, #555051). After a two-hour incubation, the plate was washed (Tecan Powerwasher 384) with PBS containing 0.1% Tween 20 and incubated for an additional one hour with a biotin labeled anti-IL-2 antibody (Endogen, M600B) and horse radish peroxidase coupled to streptavidin (PharMingen, #13047E). The plate was then washed again with 0.1% Tween 20/PBS, and an HRP-luminescent substrate was added to each well. Light intensity was then measured using a plate luminometer.

Percent Inhibition

The percent inhibition (% I) for each well was calculated using the following formula:
% I=[(avg. untreated wells−treated well)/(avg. untreated wells)]×100
The average untreated well value (avg. untreated wells) is the arithmetic mean of 40 wells from the same assay plate treated with vehicle alone. Negative inhibition values result from local variations in treated wells as compared to untreated wells.

The results of various combinations of compounds described on the reduction of TNFα secretion are shown in Tables 6-79, while the results of various combinations on the reduction of IL-2 secretion are shown in Tables 80-115. The effects of varying concentrations of single compound or when used in combination with another compound is shown in individual tables. For example, Table 6 shows the effects of varying concentrations of prednisolone and a combination of desloratadine and prednisolone. These results were compared to control wells. These wells were stimulated with phorbol 12-myristate 13-acetate and ionomycin, but did not receive desloratadine or prednisolone. The effects of the agents alone and in combination are shown as percent inhibition of TNFα secretion.

TABLE 6

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Desloratadine (μM) | 0 | −6.2 | 1.4 | 4.2 | 13 | 20 | 26 | 30 | 35 | 37 |
| | 0.25 | −1.3 | −0.14 | 4.7 | 16 | 19 | 24 | 31 | 32 | 38 |
| | 0.51 | −2.7 | 4.4 | 10 | 16 | 23 | 29 | 34 | 35 | 38 |
| | 1 | 6.6 | 0.94 | 14 | 13 | 25 | 27 | 35 | 37 | 42 |
| | 2 | 16 | 22 | 24 | 29 | 34 | 38 | 42 | 48 | 49 |
| | 4.1 | 30 | 31 | 35 | 42 | 42 | 50 | 53 | 54 | 60 |
| | 8.1 | 58 | 63 | 62 | 65 | 68 | 73 | 73 | 76 | 77 |
| | 16 | 86 | 88 | 88 | 88 | 89 | 91 | 91 | 92 | 92 |
| | 33 | 96 | 96 | 97 | 97 | 97 | 97 | 96 | 97 | 97 |

TABLE 7

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Loratadine (μM) | 0 | 7 | 14 | 8.5 | 14 | 18 | 17 | 24 | 29 | 40 |
| | 0.2 | 6 | 14 | 16 | 15 | 18 | 25 | 33 | 33 | 39 |
| | 0.41 | 3 | 13 | 11 | 15 | 16 | 19 | 24 | 29 | 40 |
| | 0.82 | 6 | 12 | 9.4 | 11 | 24 | 23 | 26 | 31 | 39 |
| | 1.6 | 17 | 12 | 16 | 16 | 15 | 27 | 34 | 35 | 41 |
| | 3.3 | 21 | 20 | 26 | 26 | 25 | 32 | 40 | 47 | 45 |
| | 6.5 | 35 | 37 | 34 | 38 | 40 | 45 | 46 | 52 | 59 |
| | 13 | 55 | 54 | 55 | 57 | 55 | 65 | 63 | 63 | 70 |
| | 26 | 77 | 78 | 77 | 80 | 76 | 81 | 81 | 81 | 86 |

TABLE 8

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Cyproheptadine (μM) | 0 | −5 | 13 | 13 | 22 | 29 | 32 | 34 | 39 | 39 |
| | 0.24 | 0 | 14 | 20 | 24 | 30 | 39 | 38 | 42 | 42 |
| | 0.48 | 3 | 13 | 17 | 23 | 29 | 34 | 40 | 38 | 40 |
| | 0.96 | 0 | 13 | 23 | 30 | 30 | 42 | 40 | 42 | 41 |
| | 1.9 | 12 | 18 | 25 | 33 | 35 | 44 | 41 | 44 | 44 |
| | 3.9 | 14 | 31 | 34 | 41 | 45 | 46 | 47 | 52 | 53 |
| | 7.7 | 32 | 42 | 39 | 50 | 57 | 57 | 61 | 62 | 60 |
| | 15 | 54 | 62 | 61 | 67 | 67 | 71 | 71 | 70 | 72 |
| | 31 | 77 | 80 | 80 | 84 | 84 | 85 | 86 | 85 | 87 |

TABLE 9

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Thiethylperazine (μM) | 0 | −1.3 | 8.3 | 11 | 12 | 26 | 34 | 36 | 39 | 39 |
| | 0.2 | −4.9 | 9.3 | 13 | 15 | 25 | 30 | 38 | 38 | 42 |
| | 0.39 | 0.79 | 10 | 16 | 17 | 25 | 31 | 38 | 37 | 37 |
| | 0.78 | 0.21 | 7 | 4.5 | 21 | 31 | 32 | 38 | 38 | 37 |
| | 1.6 | 5.3 | 13 | 9.5 | 19 | 26 | 25 | 32 | 34 | 36 |
| | 3.1 | 8.7 | 18 | 18 | 27 | 31 | 30 | 36 | 31 | 40 |
| | 6.3 | 21 | 29 | 31 | 34 | 39 | 41 | 47 | 43 | 53 |
| | 13 | 52 | 67 | 71 | 72 | 74 | 80 | 76 | 78 | 78 |
| | 25 | 93 | 94 | 91 | 89 | 90 | 95 | 91 | 88 | 94 |

TABLE 10

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Bromodiphenhydramine HCl | 0 | 7.9 | 0.3 | 6.3 | 17 | 22 | 34 | 37 | 34 | 37 |
| (μM) | 0.21 | −0.64 | 6.6 | 5.9 | 17 | 24 | 30 | 31 | 34 | 40 |
| | 0.43 | −7 | −0.23 | 2.5 | 18 | 23 | 28 | 35 | 37 | 33 |
| | 0.85 | −3.4 | 2.7 | 6.1 | 21 | 33 | 33 | 36 | 40 | 36 |
| | 1.7 | −7 | 2.7 | 8.8 | 28 | 28 | 29 | 34 | 39 | 34 |
| | 3.4 | 2.7 | 18 | 17 | 25 | 30 | 31 | 34 | 34 | 35 |
| | 6.8 | 23 | 33 | 33 | 37 | 43 | 46 | 52 | 56 | 56 |
| | 14 | 46 | 51 | 59 | 56 | 60 | 67 | 67 | 67 | 65 |
| | 27 | 66 | 66 | 74 | 77 | 79 | 83 | 80 | 83 | 80 |

TABLE 11

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Promethazine HCl (μM) | 0 | 2.3 | −3.2 | 5.3 | 15 | 22 | 33 | 33 | 32 | 39 |
| | 0.24 | −3.5 | 3.2 | 8 | 25 | 23 | 30 | 35 | 34 | 38 |
| | 0.49 | −2.2 | −4.2 | −4.3 | 22 | 19 | 29 | 31 | 36 | 38 |
| | 0.97 | 2 | 6.2 | 11 | 14 | 28 | 26 | 30 | 29 | 32 |
| | 1.9 | 6.7 | 5 | 10 | 14 | 23 | 27 | 37 | 35 | 38 |
| | 3.9 | −3.8 | 18 | 19 | 20 | 21 | 36 | 35 | 36 | 40 |
| | 7.8 | 15 | 35 | 29 | 41 | 45 | 45 | 46 | 48 | 48 |
| | 16 | 42 | 46 | 55 | 54 | 59 | 67 | 65 | 65 | 62 |
| | 31 | 67 | 78 | 72 | 78 | 80 | 82 | 84 | 85 | 82 |

TABLE 12

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Clemizole (μM) | 0 | 1 | 4 | 8.8 | 17 | 21 | 31 | 35 | 37 | 40 |
| | 0.22 | −6 | 5 | 11 | 16 | 25 | 28 | 33 | 38 | 36 |
| | 0.43 | −1 | 6 | 10 | 18 | 26 | 30 | 38 | 37 | 39 |
| | 0.86 | −3 | 4 | 11 | 21 | 28 | 32 | 36 | 36 | 36 |
| | 1.7 | 0 | 3 | 13 | 24 | 28 | 34 | 34 | 35 | 34 |
| | 3.5 | 3.2 | 6 | 17 | 22 | 28 | 36 | 35 | 33 | 38 |
| | 6.9 | 14 | 22 | 23 | 31 | 39 | 42 | 47 | 44 | 45 |
| | 14 | 30 | 32 | 33 | 46 | 46 | 54 | 56 | 57 | 54 |
| | 28 | 44 | 56 | 54 | 56 | 62 | 66 | 65 | 70 | 68 |

TABLE 13

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Amoxapine (μM) | 0 | −3.901 | 1.183 | −2.478 | −4.702 | −2.172 | −0.085 | 7.412 | 20 | 42.5 |
| | 0.2 | 4.199 | 5.515 | 7.33 | 8.65 | 10.5 | 14.77 | 15.73 | 36.9 | 57.15 |
| | 0.4 | 13.53 | 11.6 | 9.42 | 13.82 | 13.76 | 15.5 | 24.7 | 41.77 | 64.05 |
| | 0.8 | 24.58 | 25.35 | 22.48 | 24.62 | 27.1 | 29.27 | 36.77 | 50.5 | 74.22 |
| | 1.6 | 36.15 | 39.08 | 35.9 | 38.62 | 39.67 | 44.2 | 50.18 | 63.58 | 81.1 |
| | 3.2 | 52.7 | 51.37 | 53.88 | 53.12 | 54.95 | 58.7 | 65.1 | 78.55 | 88.32 |
| | 6.4 | 68.1 | 71.03 | 68.97 | 71.8 | 72.45 | 75 | 80.02 | 86.68 | 92.72 |
| | 13 | 84.97 | 86.6 | 86.35 | 86.45 | 87.55 | 88.8 | 90.17 | 92.55 | 95.78 |
| | 25 | 93.55 | 94.6 | 94.28 | 94.6 | 94.82 | 94.57 | 95.1 | 94.47 | 96.1 |

TABLE 14

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Nortriptyline HCl (μM) | 0 | −10 | −5.25 | −9.6 | −9.34 | −11.89 | −10.52 | −10.84 | 8.955 | 28.9 |
| | 0.26 | −9.834 | −0.255 | −1.055 | −10.03 | −4.788 | −9.77 | −12.06 | 8.255 | 37.25 |
| | 0.52 | −15.8 | −11.85 | −11.75 | −6.67 | −7.776 | −4.917 | −1.5 | 15.2 | 38.05 |
| | 1 | 2.735 | 2.925 | 2.72 | 6.05 | 3.015 | 10.52 | 8.445 | 24 | 44.3 |
| | 2.1 | 8.32 | 14.7 | 15.9 | 12.11 | 13.6 | 19.4 | 18.5 | 35.2 | 56.05 |
| | 4.2 | 31.8 | 33.45 | 33.2 | 27.3 | 33.85 | 38.35 | 38.9 | 51.85 | 68.3 |
| | 8.3 | 56.25 | 58.85 | 58.35 | 55.7 | 58.65 | 63.8 | 65.9 | 75.3 | 85.15 |
| | 17 | 81.7 | 83.45 | 81.75 | 83.1 | 83.4 | 84.9 | 86.35 | 89.1 | 91.7 |
| | 33 | 92.9 | 93.25 | 93.15 | 93.35 | 94.05 | 94.05 | 92.85 | 94.75 | 92.7 |

TABLE 15

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Paroxetine HCl (μM) | 0 | −1.632 | −0.937 | 4.873 | 7.158 | 1.443 | 4.874 | 7.425 | 22.43 | 42.08 |
| | 0.21 | −2.35 | 1.458 | 6.04 | 9.973 | 10.33 | 5.633 | 10.09 | 22.08 | 48.2 |
| | 0.42 | 2.99 | 2.115 | 2.633 | 1.712 | 2.245 | 0.2125 | 14.09 | 27.23 | 47.85 |
| | 0.83 | 8.769 | 6.098 | 7.953 | 5.995 | 8.823 | 14.89 | 17.41 | 32.88 | 48.85 |
| | 1.7 | 16.92 | 18.66 | 21.18 | 19.4 | 26.62 | 20.15 | 29.27 | 38.22 | 59.4 |
| | 3.3 | 29.48 | 34.7 | 36.77 | 37.62 | 37.48 | 45.85 | 47.6 | 59.43 | 74.58 |
| | 6.7 | 60.7 | 59.52 | 61.85 | 62.37 | 62.83 | 65.2 | 69.97 | 78.7 | 85.62 |
| | 13 | 80.05 | 82.95 | 84.95 | 82.15 | 82.55 | 85.18 | 86.55 | 89.07 | 91.83 |
| | 27 | 92.45 | 94.47 | 92.75 | 92.78 | 93.1 | 90.83 | 92.85 | 91.25 | 90.97 |

TABLE 16

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Amoxapine (μM) | 0 | −3.368 | 0.62 | −2.897 | −7.15 | 3.18 | 8.702 | 28.73 | 60.38 | 85.42 |
| | 0.2 | 6.175 | 3.94 | 5.515 | 10.82 | 16.75 | 26.7 | 38.95 | 65.78 | 85.60 |
| | 0.4 | 12.12 | 18.55 | 15.81 | 15.53 | 16.97 | 29.48 | 45.78 | 68.20 | 86.38 |
| | 0.8 | 24.45 | 21.95 | 24.73 | 24.07 | 29.00 | 42.6 | 49.92 | 71.92 | 88.90 |
| | 1.6 | 38.60 | 36.62 | 42.77 | 39.33 | 45.07 | 51.35 | 61.48 | 74.45 | 89.25 |
| | 3.2 | 55.42 | 53.92 | 55.47 | 55.87 | 59.13 | 64.25 | 74.30 | 84.80 | 91.65 |
| | 6.4 | 70.35 | 70.85 | 70.30 | 71.12 | 75.53 | 77.60 | 82.10 | 88.70 | 93.83 |
| | 13 | 86.65 | 88.12 | 86.62 | 86.75 | 88.12 | 89.92 | 90.78 | 93.22 | 95.35 |
| | 25 | 93.60 | 92.03 | 94.38 | 95.08 | 94.4 | 94.6 | 95.43 | 94.45 | 94.53 |

TABLE 17

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Fluoxetine HCl (μM) | 0 | −0.83 | 6.99 | −1.065 | 1.40 | −4.03 | 11.41 | 34.3 | 65.3 | 87.05 |
| | 0.23 | −0.34 | −3.88 | 0.34 | 1.34 | 3.415 | 8.742 | 29.15 | 64.65 | 88.6 |
| | 0.45 | −0.46 | −4.17 | −1.21 | −0.575 | 1.02 | 8.39 | 34.2 | 61.95 | 87.7 |
| | 0.9 | −0.103 | −1.92 | 4.745 | 3.03 | 7.625 | 19.65 | 31.15 | 64.45 | 88.2 |
| | 1.8 | 4.12 | 4.49 | 3.09 | 1.85 | 9.195 | 13.94 | 33.5 | 60.75 | 88.3 |
| | 3.6 | 1.71 | 2.92 | 6.862 | 9.465 | 5.375 | 21.4 | 34.75 | 65.75 | 88.15 |
| | 7.2 | −2.67 | 8.64 | 2.84 | 3.936 | 9.95 | 13.73 | 35.25 | 64.7 | 89.4 |
| | 14 | 4.37 | −3.70 | 0.458 | 3.479 | 4.82 | 18.10 | 37.05 | 62.85 | 88.7 |
| | 29 | 2.89 | 3.92 | 4.245 | 6.98 | 12.5 | 18.00 | 41.05 | 72.15 | 89.25 |

TABLE 18

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Nortriptyline HCl (μM) | 0 | −3.44 | 0.273 | −2.52 | 3.702 | 0.248 | 9.043 | 27.43 | 51.42 | 81.08 |
| | 0.26 | 0.362 | −0.268 | 1.46 | 3.213 | 4.86 | 17.73 | 26.48 | 56.75 | 81.72 |
| | 0.52 | −2.66 | 1.22 | 4.29 | 4.685 | 6.00 | 13.67 | 31.67 | 56.15 | 81.08 |
| | 1 | 7.10 | 7.633 | 8.24 | 7.537 | 15.35 | 24.55 | 36.42 | 59.28 | 82.78 |
| | 2.1 | 16.73 | 20.15 | 21.35 | 25.95 | 23.93 | 32.58 | 45.88 | 62.92 | 82.85 |
| | 4.2 | 34.40 | 36.50 | 36.30 | 35.85 | 42.5 | 50.75 | 57.92 | 71.42 | 86.25 |
| | 8.3 | 60.67 | 60.90 | 65.15 | 62.92 | 66.65 | 68.35 | 73.65 | 82.57 | 90.45 |
| | 17 | 83.20 | 84.50 | 84.9 | 83.88 | 85.5 | 86.9 | 87.15 | 88.83 | 91.88 |
| | 33 | 92.33 | 93.1 | 92.8 | 91.55 | 92.02 | 91.92 | 92.33 | 91.07 | 91.03 |

TABLE 19

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Paroxetine HCl (μM) | 0 | 0.855 | −6.695 | −2.296 | 1.015 | 5.86 | 12.26 | 29.85 | 57.07 | 84.2 |
| | 0.21 | 0.703 | −0.375 | 4.13 | 2.214 | 7.413 | 15.47 | 33.83 | 60.35 | 85.7 |
| | 0.42 | 1.144 | 0.704 | 4.308 | 2.272 | 1.64 | 15.75 | 34.85 | 63.88 | 85.4 |
| | 0.83 | 0.935 | 4.725 | 5.77 | 9.678 | 11.41 | 18.38 | 40.42 | 66.03 | 87.22 |
| | 1.7 | 15.7 | 20.02 | 23.1 | 23.05 | 25.42 | 33.35 | 48.12 | 67.22 | 87.72 |
| | 3.3 | 34.68 | 35.88 | 35.6 | 39.12 | 39.58 | 50.33 | 60.33 | 78.4 | 90.62 |
| | 6.7 | 60.55 | 63.97 | 63.3 | 65.15 | 67 | 69.15 | 75.9 | 86.38 | 92.62 |
| | 13 | 85 | 87.7 | 87.12 | 86.87 | 87.85 | 89.5 | 90.65 | 93.5 | 95.75 |
| | 27 | 95.88 | 95.65 | 96.2 | 96.75 | 96.65 | 96.3 | 96.58 | 96.1 | 96.03 |

TABLE 20

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Thiethylperazine Maleate | 0 | −9.3 | −4.4 | −8.4 | −3.1 | 14 | 31 | 52 | 72 | 82 |
| (μM) | 0.2 | −7.1 | −5.3 | −5.6 | 1.8 | 13 | 41 | 51 | 68 | 64 |
| | 0.39 | −11 | −7.1 | −4 | 2.2 | 16 | 36 | 51 | 69 | 86 |
| | 0.78 | −9.7 | −10 | −9.4 | 2 | 8.8 | 34 | 53 | 74 | 79 |
| | 1.6 | −13 | −9 | −5.6 | 2.2 | 17 | 39 | 52 | 74 | 89 |
| | 3.1 | 0.11 | −2.5 | 2.1 | 9.6 | 23 | 36 | 55 | 75 | 80 |
| | 6.3 | 18 | 6.7 | 18 | 24 | 37 | 47 | 67 | 80 | 78 |
| | 13 | 50 | 58 | 62 | 56 | 70 | 73 | 69 | 86 | 59 |
| | 25 | 76 | 75 | 78 | 81 | 86 | 87 | 74 | 82 | 78 |

TABLE 21

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Bromodiphenhydramine | 0 | 3.2 | 4.3 | 13 | 20 | 37 | 48 | 65 | 80 | 92 |
| HCl (μM) | 0.21 | −2.3 | 12 | 12 | 21 | 32 | 48 | 65 | 80 | 64 |
| | 0.43 | −9.2 | 9.4 | 12 | 20 | 35 | 48 | 66 | 81 | 92 |
| | 0.85 | −4.9 | 3.7 | 18 | 17 | 33 | 49 | 66 | 80 | 92 |
| | 1.7 | 1.7 | 11 | 17 | 20 | 37 | 44 | 68 | 82 | 91 |
| | 3.4 | 13 | 12 | 20 | 28 | 40 | 55 | 69 | 81 | 91 |
| | 6.8 | 24 | 28 | 34 | 38 | 50 | 63 | 74 | 84 | 93 |
| | 14 | 43 | 51 | 53 | 58 | 70 | 74 | 82 | 88 | 95 |
| | 27 | 69 | 72 | 75 | 76 | 84 | 85 | 90 | 92 | 95 |

TABLE 22

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Cyproheptadine | 0 | −4.6 | 3.5 | 9.8 | 17 | 24 | 45 | 68 | 85 | 94 |
| HCl (μM) | 0.24 | 2.6 | 2.8 | 7.4 | 22 | 33 | 50 | 67 | 83 | 66 |
| | 0.48 | −7.8 | 7.2 | 7.5 | 21 | 34 | 51 | 69 | 83 | 92 |
| | 0.96 | −1.6 | 3.9 | 11 | 23 | 34 | 53 | 69 | 84 | 94 |
| | 1.9 | 1.5 | 12 | 17 | 25 | 41 | 56 | 72 | 86 | 93 |
| | 3.9 | 13 | 20 | 29 | 34 | 43 | 62 | 76 | 86 | 94 |
| | 7.7 | 30 | 40 | 45 | 52 | 59 | 72 | 82 | 88 | 94 |
| | 15 | 59 | 67 | 65 | 71 | 74 | 82 | 88 | 92 | 94 |
| | 31 | 80 | 84 | 84 | 88 | 90 | 91 | 93 | 90 | 94 |

TABLE 23

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Thiethylperazine Maleate (μM) | 0 | 0.13 | 12 | 12 | 24 | 33 | 50 | 65 | 74 | 83 |
| | 0.2 | 3.3 | 17 | 17 | 23 | 31 | 48 | 64 | 74 | 83 |
| | 0.39 | −10 | 4.3 | 14 | 16 | 36 | 50 | 66 | 77 | 87 |
| | 0.78 | −3.6 | 6.1 | 2.2 | 21 | 36 | 48 | 67 | 75 | 86 |
| | 1.6 | −4 | 2.8 | 17 | 23 | 36 | 53 | 62 | 75 | 81 |
| | 3.1 | 11 | 16 | 21 | 29 | 42 | 53 | 67 | 80 | 84 |
| | 6.3 | 27 | 23 | 35 | 44 | 54 | 63 | 73 | 81 | 87 |
| | 13 | 56 | 64 | 63 | 68 | 75 | 79 | 79 | 90 | 89 |
| | 25 | 85 | 78 | 86 | 85 | 91 | 90 | 86 | 85 | 86 |

TABLE 24

| | | Nortryptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Bromodiphenhydramine | 0 | −7.2 | −5.3 | −8.8 | 1.7 | 15 | 30 | 59 | 80 | 92 |
| HCl (μM) | 0.21 | −13 | −14 | −1.5 | −8.3 | 8.2 | 30 | 57 | 80 | 94 |
| | 0.43 | −16 | −15 | −12 | −4.3 | 12 | 33 | 59 | 79 | 93 |
| | 0.85 | −15 | −7.9 | −10 | −0.52 | 18 | 34 | 62 | 79 | 92 |
| | 1.7 | −15 | −8.7 | −3.7 | −1.9 | 18 | 42 | 61 | 80 | 93 |
| | 3.4 | −9.9 | 3.2 | −5.9 | 8 | 24 | 49 | 69 | 82 | 94 |
| | 6.8 | 15 | 18 | 15 | 23 | 38 | 55 | 73 | 85 | 94 |
| | 14 | 49 | 48 | 50 | 58 | 65 | 73 | 81 | 89 | 81 |
| | 27 | 74 | 76 | 74 | 77 | 83 | 85 | 88 | 93 | 90 |

TABLE 25

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Cyproheptadine HCl (μM) | 0 | −6.4 | −1.5 | −4.5 | 8.2 | 21 | 35 | 62 | 81 | 95 |
| | 0.24 | −5.4 | 9.3 | 6.5 | 14 | 25 | 33 | 57 | 82 | 93 |
| | 0.48 | −7.8 | −1.5 | −0.76 | 13 | 21 | 40 | 62 | 83 | 95 |
| | 0.96 | 0.005 | 4.5 | 8.7 | 20 | 28 | 42 | 64 | 81 | 96 |
| | 1.9 | 3.9 | 12 | 13 | 15 | 28 | 49 | 67 | 84 | 96 |
| | 3.9 | 17 | 20 | 23 | 30 | 34 | 60 | 75 | 86 | 95 |
| | 7.7 | 33 | 37 | 41 | 45 | 53 | 68 | 79 | 89 | 96 |
| | 15 | 59 | 66 | 61 | 67 | 71 | 78 | 86 | 92 | 89 |
| | 31 | 82 | 82 | 84 | 86 | 87 | 90 | 93 | 95 | 97 |

TABLE 26

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Thiethylperazine Maleate | 0 | −5 | 8.3 | −6.6 | 13 | 23 | 36 | 56 | 70 | 74 |
| (μM) | 0.2 | −2.6 | 3.1 | −1.9 | 9.9 | 19 | 37 | 56 | 69 | 65 |
| | 0.39 | −18 | 0.5 | 6.2 | 14 | 24 | 32 | 54 | 71 | 77 |
| | 0.78 | −4.8 | −1.5 | 8.2 | 16 | 27 | 28 | 58 | 71 | 68 |
| | 1.6 | −7.6 | 4.5 | 8.1 | 17 | 29 | 45 | 62 | 75 | 85 |
| | 3.1 | 6.9 | 10 | 12 | 14 | 30 | 48 | 65 | 76 | 85 |
| | 6.3 | 13 | 13 | 17 | 19 | 49 | 55 | 69 | 79 | 81 |
| | 13 | 45 | 49 | 52 | 52 | 70 | 73 | 78 | 83 | 76 |
| | 25 | 69 | 69 | 69 | 64 | 82 | 72 | 79 | 77 | 84 |

TABLE 27

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Bromodiphenhydramine | 0 | −2.2 | 8.3 | 2.2 | 7.7 | 7.4 | 36 | 56 | 66 | 75 |
| HCl (μM) | 0.21 | 7 | 0.69 | 1.3 | 11 | 16 | 40 | 55 | 66 | 69 |
| | 0.43 | 2.1 | 1 | −0.63 | 1.5 | 19 | 32 | 50 | 66 | 72 |
| | 0.85 | −5.6 | −0.5 | −1.9 | 1.1 | 20 | 28 | 46 | 64 | 72 |
| | 1.7 | −14 | −4 | 0.74 | 11 | 23 | 39 | 58 | 74 | 79 |
| | 3.4 | 1.9 | −4.2 | 14 | 9.6 | 33 | 45 | 62 | 74 | 82 |
| | 6.8 | 12 | 12 | 17 | 14 | 37 | 46 | 56 | 74 | 76 |
| | 14 | 27 | 42 | 43 | 42 | 53 | 64 | 68 | 85 | 68 |
| | 27 | 62 | 57 | 57 | 64 | 67 | 71 | 71 | 73 | 81 |

TABLE 28

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Cyproheptadine HCl (μM) | 0 | −5.9 | −4.3 | −7.7 | 3.4 | 14 | 31 | 59 | 82 | 94 |
| | 0.24 | 0.99 | 0.47 | 3.4 | 3 | 14 | 39 | 62 | 81 | 56 |
| | 0.48 | −2.8 | 2.7 | 1.3 | 6.2 | 19 | 42 | 62 | 83 | 90 |
| | 0.96 | 7 | 3.3 | 5.5 | 8.6 | 18 | 48 | 65 | 83 | 83 |
| | 1.9 | 9.3 | 8.8 | 14 | 18 | 28 | 46 | 68 | 81 | 87 |
| | 3.9 | 21 | 17 | 26 | 26 | 36 | 55 | 69 | 84 | 83 |
| | 7.7 | 36 | 39 | 37 | 41 | 51 | 66 | 74 | 85 | 94 |
| | 15 | 58 | 64 | 62 | 65 | 70 | 77 | 82 | 86 | 61 |
| | 31 | 82 | 82 | 82 | 82 | 86 | 88 | 83 | 93 | 72 |

TABLE 29

| | | Bromodiphenhydramine HCl (μM) | | | | | | | | |
|---|---|---|---|---|---|---|---|---|---|---|
| | | 0 | 0.21 | 0.43 | 0.85 | 1.7 | 3.4 | 6.8 | 14 | 27 |
| Fluoxetine HCl (μM) | 0 | 0.35 | −0.352 | 5.627 | 4.473 | 9.707 | 13.74 | 17.18 | 38.98 | 65.22 |
| | 0.23 | 5.748 | 6.455 | 7.335 | 7.771 | 8.58 | 13.38 | 21.4 | 41.7 | 66.45 |
| | 0.45 | 5.793 | 9.625 | 7.67 | 7.37 | 8.547 | 15.26 | 26.43 | 42.98 | 69.7 |
| | 0.9 | 5.52 | 5.315 | 11.11 | 15.88 | 9.732 | 16.45 | 26.48 | 42.08 | 70.35 |
| | 1.8 | 5.42 | 6.611 | 10.45 | 18.03 | 14.23 | 15.91 | 27.77 | 38.35 | 68.55 |
| | 3.6 | 16.67 | 22.38 | 19.39 | 21.48 | 28.57 | 26.43 | 35.73 | 53.5 | 69.15 |
| | 7.2 | 34.88 | 41.83 | 43.25 | 47.08 | 47.3 | 52.12 | 54.15 | 62.75 | 76.8 |
| | 14 | 64.47 | 69.25 | 64.4 | 66.55 | 67.03 | 68.8 | 67.7 | 75.47 | 80.88 |
| | 29 | 71.5 | 76.7 | 74.92 | 76.53 | 72.8 | 74.08 | 75.25 | 86.22 | 88.18 |

TABLE 30

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Fluoxetine HCl (μM) | 0 | −1.285 | 2.175 | −2.466 | 8.947 | 6.506 | 4.911 | 7.435 | 26.05 | 34.1 |
| | 0.23 | 0.012 | 1.759 | −1.4 | 1.025 | 7.708 | 5.717 | 12.4 | 22.1 | 38.72 |
| | 0.45 | −0.332 | 3.74 | 1.932 | 5.771 | 3.794 | 7.753 | 12.52 | 25.5 | 41.1 |
| | 0.9 | −6.572 | 2.343 | 2.815 | 4.072 | 2.358 | 8.643 | 10.11 | 27.62 | 42.8 |
| | 1.8 | −1.713 | 0.7252 | 0.8725 | 7.713 | 10.46 | 13.68 | 18.18 | 29.58 | 44.12 |
| | 3.6 | 15.77 | 15.73 | 20.45 | 10 | 22.85 | 21.65 | 29.95 | 35.82 | 53.07 |
| | 7.2 | 32.55 | 37.15 | 37.98 | 37.17 | 39.97 | 45.22 | 48.65 | 51.8 | 62.38 |
| | 14 | 61.65 | 64.58 | 65.6 | 64.9 | 67 | 65.55 | 67 | 71.58 | 76.75 |
| | 29 | 63.88 | 68.03 | 63.45 | 72.18 | 79.52 | 76.55 | 75.85 | 83.12 | 84.97 |

TABLE 31

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Fluoxetine HCl (μM) | 0 | −4.192 | 3.84 | 5.832 | 12.21 | 21.55 | 32.58 | 51.02 | 76.35 | 86.65 |
| | 0.23 | −0.122 | 5.882 | 4.968 | 13 | 17.57 | 31.35 | 54.88 | 80.8 | 91.78 |
| | 0.45 | −2.697 | 0.3675 | 10.17 | 11.66 | 14.11 | 29.88 | 53.85 | 80.67 | 93.85 |
| | 0.9 | −1.265 | 6.242 | 8.24 | 13.5 | 20.75 | 32.88 | 58.43 | 80.83 | 93.47 |
| | 1.8 | 2.228 | 7.982 | 9.643 | 16.12 | 22.98 | 34.92 | 60.62 | 82.62 | 92.35 |
| | 3.6 | 16.12 | 18.88 | 22.73 | 22.27 | 32.23 | 42.43 | 66.82 | 86 | 90.93 |
| | 7.2 | 33.5 | 40.22 | 42.9 | 47.15 | 51 | 60.95 | 74.2 | 87.82 | 94.12 |
| | 14 | 64.68 | 70.15 | 68.88 | 70.92 | 72.97 | 78.08 | 84.07 | 91.68 | 92.17 |
| | 29 | 85.35 | 84.9 | 86.85 | 88.93 | 89.72 | 91.75 | 91.75 | 93.48 | 94.45 |

TABLE 32

| | | Loratadine (μM) | | | | | | | | |
|---|---|---|---|---|---|---|---|---|---|---|
| | | 0 | 0.2 | 0.41 | 0.82 | 1.6 | 3.3 | 6.5 | 13 | 26 |
| Fluoxetine HCl (μM) | 0 | −1.205 | 3.68 | 4.647 | 4.407 | 7.09 | 13.56 | 22.95 | 42.92 | 56.62 |
| | 0.23 | −1.193 | 3.64 | 1.047 | 2.758 | 9.598 | 14.75 | 26.7 | 43.8 | 61.5 |
| | 0.45 | −2.74 | −1.694 | 1.695 | 7.343 | 3.063 | 16.86 | 26.78 | 42.9 | 62.75 |
| | 0.9 | 0.9675 | 1.278 | 2.23 | 4.625 | 12.8 | 18.6 | 33.12 | 45.47 | 60.9 |
| | 1.8 | 4.62 | 3.707 | 7.85 | 9.148 | 18 | 25.05 | 37.48 | 53.2 | 67.42 |
| | 3.6 | 13.73 | 15.05 | 22.83 | 24.75 | 33.15 | 39.62 | 52.42 | 63.75 | 74.38 |
| | 7.2 | 32.3 | 44.65 | 43.05 | 48.38 | 54.27 | 62.65 | 67.5 | 71.05 | 81.35 |
| | 14 | 68.1 | 64.75 | 67.82 | 68.67 | 73.1 | 76.92 | 76.72 | 81.52 | 85.78 |
| | 29 | 83.05 | 81.42 | 84.67 | 87 | 84.88 | 87.65 | 86.07 | 88.1 | 88.25 |

TABLE 33

| | | Thiethylperazine Maleate (μM) | | | | | | | | |
|---|---|---|---|---|---|---|---|---|---|---|
| | | 0 | 0.2 | 0.39 | 0.78 | 1.6 | 3.1 | 6.3 | 13 | 25 |
| Fluoxetine HCl (μM) | 0 | 0.837 | −0.065 | −2.473 | −1.133 | 2.398 | 4.31 | 17.7 | 55.22 | 82.28 |
| | 0.23 | 1.883 | 3.75 | 3.323 | 6.375 | 4.215 | 6.143 | 20.78 | 60.22 | 83.73 |
| | 0.45 | −0.58 | 1.214 | 3.86 | 7.471 | 1.661 | 4.84 | 20.45 | 59.93 | 85.55 |
| | 0.9 | 1.545 | −1.533 | −1.003 | 2.783 | 4.505 | 7.848 | 32.42 | 62.3 | 87.03 |
| | 1.8 | 0.562 | −5.368 | 0.317 | 4.005 | 3.777 | 8.165 | 34.85 | 57.5 | 85.45 |
| | 3.6 | 6.117 | 18.9 | 18.9 | 22.12 | 21 | 29.85 | 43.33 | 65.97 | 84.75 |
| | 7.2 | 37.12 | 37.7 | 40.55 | 44.98 | 42.25 | 49.25 | 58.65 | 75.53 | 89.9 |
| | 14 | 61.05 | 65.85 | 63.7 | 65.23 | 66.97 | 67.65 | 74.42 | 82.58 | 90.08 |
| | 29 | 61.05 | 65.85 | 63.7 | 65.23 | 66.97 | 67.65 | 74.42 | 82.58 | 85.4 |

TABLE 34

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Sertraline HCl (μM) | 0 | −3.043 | 0.815 | 0.655 | 3.827 | 5.165 | 6.897 | 13.93 | 24.47 | 43.3 |
| | 0.25 | 2.633 | 0.5175 | 6.315 | 9.397 | 7.032 | 7.282 | 11.74 | 26.65 | 47.4 |
| | 0.49 | 1.905 | 5.773 | 3.93 | −0.112 | 10.12 | 8.317 | 15.25 | 23.8 | 48.27 |
| | 0.99 | 8.723 | 10.38 | 6.08 | 9.207 | 8.455 | 14.44 | 18 | 32.1 | 53.25 |
| | 2 | 8.373 | 11.81 | 3.715 | 11.69 | 9.878 | 18.3 | 28.1 | 37.3 | 54.55 |
| | 4 | 24.2 | 24.32 | 18.29 | 24.45 | 29.05 | 35.92 | 41.75 | 55.2 | 66.88 |
| | 7.9 | 58.85 | 55.27 | 53.85 | 51.78 | 64.67 | 64.12 | 67.6 | 75.45 | 80.9 |
| | 16 | 82.1 | 82.53 | 79.38 | 84.27 | 85.45 | 86.17 | 85.42 | 88.72 | 86.83 |
| | 32 | 92.3 | 90.62 | 91.1 | 87.12 | 92.75 | 90.38 | 91.75 | 89.67 | 93.57 |

TABLE 35

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Sertraline HCl (μM) | 0 | −0.322 | 2.54 | 9.672 | 6.178 | 14.07 | 28.9 | 45.3 | 71.18 | 87.6 |
| | 0.25 | 0.615 | 2.368 | 0.875 | 8.447 | 11.83 | 25.18 | 44.2 | 73 | 83.9 |
| | 0.5 | −3.708 | 6.518 | 7.16 | 7.425 | 10.15 | 29.9 | 43.43 | 74.28 | 88.33 |
| | 1 | 4.73 | 6.124 | 4.436 | 7.862 | 15.46 | 28.6 | 52.8 | 76.22 | 86.45 |
| | 2 | 8.768 | 8.80 | 7.782 | 11.3 | 20.02 | 36.6 | 52.1 | 73.1 | 86.97 |
| | 4 | 22.09 | 27.77 | 29.25 | 23.58 | 33.65 | 44.9 | 59.27 | 76.97 | 85.88 |
| | 7.9 | 57.9 | 53.5 | 59.35 | 57.2 | 63.12 | 67.9 | 71.8 | 84.57 | 89.6 |
| | 16 | 77.4 | 79.9 | 74.95 | 76.45 | 81.72 | 80.12 | 80.88 | 87.62 | 89.08 |
| | 32 | 84.55 | 84.5 | 83.62 | 79.85 | 86.65 | 81.22 | 88.38 | 84.5 | 89.1 |

TABLE 36

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Sertraline HCl (μM) | 0 | −2.448 | −1.755 | 0.275 | −1.147 | 0.285 | 5.617 | 16.8 | 40.05 | 72.1 |
| | 0.25 | 2.83 | 1.935 | 1.37 | 0.511 | 1.427 | 4.865 | 19.02 | 39.85 | 72.1 |
| | 0.5 | 0.67 | 3.755 | 0.442 | 1.647 | 0.297 | 3.17 | 18.9 | 45.12 | 73.03 |
| | 1 | −0.696 | −1.335 | 5.095 | 1.795 | 3.662 | 9.712 | 24.67 | 47.55 | 74 |
| | 2 | 3.635 | 7.189 | 5.68 | 5.28 | 7.625 | 17.05 | 27.88 | 50.9 | 75.3 |
| | 4 | 23.55 | 22.8 | 27.15 | 24.3 | 22.55 | 34.2 | 44.67 | 61.88 | 78.17 |
| | 7.9 | 51.22 | 51.57 | 52.95 | 58.83 | 56.43 | 59.2 | 68.42 | 76.78 | 86.35 |
| | 16 | 81.85 | 83.65 | 83 | 81.45 | 82 | 82.45 | 85.25 | 86.03 | 87.7 |
| | 32 | 90.35 | 90.07 | 90.17 | 91.2 | 89.57 | 89.55 | 91.35 | 90.17 | 92.32 |

TABLE 37

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Desipramine HCl (μM) | 0 | −3.209 | 2.63 | 9.587 | 10.12 | 21.72 | 36.7 | 50.83 | 73.38 | 87.05 |
| | 0.25 | −0.153 | 3.443 | 1.526 | 11.65 | 16.27 | 30.8 | 52.15 | 74.6 | 91.47 |
| | 0.5 | −5.603 | 2.725 | 4.099 | 10.06 | 20.48 | 29.22 | 46.55 | 76.62 | 91.83 |
| | 1 | 5.153 | 8.555 | 4.983 | 12.38 | 18.95 | 30.12 | 54.92 | 78.62 | 92.2 |
| | 2 | 1.947 | 3.468 | 5.553 | 11.76 | 18.8 | 32.15 | 51.85 | 73.83 | 92 |
| | 4 | 12.65 | 10.76 | 12.67 | 21.3 | 21.98 | 37.75 | 53.38 | 78.8 | 91.33 |
| | 8 | 31.1 | 36.2 | 39.05 | 41.1 | 44.8 | 57.08 | 62.1 | 85.85 | 94.17 |
| | 16 | 58.07 | 57.85 | 60.22 | 63.43 | 67.5 | 71.35 | 82.22 | 87.18 | 92.1 |
| | 32 | 79.17 | 82.1 | 80.2 | 83.9 | 85.97 | 85.58 | 89.28 | 90 | 92.5 |

TABLE 38

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Desipramine HCl (μM) | 0 | −3.611 | 0.332 | 2.81 | −0.438 | 0.46 | 3.7 | 22.47 | 42.8 | 75.65 |
| | 0.25 | 6.397 | 5.445 | −0.25 | 3.098 | 2.806 | 7.095 | 16.88 | 44.03 | 73.17 |
| | 0.5 | 0.621 | 2.451 | 0.467 | 2.876 | 1.265 | 6.543 | 19.85 | 42.2 | 74.42 |
| | 1 | 9.88 | 8.752 | 2.875 | −3.072 | −0.121 | 12.57 | 24.7 | 45.92 | 77.88 |
| | 2 | 4.349 | 3.4 | 4.31 | 0.5375 | 0.89 | 15.95 | 21.72 | 42.45 | 74.55 |
| | 4 | 12.41 | 8.735 | 12.12 | 11.73 | 13.04 | 25.55 | 30.58 | 52.48 | 78.03 |
| | 8 | 34.05 | 32.4 | 36.4 | 34.78 | 36.35 | 44.5 | 50 | 67.53 | 83.12 |
| | 16 | 55.98 | 54.02 | 58.15 | 61.05 | 63.05 | 61.23 | 69.57 | 77.58 | 85.57 |
| | 32 | 80.83 | 80.72 | 79.15 | 82.88 | 81.33 | 80.55 | 82.2 | 89.28 | 87.45 |

TABLE 39

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Desipramine HCl (μM) | 0 | −0.527 | −1.995 | 2.296 | 5.108 | −0.825 | 1.697 | 5.553 | 26.43 | 41.43 |
| | 0.25 | 4.761 | 7.332 | −0.690 | 0.225 | 2.35 | 3.275 | 10.61 | 27.3 | 49.65 |
| | 0.5 | −1.125 | −3.788 | 0.639 | −2.614 | −0.809 | −2.265 | 5.592 | 18.25 | 43.93 |
| | 1 | 6.13 | 8.043 | 4.515 | 2.828 | −0.423 | 2.98 | 14.48 | 27.27 | 43.25 |
| | 2 | −2.047 | −3.89 | −1.72 | 0.212 | 5.305 | 5.953 | 9.032 | 25.22 | 46.53 |
| | 4 | 7.088 | 13.58 | 6.583 | 8.96 | 13.95 | 14.01 | 21.18 | 33.55 | 56.65 |
| | 8 | 25.1 | 23.63 | 24.25 | 25.35 | 34.55 | 39.83 | 51.9 | 57.12 | 71.97 |
| | 16 | 60 | 60.9 | 59.32 | 62.9 | 61.45 | 63.73 | 71.2 | 78.33 | 86.77 |
| | 32 | 85.67 | 85.1 | 84.92 | 85.38 | 87.15 | 85.43 | 86.97 | 91.25 | 93.5 |

TABLE 40

| | | Loratadine (μM) | | | | | | | | |
|---|---|---|---|---|---|---|---|---|---|---|
| | | 0 | 0.2 | 0.41 | 0.82 | 1.6 | 3.3 | 6.5 | 13 | 26 |
| Desipramine HCl (μM) | 0 | −1.78 | −1.949 | 2.137 | 7.773 | 8.037 | 12.41 | 16.46 | 31.7 | 41.48 |
| | 0.25 | 1.777 | 6.125 | 2.318 | 4.715 | 12.17 | 17.15 | 24.73 | 32 | 54.72 |
| | 0.5 | −1.765 | 5.99 | 0.9765 | 8.04 | 13.06 | 17.93 | 25.45 | 36.95 | 57.15 |
| | 1 | 0.8395 | −1.757 | 4.155 | 3.262 | 12.48 | 16.18 | 26.02 | 39.85 | 55.37 |
| | 2 | −2.28 | 8.093 | 10.5 | 8.999 | 14.45 | 25.38 | 31.12 | 38.2 | 59.88 |
| | 4 | 8.5 | 17.3 | 11.63 | 17.29 | 24.82 | 38.55 | 39.2 | 53.1 | 71.5 |
| | 8 | 26.82 | 29.88 | 30.98 | 33.92 | 42.35 | 56.25 | 58.08 | 69.22 | 78.32 |
| | 16 | 54 | 58.22 | 58.55 | 58.58 | 64.85 | 71.82 | 72.1 | 82.02 | 85.75 |
| | 32 | 78.33 | 75.7 | 80.32 | 81.12 | 82.8 | 85.9 | 82.8 | 87.1 | 80.05 |

TABLE 41

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Amoxapine (μM) | 0 | −4.37 | 3.84 | 0.737 | 7.52 | 18.4 | 36.3 | 60.2 | 83.8 | 94 |
| | 0.2 | 2.44 | 7.82 | 7.93 | 13 | 23.9 | 39 | 61.8 | 85.8 | 95.2 |
| | 0.4 | 7.03 | 12.5 | 12.1 | 16.9 | 25.2 | 42 | 64.1 | 86.7 | 93.3 |
| | 0.8 | 18.4 | 22.4 | 25.2 | 25.9 | 35.2 | 47.9 | 67.3 | 87.4 | 92.7 |
| | 1.6 | 32.5 | 37.1 | 39.6 | 41.3 | 48.7 | 59.8 | 72.5 | 87.1 | 93.5 |
| | 3.2 | 50.3 | 53.3 | 52.1 | 56.9 | 60.5 | 69 | 78.4 | 89.9 | 94.6 |
| | 6.4 | 67.4 | 71.9 | 71.6 | 73.8 | 75.9 | 78.9 | 85.8 | 91.2 | 95.4 |
| | 13 | 85.3 | 87.3 | 83.9 | 85.7 | 87.8 | 89.4 | 91.7 | 94.6 | 94.6 |
| | 25 | 93.5 | 95.4 | 94.5 | 94.2 | 95 | 93.5 | 95.3 | 96.1 | 94.8 |

TABLE 42

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Nortriptyline HCl (μM) | 0 | −7.07 | 2.67 | 4.28 | 10 | 19.2 | 35.1 | 54.9 | 77.6 | 89.4 |
| | 0.26 | −0.273 | 2.83 | 7.59 | 11.4 | 25 | 35.8 | 55.9 | 78.9 | 90.8 |
| | 0.52 | −2.72 | 3.05 | 5.37 | 13.5 | 21.2 | 34.7 | 59.5 | 78.6 | 88.9 |
| | 1 | 2.88 | 8.52 | 12.1 | 13.9 | 22.9 | 40.3 | 61.6 | 82.3 | 90.1 |
| | 2.1 | 11.1 | 18.1 | 20.3 | 26 | 34.6 | 48.6 | 66.3 | 83.9 | 91.1 |
| | 4.2 | 31.7 | 38.4 | 36.2 | 41.7 | 47.3 | 60 | 72.6 | 84.7 | 89.5 |
| | 8.3 | 54.8 | 59 | 58.9 | 63.5 | 66.8 | 73.2 | 79.2 | 85.3 | 88.9 |
| | 17 | 77.2 | 80.4 | 79.2 | 81.8 | 83 | 85.3 | 87.2 | 89 | 91.3 |
| | 33 | 87.5 | 87.9 | 88.7 | 87.3 | 91.5 | 89.2 | 89 | 90.1 | 91.4 |

TABLE 43

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Paroxetine HCl (μM) | 0 | −1.97 | 1.95 | 1.08 | 14.3 | 17.9 | 34.9 | 58.6 | 83.4 | 92.2 |
| | 0.21 | −1.21 | 3.59 | 6.21 | 15.5 | 25.3 | 38.3 | 63.2 | 85.8 | 92.6 |
| | 0.42 | −0.362 | 6.48 | 8.24 | 15.4 | 25.2 | 40.3 | 64.7 | 88.9 | 93.8 |
| | 0.83 | 4.32 | 10.8 | 15 | 23.2 | 32.7 | 50 | 68.7 | 87.5 | 92 |
| | 1.7 | 20.6 | 24.1 | 30.6 | 36.8 | 43.7 | 56.2 | 73.3 | 89.2 | 94.3 |
| | 3.3 | 42.6 | 46.2 | 42.5 | 49.9 | 57.8 | 69 | 78.4 | 91.1 | 92.4 |
| | 6.7 | 63.4 | 66.2 | 69 | 71.9 | 76.4 | 80 | 87.2 | 93.6 | 93.7 |
| | 13 | 86.3 | 88 | 90.1 | 89.3 | 90.4 | 91 | 92.9 | 92.4 | 95.7 |
| | 27 | 91.4 | 91.1 | 94.8 | 94.2 | 95.9 | 92.9 | 94.7 | 93.8 | 93.4 |

TABLE 44

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Amoxapine (μM) | 0 | −4.37 | 3.84 | 0.737 | 7.52 | 18.4 | 36.3 | 60.2 | 83.8 | 94 |
| | 0.2 | 2.44 | 7.82 | 7.93 | 13 | 23.9 | 39 | 61.8 | 85.8 | 95.2 |
| | 0.4 | 7.03 | 12.5 | 12.1 | 16.9 | 25.2 | 42 | 64.1 | 86.7 | 93.3 |
| | 0.8 | 18.4 | 22.4 | 25.2 | 25.9 | 35.2 | 47.9 | 67.3 | 87.4 | 92.7 |
| | 1.6 | 32.5 | 37.1 | 39.6 | 41.3 | 48.7 | 59.8 | 72.5 | 87.1 | 93.5 |
| | 3.2 | 50.3 | 53.3 | 52.1 | 56.9 | 60.5 | 69 | 78.4 | 89.9 | 94.6 |
| | 6.4 | 67.4 | 71.9 | 71.6 | 73.8 | 75.9 | 78.9 | 85.8 | 91.2 | 95.4 |
| | 13 | 85.3 | 87.3 | 83.9 | 85.7 | 87.8 | 89.4 | 91.7 | 94.6 | 94.6 |
| | 25 | 93.5 | 95.4 | 94.5 | 94.2 | 95 | 93.5 | 95.3 | 96.1 | 94.8 |

TABLE 45

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Loratadine (μM) | 0 | −3.51 | 7.52 | 11.2 | 22.2 | 33.2 | 50.1 | 64.3 | 83.7 | 88.2 |
| | 0.2 | 0.269 | 11.4 | 13 | 23.1 | 37.1 | 52.5 | 68.6 | 85.1 | 92.4 |
| | 0.41 | 2.44 | 10.9 | 12.6 | 27.5 | 39.2 | 55 | 68.1 | 83.7 | 93 |
| | 0.82 | 1.28 | 15.7 | 18.5 | 29.5 | 42.4 | 58.1 | 71.3 | 85.1 | 92.8 |
| | 1.6 | 8.07 | 21.5 | 26.4 | 36.5 | 49.2 | 64 | 74.9 | 86.6 | 92.5 |
| | 3.3 | 17.1 | 29.3 | 36.4 | 45.6 | 56.7 | 68.9 | 78 | 88 | 93.5 |
| | 6.5 | 27.7 | 42.3 | 45.4 | 57.4 | 64.7 | 72.5 | 82.1 | 88.8 | 94.1 |
| | 13 | 46.3 | 61 | 62.6 | 68.7 | 74.6 | 81.3 | 85.3 | 92.2 | 92.8 |
| | 26 | 64.7 | 74 | 76.1 | 79 | 81.9 | 86.5 | 90.2 | 91.5 | 93 |

TABLE 46

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Loratadine (μM) | 0 | −2.34 | 2.29 | 4.76 | 6.88 | 19.3 | 33.5 | 60.3 | 80.8 | 90.9 |
| | 0.2 | 2.49 | 2.77 | 4.15 | 14.5 | 21.7 | 38.2 | 63.3 | 83.9 | 92 |
| | 0.41 | 0.369 | 7.05 | 5.29 | 14.3 | 21 | 42.4 | 64.8 | 81.8 | 93.5 |
| | 0.82 | 6.29 | 10.2 | 13.1 | 19.4 | 30.5 | 46.7 | 68 | 84.1 | 93.6 |
| | 1.6 | 9 | 12.7 | 15.6 | 23.1 | 38.4 | 53.4 | 71.4 | 86.3 | 94.1 |
| | 3.3 | 14.2 | 22.9 | 26.4 | 34.2 | 46.2 | 62.4 | 76.3 | 87.5 | 93.6 |
| | 6.5 | 26.8 | 32.2 | 37.4 | 48 | 56.5 | 70.4 | 81 | 88.5 | 93.5 |
| | 13 | 39.9 | 50.3 | 52.8 | 61.3 | 68 | 77.6 | 85.5 | 91.1 | 94.8 |
| | 26 | 58.9 | 64.9 | 66.4 | 69.8 | 75.4 | 83.2 | 88.7 | 93 | 93.2 |

TABLE 47

| | | Loratadine (μM) | | | | | | | | |
|---|---|---|---|---|---|---|---|---|---|---|
| | | 0 | 0.2 | 0.41 | 0.82 | 1.6 | 3.3 | 6.5 | 13 | 26 |
| Paroxetine HCl (μM) | 0 | −5.06 | −1.38 | 1.24 | 1.77 | 7.81 | 10.9 | 20.5 | 38.3 | 43.4 |
| | 0.21 | 0.381 | 0.773 | 1.61 | 7.37 | 9.53 | 14 | 26.2 | 42.9 | 52.6 |
| | 0.42 | 3.62 | −1.25 | −0.921 | 4.12 | 6.17 | 22.4 | 32.1 | 46.1 | 62 |
| | 0.83 | 7.02 | 9.21 | 10.5 | 10.3 | 17.3 | 28.3 | 40.2 | 56.4 | 62.5 |
| | 1.7 | 12.2 | 18.9 | 14.9 | 23.1 | 30 | 41.2 | 50 | 64.6 | 73.4 |
| | 3.3 | 32.9 | 38.2 | 35.5 | 45.4 | 52.7 | 63.3 | 67.7 | 78.2 | 83.2 |
| | 6.7 | 63.8 | 62.9 | 66.6 | 69.5 | 72.3 | 75.9 | 79.6 | 84 | 89.6 |
| | 13 | 83.8 | 83.7 | 82.9 | 85 | 87.1 | 88.6 | 90 | 90.9 | 94.2 |
| | 27 | 93.8 | 94.9 | 93.6 | 93.9 | 95.7 | 94.9 | 94.1 | 95.9 | 94.9 |

TABLE 48

| | | Fluoxetine HCl (μM) | | | | | | | | |
|---|---|---|---|---|---|---|---|---|---|---|
| | | 0 | 0.23 | 0.45 | 0.9 | 1.8 | 3.6 | 7.2 | 14 | 29 |
| Cyproheptadine HCl (μM) | 0 | −2.2 | −0.76 | −3.5 | −1.1 | −0.65 | −2.3 | 4.1 | 16 | 41 |
| | 0.24 | −1.4 | 2.2 | 5.4 | 2.7 | 15 | 6.7 | 6.9 | 20 | 26 |
| | 0.48 | −2.7 | 2.4 | 0.2 | 1.8 | 3 | 6.3 | 3.9 | 20 | 41 |
| | 0.96 | 6 | 4.3 | 3 | 9.3 | 10 | 13 | 15 | 24 | 47 |
| | 1.9 | 11 | 13 | 19 | 13 | 14 | 17 | 21 | 32 | 50 |
| | 3.9 | 18 | 26 | 25 | 29 | 24 | 29 | 34 | 38 | 55 |
| | 7.7 | 40 | 43 | 44 | 40 | 43 | 56 | 52 | 59 | 66 |
| | 15 | 63 | 67 | 64 | 66 | 71 | 68 | 70 | 72 | 75 |
| | 31 | 79 | 83 | 81 | 86 | 85 | 85 | 85 | 84 | 88 |

TABLE 49

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Azatadine Maleate (μM) | 0 | −3.1 | 1.7 | 12 | 12 | 25 | 31 | 38 | 35 | 43 |
| | 0.27 | 1.2 | 1.2 | 11 | 16 | 25 | 26 | 36 | 35 | 38 |
| | 0.54 | −13 | −2 | 8.3 | 10 | 24 | 30 | 29 | 39 | 43 |
| | 1.1 | −7.7 | −0.84 | 6 | 13 | 20 | 24 | 28 | 37 | 36 |
| | 2.1 | −6.7 | 0.76 | 11 | 9 | 18 | 25 | 27 | 36 | 40 |
| | 4.3 | −0.76 | 10 | 15 | 8.6 | 18 | 26 | 32 | 35 | 37 |
| | 8.6 | −1.6 | 1.5 | 8.2 | 12 | 24 | 32 | 34 | 33 | 40 |
| | 17 | 9.1 | 9.7 | 14 | 20 | 27 | 38 | 32 | 42 | 42 |
| | 34 | 17 | 29 | 28 | 29 | 37 | 43 | 42 | 50 | 51 |

TABLE 50

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Bromodiphenhydramine | 0 | 7.9 | 0.3 | 6.3 | 17 | 22 | 34 | 37 | 34 | 37 |
| HCl (μM) | 0.21 | −0.64 | 6.6 | 5.9 | 17 | 24 | 30 | 31 | 34 | 40 |
| | 0.43 | −7 | −0.23 | 2.5 | 18 | 23 | 28 | 35 | 37 | 33 |
| | 0.85 | −3.4 | 2.7 | 6.1 | 21 | 33 | 33 | 36 | 40 | 36 |
| | 1.7 | −7 | 2.7 | 8.8 | 28 | 28 | 29 | 34 | 39 | 34 |
| | 3.4 | 2.7 | 18 | 17 | 25 | 30 | 31 | 34 | 34 | 35 |
| | 6.8 | 23 | 33 | 33 | 37 | 43 | 46 | 52 | 56 | 56 |
| | 14 | 46 | 51 | 59 | 56 | 60 | 67 | 67 | 67 | 65 |
| | 27 | 66 | 66 | 74 | 77 | 79 | 83 | 80 | 83 | 80 |

TABLE 51

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Cetrizine HCl (μM) | 0 | 13 | 11 | 11 | 23 | 26 | 40 | 37 | 39 | 40 |
| | 0.2 | 0.93 | 16 | 22 | 27 | 32 | 38 | 39 | 40 | 20 |
| | 0.4 | 2.4 | 15 | 12 | 29 | 33 | 36 | 42 | 42 | 42 |
| | 0.8 | 2.4 | 19 | 22 | 29 | 31 | 40 | 42 | 39 | 41 |
| | 1.6 | 4.2 | 17 | 22 | 32 | 30 | 38 | 41 | 41 | 41 |
| | 3.2 | 14 | 15 | 18 | 28 | 32 | 39 | 43 | 42 | 45 |
| | 6.4 | 5.5 | 19 | 20 | 29 | 36 | 40 | 41 | 45 | 43 |
| | 13 | 7 | 19 | 20 | 29 | 35 | 39 | 41 | 42 | 47 |
| | 26 | 11 | 21 | 24 | 30 | 37 | 43 | 42 | 46 | 47 |

TABLE 52

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Chlorpheniramine Maleate | 0 | 0.041 | 0.65 | 9 | 22 | 27 | 33 | 37 | 28 | 39 |
| (μM) | 0.2 | −3.9 | 5.3 | 9.2 | 16 | 28 | 33 | 32 | 35 | 34 |
| | 0.4 | 0.5 | −2.1 | 6.7 | 18 | 25 | 26 | 32 | 37 | 35 |
| | 0.8 | −5.7 | 1.7 | 12 | 16 | 30 | 28 | 32 | 38 | 41 |
| | 1.6 | −4.6 | −1.8 | 9 | 16 | 25 | 26 | 29 | 34 | 34 |
| | 3.2 | −3.1 | 3.5 | 7.6 | 18 | 24 | 28 | 36 | 32 | 35 |
| | 6.4 | 5.8 | 9.7 | 15 | 22 | 30 | 32 | 36 | 39 | 41 |
| | 13 | 7.1 | 22 | 28 | 27 | 37 | 48 | 42 | 45 | 49 |
| | 26 | 25 | 31 | 34 | 37 | 45 | 50 | 50 | 54 | 52 |

TABLE 53

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Clemizole HCl (μM) | 0 | 1.1 | 3.9 | 8.8 | 17 | 21 | 31 | 35 | 37 | 40 |
| | 0.22 | −5.6 | 4.6 | 11 | 16 | 25 | 28 | 33 | 38 | 36 |
| | 0.43 | −0.58 | 6.2 | 10 | 18 | 26 | 30 | 38 | 37 | 39 |
| | 0.86 | −3.2 | 3.9 | 11 | 21 | 28 | 32 | 36 | 36 | 36 |
| | 1.7 | −0.19 | 3.4 | 13 | 24 | 28 | 34 | 34 | 35 | 34 |
| | 3.5 | 3.2 | 5.9 | 17 | 22 | 28 | 36 | 35 | 33 | 38 |
| | 6.9 | 14 | 22 | 23 | 31 | 39 | 42 | 47 | 44 | 45 |
| | 14 | 30 | 32 | 33 | 46 | 46 | 54 | 56 | 57 | 54 |
| | 28 | 44 | 56 | 54 | 56 | 62 | 66 | 65 | 70 | 68 |

TABLE 54

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Cyproheptadine HCl (μM) | 0 | −5.3 | 13 | 13 | 22 | 29 | 32 | 34 | 39 | 39 |
| | 0.24 | −0.44 | 14 | 20 | 24 | 30 | 39 | 38 | 42 | 42 |
| | 0.48 | 3.2 | 13 | 17 | 23 | 29 | 34 | 40 | 38 | 40 |
| | 0.96 | 0.41 | 13 | 23 | 30 | 30 | 42 | 40 | 42 | 41 |
| | 1.9 | 12 | 18 | 25 | 33 | 35 | 44 | 41 | 44 | 44 |
| | 3.9 | 14 | 31 | 34 | 41 | 45 | 46 | 47 | 52 | 53 |
| | 7.7 | 32 | 42 | 39 | 50 | 57 | 57 | 61 | 62 | 60 |
| | 15 | 54 | 62 | 61 | 67 | 67 | 71 | 71 | 70 | 72 |
| | 31 | 77 | 80 | 80 | 84 | 84 | 85 | 86 | 85 | 87 |

TABLE 55

| | | Prednisolone (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Desloratadine (μM) | 0 | −6.2 | 1.4 | 4.2 | 13 | 20 | 26 | 30 | 35 | 37 |
| | 0.25 | −1.3 | −0.14 | 4.7 | 16 | 19 | 24 | 31 | 32 | 38 |
| | 0.51 | −2.7 | 4.4 | 10 | 16 | 23 | 29 | 34 | 35 | 38 |
| | 1 | 6.6 | 0.94 | 14 | 13 | 25 | 27 | 35 | 37 | 42 |
| | 2 | 16 | 22 | 24 | 29 | 34 | 38 | 42 | 48 | 49 |
| | 4.1 | 30 | 31 | 35 | 42 | 42 | 50 | 53 | 54 | 60 |
| | 8.1 | 58 | 63 | 62 | 65 | 68 | 73 | 73 | 76 | 77 |
| | 16 | 86 | 88 | 88 | 88 | 89 | 91 | 91 | 92 | 92 |
| | 33 | 96 | 96 | 97 | 97 | 97 | 97 | 96 | 97 | 97 |

TABLE 56

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Dimenhydrinate (μM) | 0 | −0.83 | 11 | 5.4 | 17 | 19 | 27 | 26 | 32 | 34 |
| | 0.17 | −3.2 | 10 | 9.7 | 20 | 23 | 28 | 30 | 35 | 34 |
| | 0.33 | −0.92 | 6.4 | 11 | 16 | 28 | 29 | 32 | 35 | 35 |
| | 0.66 | −0.85 | 11 | 14 | 20 | 28 | 32 | 30 | 31 | 33 |
| | 1.3 | 0.47 | 12 | 17 | 21 | 26 | 28 | 35 | 39 | 39 |
| | 2.7 | 4 | 11 | 16 | 26 | 27 | 33 | 37 | 35 | 37 |
| | 5.3 | 6.2 | 16 | 17 | 27 | 30 | 33 | 37 | 38 | 41 |
| | 11 | 13 | 23 | 23 | 30 | 32 | 38 | 42 | 39 | 43 |
| | 21 | 17 | 26 | 29 | 40 | 41 | 43 | 43 | 47 | 50 |

TABLE 57

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Doxylamine Succinate | 0 | 1.7 | 16 | 17 | 20 | 25 | 33 | 31 | 32 | 35 |
| (μM) | 0.2 | 3.5 | 9.4 | 13 | 21 | 24 | 30 | 31 | 34 | 34 |
| | 0.4 | 0.96 | 11 | 15 | 20 | 29 | 29 | 34 | 31 | 34 |
| | 0.8 | −9.1 | 10 | 9.6 | 21 | 27 | 28 | 28 | 30 | 32 |
| | 1.6 | 3.3 | 8.5 | 15 | 19 | 25 | 30 | 31 | 33 | 36 |
| | 3.2 | 9.1 | 9.5 | 17 | 20 | 23 | 30 | 34 | 32 | 32 |
| | 6.4 | 5.4 | 11 | 14 | 22 | 24 | 32 | 33 | 32 | 37 |
| | 13 | 5.9 | 14 | 17 | 22 | 27 | 33 | 33 | 31 | 34 |
| | 26 | 4.5 | 8.9 | 16 | 28 | 28 | 31 | 34 | 36 | 40 |

TABLE 58

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Fexofenadine HCl (μM) | 0 | 15 | 15 | 18 | 27 | 33 | 39 | 40 | 45 | 45 |
| | 0.073 | 6.8 | 15 | 26 | 25 | 42 | 45 | 44 | 47 | 46 |
| | 0.15 | 8.4 | 17 | 24 | 32 | 39 | 39 | 46 | 46 | 44 |
| | 0.29 | 5.2 | 9.4 | 22 | 37 | 39 | 44 | 45 | 46 | 44 |
| | 0.58 | 11 | 16 | 22 | 38 | 40 | 38 | 46 | 50 | 43 |
| | 1.2 | 16 | 20 | 23 | 29 | 39 | 43 | 48 | 50 | 47 |
| | 2.3 | 8.4 | 21 | 22 | 35 | 39 | 41 | 45 | 50 | 49 |
| | 4.6 | 7.1 | 24 | 23 | 37 | 43 | 50 | 49 | 75 | 50 |
| | 9.3 | 14 | 23 | 24 | 38 | 44 | 45 | 46 | 45 | 50 |

TABLE 59

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Loratadine (μM) | 0 | 7 | 14 | 8.5 | 14 | 18 | 17 | 24 | 29 | 40 |
| | 0.2 | 5.7 | 14 | 16 | 15 | 18 | 25 | 33 | 33 | 39 |
| | 0.41 | 2.6 | 13 | 11 | 15 | 16 | 19 | 24 | 29 | 40 |
| | 0.82 | 5.6 | 12 | 9.4 | 11 | 24 | 23 | 26 | 31 | 39 |
| | 1.6 | 17 | 12 | 16 | 16 | 15 | 27 | 34 | 35 | 41 |
| | 3.3 | 21 | 20 | 26 | 26 | 25 | 32 | 40 | 47 | 45 |
| | 6.5 | 35 | 37 | 34 | 38 | 40 | 45 | 46 | 52 | 59 |
| | 13 | 55 | 54 | 55 | 57 | 55 | 65 | 63 | 63 | 70 |
| | 26 | 77 | 78 | 77 | 80 | 76 | 81 | 81 | 81 | 86 |

TABLE 60

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Meclizine HCl (μM) | 0 | −12 | −0.59 | 2.8 | 17 | 25 | 37 | 35 | 36 | 44 |
| | 0.32 | −4.9 | 7.4 | 15 | 11 | 24 | 32 | 35 | 40 | 44 |
| | 0.63 | −9.8 | −3.6 | 8.4 | 15 | 24 | 39 | 37 | 36 | 42 |
| | 1.3 | −5.6 | 0.99 | 8.7 | 24 | 31 | 29 | 31 | 36 | 39 |
| | 2.5 | −9.3 | 2 | 11 | 19 | 22 | 29 | 33 | 38 | 37 |
| | 5.1 | −0.7 | 1.4 | 13 | 15 | 20 | 33 | 34 | 41 | 34 |
| | 10 | 0.16 | 6.3 | 18 | 20 | 29 | 39 | 34 | 38 | 42 |
| | 20 | −1.9 | 13 | 18 | 26 | 37 | 42 | 44 | 46 | 45 |
| | 41 | 19 | 21 | 32 | 35 | 42 | 50 | 47 | 52 | 56 |

TABLE 61

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Promethazine HCl (μM) | 0 | 2.3 | −3.2 | 5.3 | 15 | 22 | 33 | 33 | 32 | 39 |
| | 0.24 | −3.5 | 3.2 | 8 | 25 | 23 | 30 | 35 | 34 | 38 |
| | 0.49 | −2.2 | −4.2 | −4.3 | 22 | 19 | 29 | 31 | 36 | 38 |
| | 0.97 | 2 | 6.2 | 11 | 14 | 28 | 26 | 30 | 29 | 32 |
| | 1.9 | 6.7 | 5 | 10 | 14 | 23 | 27 | 37 | 35 | 38 |
| | 3.9 | −3.8 | 18 | 19 | 20 | 21 | 36 | 35 | 36 | 40 |
| | 7.8 | 15 | 35 | 29 | 41 | 45 | 45 | 46 | 48 | 48 |
| | 16 | 42 | 46 | 55 | 54 | 59 | 67 | 65 | 65 | 62 |
| | 31 | 67 | 78 | 72 | 78 | 80 | 82 | 84 | 85 | 82 |

TABLE 62

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Pyrilamine Maleate (μM) | 0 | −6.1 | 5.1 | 5.4 | 11 | 18 | 24 | 25 | 24 | 31 |
| | 0.19 | −6.9 | 6.7 | 3 | 12 | 16 | 24 | 27 | 26 | 22 |
| | 0.38 | −6.8 | 0.6 | 3.9 | 10 | 16 | 20 | 23 | 29 | 11 |
| | 0.76 | −4.5 | 1.7 | 6.5 | 12 | 20 | 23 | 22 | 28 | 21 |
| | 1.5 | −6.3 | 3.3 | 6.4 | 14 | 17 | 24 | 28 | 35 | 31 |
| | 3 | 2.3 | 10 | 10 | 12 | 20 | 26 | 31 | 28 | 32 |
| | 6.1 | −0.24 | 5.8 | 11 | 13 | 20 | 30 | 31 | 34 | 34 |
| | 12 | 8.3 | 15 | 15 | 16 | 24 | 28 | 29 | 33 | 38 |
| | 24 | 11 | 15 | 16 | 29 | 32 | 34 | 33 | 40 | 42 |

TABLE 63

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Thiethylperazine Maleate | 0 | −1.3 | 8.3 | 11 | 12 | 26 | 34 | 36 | 39 | 39 |
| (μM) | 0.2 | −4.9 | 9.3 | 13 | 15 | 25 | 30 | 38 | 38 | 42 |
| | 0.39 | 0.79 | 10 | 16 | 17 | 25 | 31 | 38 | 37 | 37 |
| | 0.78 | 0.21 | 7 | 4.5 | 21 | 31 | 32 | 38 | 38 | 37 |
| | 1.6 | 5.3 | 13 | 9.5 | 19 | 26 | 25 | 32 | 34 | 36 |
| | 3.1 | 8.7 | 18 | 18 | 27 | 31 | 30 | 36 | 31 | 40 |
| | 6.3 | 21 | 29 | 31 | 34 | 39 | 41 | 47 | 43 | 53 |
| | 13 | 52 | 67 | 71 | 72 | 74 | 80 | 76 | 78 | 78 |
| | 25 | 93 | 94 | 91 | 89 | 90 | 95 | 91 | 88 | 94 |

TABLE 64

| | | Prednisolone (μM) | | | | | | | | |
|---|---|---|---|---|---|---|---|---|---|---|
| | | 0 | 0.0078 | 0.016 | 0.031 | 0.062 | 0.12 | 0.25 | 0.5 | 1 |
| Tripelennamine HCl (μM) | 0 | 5.1 | 6.6 | 9.8 | 12 | 27 | 37 | 35 | 35 | 40 |
| | 0.31 | 2.8 | 8.5 | 12 | 22 | 26 | 30 | 35 | 35 | 35 |
| | 0.61 | −0.97 | 6.8 | −1.5 | 14 | 27 | 31 | 37 | 36 | 27 |
| | 1.2 | 1.2 | −0.91 | 14 | 22 | 15 | 30 | 25 | 37 | 35 |
| | 2.4 | 8.7 | 1.3 | 16 | 21 | 24 | 25 | 34 | 36 | 33 |
| | 4.9 | −3.2 | 9.1 | 16 | 22 | 27 | 33 | 24 | 36 | 5.2 |
| | 9.8 | 2.5 | 6 | 9.1 | 26 | 30 | 23 | 38 | 36 | 38 |
| | 20 | 4 | 15 | 15 | 27 | 33 | 32 | 37 | 39 | 38 |
| | 39 | 21 | 19 | 22 | 28 | 36 | 44 | 43 | 44 | 45 |

TABLE 65

| | | Dipyridamole (μM) | | | | | | | | |
|---|---|---|---|---|---|---|---|---|---|---|
| | | 0 | 0.15 | 0.3 | 0.61 | 1.2 | 2.4 | 4.9 | 9.7 | 19 |
| Bromodiphenhydramine HCl | 0 | −4.6 | 3.4 | 0.92 | 6.6 | 12 | 25 | 40 | 54 | 71 |
| (μM) | 0.21 | 0 | 4.5 | 7.7 | 11 | 13 | 24 | 39 | 54 | 73 |
| | 0.43 | −4.4 | 7.6 | 5.1 | 11 | 15 | 23 | 41 | 53 | 71 |
| | 0.85 | 0.72 | 9 | 8.6 | 15 | 23 | 25 | 40 | 55 | 71 |
| | 1.7 | −2.4 | 5.1 | 7.4 | 10 | 20 | 31 | 41 | 54 | 67 |
| | 3.4 | 5.6 | 17 | 19 | 22 | 30 | 36 | 45 | 58 | 74 |
| | 6.8 | 13 | 24 | 25 | 26 | 46 | 47 | 56 | 70 | 75 |
| | 14 | 34 | 45 | 41 | 43 | 47 | 60 | 65 | 67 | 77 |
| | 27 | 61 | 61 | 66 | 72 | 70 | 70 | 77 | 76 | 82 |

TABLE 66

| | | Dipyridamole (μM) | | | | | | | | |
|---|---|---|---|---|---|---|---|---|---|---|
| | | 0 | 0.15 | 0.3 | 0.61 | 1.2 | 2.4 | 4.9 | 9.7 | 19 |
| Cyproheptadine HCl (μM) | 0 | −4.2 | 4.5 | −0.27 | 4.3 | 0.57 | 12 | 33 | 49 | 63 |
| | 0.24 | 0.32 | −1.3 | −1.3 | 5.7 | 4.6 | 15 | 38 | 50 | 71 |
| | 0.48 | 2 | 12 | 4.6 | 7 | 13 | 19 | 34 | 52 | 64 |
| | 0.96 | 1.2 | 12 | 5.1 | 9.9 | 8.7 | 21 | 41 | 54 | 68 |
| | 1.9 | 4.6 | 9.2 | 10 | 15 | 21 | 25 | 43 | 55 | 65 |
| | 3.9 | 2.4 | 13 | 21 | 22 | 23 | 32 | 47 | 62 | 71 |
| | 7.7 | 15 | 32 | 36 | 45 | 40 | 47 | 61 | 66 | 78 |
| | 15 | 29 | 44 | 46 | 50 | 60 | 56 | 70 | 68 | 84 |
| | 31 | 29 | 44 | 46 | 50 | 60 | 73 | 70 | 80 | 84 |

TABLE 67

| | | Dipyridamole (μM) | | | | | | | | |
|---|---|---|---|---|---|---|---|---|---|---|
| | | 0 | 0.15 | 0.3 | 0.61 | 1.2 | 2.4 | 4.9 | 9.7 | 19 |
| Loratadine | 0 | −6.4 | 6.5 | 9.6 | 12 | 16 | 30 | 37 | 55 | 70 |
| (μM) | 0.2 | 0 | 7.5 | 6.4 | 11 | 14 | 28 | 40 | 52 | 71 |
| | 0.41 | −6.2 | 3.4 | 5.5 | 14 | 13 | 29 | 41 | 62 | 70 |
| | 0.82 | −2.1 | 4.8 | 3.9 | 5.3 | 11 | 23 | 39 | 54 | 71 |
| | 1.6 | −2.4 | 7.4 | 6 | 12 | 13 | 26 | 45 | 58 | 71 |
| | 3.3 | 8.1 | 17 | 12 | 20 | 24 | 35 | 45 | 61 | 73 |
| | 6.5 | 11 | 21 | 25 | 31 | 34 | 44 | 65 | 68 | 80 |
| | 13 | 31 | 44 | 42 | 47 | 49 | 61 | 67 | 77 | 67 |
| | 26 | 31 | 44 | 42 | 47 | 49 | 61 | 67 | 77 | 66 |

TABLE 68

| | | Dipyridamole (μM) | | | | | | | | |
|---|---|---|---|---|---|---|---|---|---|---|
| | | 0 | 0.15 | 0.3 | 0.61 | 1.2 | 2.4 | 4.9 | 9.7 | 19 |
| Thiethylperazine Maleate (μM) | 0 | −4.1 | 6.1 | 7 | 9.1 | 19 | 27 | 40 | 54 | 71 |
| | 0.2 | −3.3 | 6.4 | 12 | 11 | 18 | 26 | 42 | 54 | 72 |
| | 0.39 | −3.9 | 8.2 | 11 | 16 | 18 | 26 | 44 | 55 | 71 |
| | 0.78 | −1 | 3.8 | 9.8 | 16 | 20 | 29 | 42 | 56 | 70 |
| | 1.6 | −3.4 | 5.1 | 4.6 | 16 | 18 | 28 | 42 | 56 | 71 |
| | 3.1 | 1.4 | 15 | 16 | 20 | 24 | 37 | 49 | 62 | 70 |
| | 6.3 | 15 | 27 | 27 | 34 | 41 | 50 | 61 | 71 | 82 |
| | 13 | 51 | 62 | 61 | 64 | 67 | 72 | 79 | 85 | 89 |
| | 25 | 62 | 75 | 76 | 77 | 88 | 87 | 87 | 90 | 95 |

TABLE 69

| | | Ibudilast (μM) | | | | | | | | |
|---|---|---|---|---|---|---|---|---|---|---|
| | | 0 | 0.062 | 0.12 | 0.25 | 0.5 | 1 | 2 | 4 | 8 |
| Bromodiphenhydramine HCl | 0 | −3.8 | 5.9 | 2 | 15 | 19 | 24 | 36 | 43 | 41 |
| (μM) | 0.21 | −1.7 | 6.1 | 9.7 | 13 | 23 | 26 | 35 | 40 | 41 |
| | 0.43 | −3.3 | 6.1 | 5.6 | 15 | 21 | 25 | 35 | 40 | 38 |
| | 0.85 | 0.96 | 4.7 | 6.6 | 12 | 19 | 29 | 35 | 41 | 44 |
| | 1.7 | −3.8 | 7.1 | 4.5 | 6.7 | 15 | 27 | 33 | 37 | 41 |
| | 3.4 | 5 | 12 | 9.3 | 15 | 19 | 32 | 36 | 42 | 45 |
| | 6.8 | 11 | 19 | 21 | 22 | 32 | 34 | 44 | 45 | 50 |
| | 14 | 33 | 40 | 39 | 44 | 41 | 46 | 45 | 42 | 53 |
| | 27 | 65 | 64 | 65 | 70 | 72 | 73 | 75 | 74 | 77 |

TABLE 70

| | | Ibudilast (μM) | | | | | | | | |
|---|---|---|---|---|---|---|---|---|---|---|
| | 9 × 9 | 0 | 0.062 | 0.12 | 0.25 | 0.5 | 1 | 2 | 4 | 8 |
| Cyproheptadine HCl (μM) | 0 | −2.6 | 2.2 | 8.3 | 16 | 20 | 23 | 32 | 36 | 41 |
| | 0.24 | 1.4 | 6 | 4.5 | 17 | 17 | 26 | 26 | 32 | 39 |
| | 0.48 | 0.95 | 6 | 7.5 | 13 | 17 | 25 | 27 | 37 | 39 |
| | 0.96 | 1.9 | 9 | 11 | 8.7 | 16 | 30 | 31 | 38 | 38 |
| | 1.9 | 1.6 | 9.5 | 7 | 13 | 17 | 26 | 28 | 33 | 39 |
| | 3.9 | 13 | 22 | 17 | 21 | 21 | 36 | 34 | 40 | 46 |
| | 7.7 | 27 | 29 | 30 | 34 | 35 | 45 | 47 | 53 | 53 |
| | 15 | 49 | 55 | 55 | 59 | 58 | 63 | 63 | 68 | 69 |

TABLE 71

| | | Ibudilast (μM) | | | | | | | | |
|---|---|---|---|---|---|---|---|---|---|---|
| | | 0 | 0.062 | 0.12 | 0.25 | 0.5 | 1 | 2 | 4 | 8 |
| Loratadine | 0 | −0.97 | 7.6 | 6.8 | 17 | 21 | 32 | 35 | 39 | 43 |
| (μM) | 0.2 | −0.39 | 7.6 | 8.2 | 17 | 21 | 32 | 34 | 38 | 42 |
| | 0.41 | −7.7 | 0.67 | 6.6 | 9.4 | 20 | 26 | 35 | 35 | 39 |
| | 0.82 | 0.1 | 5.7 | 8.4 | 14 | 19 | 27 | 37 | 40 | 39 |
| | 1.6 | −1.5 | 3.9 | 1.3 | 12 | 19 | 30 | 33 | 38 | 39 |
| | 3.3 | 8.4 | 10 | 11 | 18 | 25 | 31 | 38 | 42 | 47 |
| | 6.5 | 14 | 22 | 24 | 26 | 32 | 35 | 43 | 48 | 50 |
| | 13 | 28 | 38 | 32 | 40 | 44 | 50 | 51 | 59 | 59 |
| | 26 | 28 | 38 | 39 | 40 | 44 | 50 | 51 | 59 | 64 |

TABLE 72

| | | Ibudilast (μM) | | | | | | | | |
|---|---|---|---|---|---|---|---|---|---|---|
| | | 0 | 0.062 | 0.12 | 0.25 | 0.5 | 1 | 2 | 4 | 8 |
| Thiethylperazine Maleate (μM) | 0 | −2 | 7.1 | 8.4 | 15 | 23 | 27 | 38 | 42 | 48 |
| | 0.2 | −1.8 | 0.66 | 7.4 | 16 | 22 | 30 | 36 | 40 | 45 |
| | 0.39 | −5.9 | −0.82 | 8.8 | 11 | 21 | 25 | 34 | 37 | 43 |
| | 0.78 | −4.4 | 0.75 | 10 | 11 | 18 | 24 | 31 | 37 | 40 |
| | 1.6 | −2.6 | −0.63 | 6 | 15 | 18 | 24 | 31 | 36 | 38 |
| | 3.1 | −2.9 | 10 | 8.5 | 14 | 21 | 27 | 34 | 36 | 38 |
| | 6.3 | 11 | 16 | 23 | 22 | 32 | 33 | 38 | 46 | 49 |
| | 13 | 43 | 49 | 48 | 53 | 54 | 61 | 64 | 65 | 68 |
| | 25 | 61 | 77 | 80 | 81 | 82 | 82 | 79 | 84 | 85 |

TABLE 73

| | | Rolipram (μM) | | | | | | | | |
|---|---|---|---|---|---|---|---|---|---|---|
| | | 0 | 0.0079 | 0.016 | 0.031 | 0.063 | 0.13 | 0.25 | 0.5 | 1 |
| Bromodiphenhydramine | 0 | −0.64 | 9.2 | 6.5 | 11 | 15 | 26 | 29 | 37 | 44 |
| HCl (μM) | 0.21 | −4.1 | 1.2 | 12 | 9.8 | 18 | 26 | 32 | 35 | 47 |
| | 0.43 | −4.5 | 6.2 | 5 | 10 | 19 | 21 | 28 | 39 | 41 |
| | 0.85 | 2.7 | 7.5 | 1.8 | 13 | 15 | 26 | 31 | 40 | 44 |
| | 1.7 | 2.8 | 6.3 | 6.5 | 10 | 19 | 24 | 30 | 39 | 43 |
| | 3.4 | 0.16 | 10 | 13 | 13 | 20 | 25 | 34 | 40 | 45 |
| | 6.8 | 14 | 22 | 28 | 27 | 37 | 39 | 40 | 51 | 53 |
| | 14 | 34 | 41 | 47 | 46 | 45 | 51 | 54 | 61 | 67 |
| | 27 | 33 | 41 | 47 | 58 | 54 | 62 | 71 | 70 | 82 |

TABLE 74

| | | Rolipram (μM) | | | | | | | | |
|---|---|---|---|---|---|---|---|---|---|---|
| | | 0 | 0.0079 | 0.016 | 0.031 | 0.063 | 0.13 | 0.25 | 0.5 | 1 |
| Cyproheptadine HCl (μM) | 0 | | 11 | 15 | 19 | 23 | 29 | 37 | 44 | 38 |
| | 0.24 | −0.13 | 15 | 21 | 16 | 24 | 33 | 35 | 42 | 46 |
| | 0.48 | 0.022 | 9.4 | 15 | 20 | 27 | 34 | 37 | 45 | 50 |
| | 0.96 | 6.9 | 13 | 24 | 21 | 33 | 42 | 41 | 44 | 48 |
| | 1.9 | 3 | 16 | 16 | 16 | 25 | 29 | 40 | 37 | 39 |
| | 3.9 | 15 | 17 | 18 | 24 | 31 | 32 | 41 | 43 | 48 |
| | 7.7 | 24 | 33 | 32 | 29 | 36 | 45 | 45 | 53 | 53 |
| | 15 | 46 | 53 | 56 | 53 | 57 | 58 | 60 | 66 | 66 |
| | 31 | 53 | 75 | 75 | 76 | 76 | 78 | 77 | 77 | 79 |

TABLE 75

| | | Rolipram (μM) | | | | | | | | |
|---|---|---|---|---|---|---|---|---|---|---|
| | | 0 | 0.0079 | 0.016 | 0.031 | 0.063 | 0.13 | 0.25 | 0.5 | 1 |
| Loratadine (μM) | 0 | −0.79 | 9.9 | 13 | 15 | 17 | 27 | 32 | 43 | 50 |
| | 0.2 | −0.36 | 9.2 | 13 | 14 | 22 | 24 | 31 | 44 | 47 |
| | 0.41 | 1.9 | 8.2 | 13 | 17 | 21 | 28 | 31 | 40 | 45 |
| | 0.82 | 3.3 | 11 | 13 | 17 | 20 | 29 | 34 | 41 | 46 |
| | 1.6 | 2.8 | 13 | 15 | 19 | 26 | 33 | 32 | 42 | 47 |
| | 3.3 | 10 | 19 | 22 | 25 | 31 | 39 | 39 | 45 | 52 |
| | 6.5 | 25 | 26 | 31 | 35 | 39 | 43 | 48 | 55 | 58 |
| | 13 | 43 | 46 | 48 | 51 | 45 | 57 | 61 | 65 | 64 |
| | 26 | 43 | 46 | 57 | 58 | 64 | 57 | 61 | 65 | 74 |

TABLE 76

| | | Rolipram (μM) | | | | | | | | |
|---|---|---|---|---|---|---|---|---|---|---|
| | | 0 | 0.0079 | 0.016 | 0.031 | 0.063 | 0.13 | 0.25 | 0.5 | 1 |
| Thiethylperazine Maleate | 0 | −0.55 | 9 | 14 | 19 | 20 | 28 | 30 | 38 | 39 |
| (μM) | 0.2 | −0.086 | 6 | 14 | 16 | 24 | 30 | 37 | 35 | 42 |
| | 0.39 | −7.4 | 7.5 | 6.2 | 14 | 27 | 32 | 35 | 42 | 45 |
| | 0.78 | −4.6 | 2.3 | 14 | 16 | 20 | 34 | 34 | 42 | 40 |
| | 1.6 | −7.8 | 2.7 | 7.3 | 8.1 | 20 | 25 | 33 | 34 | 33 |
| | 3.1 | −5.8 | 4.3 | 5.4 | 11 | 16 | 27 | 28 | 34 | 35 |
| | 6.3 | 4 | 10 | 12 | 20 | 22 | 25 | 34 | 38 | 36 |
| | 13 | 30 | 34 | 36 | 41 | 42 | 47 | 50 | 52 | 55 |
| | 25 | 30 | 34 | 36 | 41 | 46 | 51 | 52 | 47 | 55 |

TABLE 77

| | | Cyclosporine (μM) | | | | | | | | |
|---|---|---|---|---|---|---|---|---|---|---|
| | | 0 | 0.0019 | 0.0039 | 0.0077 | 0.015 | 0.031 | 0.062 | 0.12 | 0.25 |
| Desloratadine (μM) | 0 | −0.178 | 1.953 | 0.975 | 6.922 | 17.44 | 33.95 | 55.98 | 72.58 | 90.68 |
| | 0.25 | −1.255 | 5.065 | 3.345 | 10.4 | 21.28 | 36.2 | 55.17 | 75.58 | 91.6 |
| | 0.51 | −4.652 | 3.805 | 5.8 | 5.505 | 14.89 | 32.55 | 58.65 | 79.03 | 92 |
| | 1 | 6.598 | 7.185 | 7.982 | 12.26 | 21.1 | 38.65 | 65.02 | 82.45 | 92.93 |
| | 2 | 10.61 | 15.79 | 19.43 | 25.43 | 32.85 | 51.05 | 66.6 | 84.27 | 92.53 |
| | 4.1 | 31.45 | 38.38 | 33 | 38.95 | 48.93 | 64.78 | 78.58 | 90.38 | 93.78 |
| | 8.1 | 56 | 58.73 | 60.02 | 63 | 71.58 | 78.9 | 87.2 | 93.77 | 95.15 |
| | 16 | 82.18 | 84.38 | 83.05 | 85.28 | 89.5 | 91.95 | 94.2 | 96 | 95.83 |
| | 33 | 89.4 | 95.05 | 94.75 | 94.97 | 96.07 | 95.45 | 94.42 | 96.8 | 95.62 |

TABLE 78

| | | Cyclosporine (μM) | | | | | | | | |
|---|---|---|---|---|---|---|---|---|---|---|
| | | 0 | 0.0019 | 0.0039 | 0.0077 | 0.015 | 0.031 | 0.062 | 0.12 | 0.25 |
| Loratadine (μM) | 0 | −0.373 | 1.825 | 5.875 | 11.71 | 25.85 | 52.45 | 75.95 | 89 | 91.95 |
| | 0.2 | 0 | 1.041 | 4.4 | 13.2 | 29.1 | 52.4 | 78.75 | 90.35 | 92.95 |
| | 0.41 | −2.384 | 2.075 | 3.525 | 11.39 | 27.15 | 49.7 | 79.05 | 90.55 | 91.15 |
| | 0.82 | 0.362 | 0.16 | 8.96 | 13.9 | 31.4 | 53.5 | 81.75 | 91.3 | 91.65 |
| | 1.6 | 3.4 | 5.35 | 13.2 | 19.4 | 36.3 | 61.85 | 83.45 | 91.35 | 90.55 |
| | 3.3 | 4.83 | 14.5 | 5.785 | 24.7 | 38.2 | 63.5 | 84.5 | 89.25 | 91.15 |
| | 6.5 | 19.45 | 27.3 | 22.2 | 37.1 | 50.85 | 70.4 | 84.35 | 90.15 | 91 |
| | 13 | 30.1 | 36.95 | 36.15 | 46 | 61.45 | 73.9 | 88.1 | 91.65 | 92.7 |
| | 26 | 40.7 | 51.25 | 50.9 | 55.35 | 65.6 | 74.4 | 89.3 | 92.05 | 92.15 |

TABLE 79

| | | Prednisolone (μM) | | | | | | | |
|---|---|---|---|---|---|---|---|---|---|
| | | 0 | 0.27 | 0.54 | 1.1 | 2.2 | 4.3 | 8.7 | 17 |
| Epinastine HCl (μM) | 0 | −4.97 | 4.4 | 5.21 | 15.1 | 14.4 | 12.7 | 23 | 33.4 |
| | 0.0077 | 2.14 | 6.29 | 12.5 | 15.7 | 14.6 | 13.6 | 22.6 | 35.8 |
| | 0.015 | 5.96 | 12.4 | 14.4 | 13.8 | 15.6 | 16.9 | 29.4 | 36.8 |
| | 0.031 | 18.7 | 18.6 | 16.6 | 21.8 | 21.8 | 27.2 | 34.9 | 44.1 |
| | 0.062 | 30.2 | 32.2 | 27.6 | 31.1 | 30.3 | 34.2 | 41.8 | 51.8 |
| | 0.12 | 32.6 | 37.4 | 34.9 | 37 | 37.4 | 42.3 | 49.4 | 53 |
| | 0.25 | 35.2 | 39.5 | 41.1 | 42 | 43.2 | 43.7 | 48.8 | 55.9 |
| | 0.49 | 38.6 | 40.1 | 41.9 | 44 | 41.2 | 46.6 | 50 | 60.1 |
| | 0.99 | 42.4 | 41.7 | 45.1 | 45.3 | 46.3 | 47.4 | 53.2 | 59.2 |

TABLE 80

| | | Fluoxetine HCl (μM) | | | | | | | | |
|---|---|---|---|---|---|---|---|---|---|---|
| | | 0 | 0.23 | 0.45 | 0.9 | 1.8 | 3.6 | 7.2 | 14 | 29 |
| Bromodiphenhydramine | 0 | −13 | −9.9 | −15 | −14 | −12 | −17 | −11 | 0.4 | 35 |
| HCl (μM) | 0.21 | −16 | −12 | −15 | −15 | −12 | −16 | −7.8 | −0.49 | 39 |
| | 0.43 | −15 | −13 | −18 | −15 | −7.8 | −16 | −14 | −3.7 | 37 |
| | 0.85 | −14 | −11 | −18 | −16 | −12 | −18 | −7.8 | −1.4 | 39 |
| | 1.7 | −15 | −13 | −13 | −16 | −11 | −14 | −9 | 5.5 | 42 |
| | 3.4 | −12 | −7.7 | −13 | −9.4 | −8.7 | −8.9 | −2.7 | 16 | 52 |
| | 6.8 | −2.5 | 2.1 | −4.2 | −1.6 | 5.5 | 6.2 | 18 | 32 | 60 |
| | 14 | 30 | 37 | 33 | 34 | 43 | 43 | 52 | 62 | 77 |
| | 27 | 73 | 73 | 74 | 74 | 81 | 80 | 81 | 82 | 86 |

TABLE 81

| | | Fluoxetine HCl (μM) | | | | | | | | |
|---|---|---|---|---|---|---|---|---|---|---|
| | | 0 | 0.23 | 0.45 | 0.9 | 1.8 | 3.6 | 7.2 | 14 | 29 |
| Cyproheptadine HCl (μM) | 0 | −4.4 | −13 | −12 | −6.2 | −6.9 | −7 | −3.7 | 3 | 32 |
| | 0.24 | −4.3 | −6.2 | −7 | −2.9 | −6.6 | −14 | −3.2 | 6.2 | 32 |
| | 0.48 | −5.6 | −7.7 | −8.5 | −4.3 | −7.7 | −12 | −2.2 | 7.5 | 35 |
| | 0.96 | −2 | −6.7 | −9.1 | 0.27 | −3.8 | −5.7 | −2.5 | 12 | 42 |
| | 1.9 | 0.005 | −4.8 | −7 | −4.9 | −6.7 | −2.6 | 8 | 16 | 37 |
| | 3.9 | 1.7 | 2.7 | 1.1 | 4.7 | 2.9 | 4.9 | 15 | 28 | 55 |
| | 7.7 | 26 | 24 | 23 | 25 | 29 | 39 | 45 | 49 | 67 |
| | 15 | 59 | 66 | 61 | 63 | 67 | 68 | 71 | 75 | 83 |
| | 31 | 86 | 86 | 87 | 90 | 90 | 89 | 91 | 91 | 93 |

TABLE 82

| | | Fluoxetine HCl (μM) | | | | | | | | |
|---|---|---|---|---|---|---|---|---|---|---|
| | | 0 | 0.23 | 0.45 | 0.9 | 1.8 | 3.6 | 7.2 | 14 | 29 |
| Loratadine (μM) | 0 | −14 | −20 | −20 | −15 | −10 | −15 | −9.1 | 5.7 | 38 |
| | 0.2 | −10 | −15 | −20 | −18 | −18 | −16 | −11 | 6.6 | 49 |
| | 0.41 | −11 | −13 | −20 | −20 | −20 | −16 | −8.5 | 10 | 53 |
| | 0.82 | −12 | −20 | −20 | −20 | −20 | −17 | −8.1 | 11 | 58 |
| | 1.6 | −8.1 | −20 | −15 | −19 | −20 | −8.9 | 10 | 37 | 59 |
| | 3.3 | −12 | −7.6 | −16 | −18 | 0.035 | 16 | 30 | 49 | 65 |
| | 6.5 | 1.6 | −6.8 | 1.1 | −0.98 | 8.8 | 27 | 41 | 53 | 64 |
| | 13 | 16 | 11 | 12 | 20 | 24 | 32 | 50 | 64 | 72 |
| | 26 | 19 | 20 | 31 | 31 | 39 | 38 | 53 | 64 | 76 |

TABLE 83

| | | Fluoxetine HCl (μM) | | | | | | | | |
|---|---|---|---|---|---|---|---|---|---|---|
| | | 0 | 0.23 | 0.45 | 0.9 | 1.8 | 3.6 | 7.2 | 14 | 29 |
| Thiethylperazine Maleate | 0 | −13 | −8.1 | −8.3 | −14 | −14 | −11 | −15 | −6.2 | 29 |
| (μM) | 0.2 | −12 | −19 | −20 | −19 | −17 | −16 | −11 | −13 | 7.2 |
| | 0.39 | −18 | −18 | −20 | −18 | −18 | −16 | −15 | −8.6 | 20 |
| | 0.78 | −14 | −16 | −20 | −20 | −20 | −19 | −15 | −16 | 18 |
| | 1.6 | −18 | −17 | −16 | −19 | −20 | −17 | −13 | −9.1 | 19 |
| | 3.1 | −18 | −20 | −20 | −20 | −20 | −20 | −20 | −10 | 15 |
| | 6.3 | −15 | −14 | −15 | −17 | −12 | −13 | 0.17 | 4.7 | 37 |
| | 13 | 36 | 44 | 45 | 42 | 48 | 50 | 49 | 63 | 79 |
| | 25 | 90 | 90 | 90 | 90 | 91 | 92 | 93 | 93 | 94 |

TABLE 84

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Bromodiphenhydramine | 0 | −20 | −16 | −20 | −15 | −18 | 0.95 | 33 | 65 | 96 |
| HCl (μM) | 0.21 | −20 | −16 | −20 | −20 | −14 | −16 | 29 | 67 | 38 |
| | 0.43 | −20 | −20 | −20 | −20 | −20 | −15 | 23 | 66 | 96 |
| | 0.85 | −20 | −20 | −20 | −20 | −20 | −15 | 27 | 65 | 97 |
| | 1.7 | −20 | −20 | −20 | −20 | −20 | −7.3 | 28 | 69 | 96 |
| | 3.4 | −20 | −20 | −20 | −20 | −20 | −4 | 36 | 72 | 95 |
| | 6.8 | −20 | −19 | −20 | −19 | −5.6 | 20 | 53 | 79 | 97 |
| | 14 | 5.9 | 17 | 17 | 20 | 32 | 56 | 72 | 91 | 97 |
| | 27 | 64 | 66 | 64 | 71 | 72 | 80 | 89 | 93 | 89 |

TABLE 85

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Cyproheptadine HCl (μM) | 0 | −16 | −16 | −18 | −13 | −18 | 0.035 | 27 | 62 | 91 |
| | 0.24 | −16 | −20 | −20 | −17 | −15 | −3.6 | 29 | 69 | 90 |
| | 0.48 | −20 | −20 | −20 | −20 | −19 | −5.6 | 28 | 66 | 93 |
| | 0.96 | −20 | −20 | −20 | −20 | −20 | −7.6 | 33 | 70 | 89 |
| | 1.9 | −20 | −20 | −19 | −20 | −18 | −0.25 | 32 | 69 | 90 |
| | 3.9 | −18 | −20 | −18 | −19 | −9.9 | 8.9 | 40 | 73 | 91 |
| | 7.7 | −8 | −13 | −10 | −3.8 | 14 | 36 | 58 | 79 | 92 |
| | 15 | 31 | 37 | 34 | 39 | 49 | 60 | 73 | 88 | 93 |
| | 31 | 73 | 73 | 73 | 75 | 80 | 83 | 87 | 90 | 91 |

TABLE 86

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Loratadine (μM) | 0 | 6.24 | −12.4 | 4.81 | 4.81 | 10.5 | 22.5 | 51.1 | 82.8 | 97.4 |
| | 0.2 | 0 | −4.51 | −1.67 | 3.7 | −1.57 | 39 | 55.1 | 87 | 98 |
| | 0.41 | −20 | 9.02 | 0.68 | 1.73 | 26.3 | 44.2 | 65.7 | 84.7 | 97.5 |
| | 0.82 | −0.115 | −6.23 | −1.08 | 12.1 | 15 | 47.4 | 65.2 | 82.8 | 97.8 |
| | 1.6 | −5.24 | −19.5 | 3.69 | 20.6 | 33.6 | 58.2 | 72.8 | 87 | 97.9 |
| | 3.3 | 0.437 | 21.8 | 18.8 | 33.6 | 44.1 | 61.5 | 74.4 | 89.3 | 96.8 |
| | 6.5 | 5.84 | 17.4 | 21.1 | 35.7 | 59.8 | 66.4 | 79.7 | 93.8 | 98.1 |
| | 13 | 19.5 | 29.4 | 37.2 | 54.8 | 63.5 | 75.4 | 84.6 | 95.7 | 97.5 |
| | 26 | 30.6 | 49.7 | 52.7 | 63.9 | 75.5 | 81.9 | 91.8 | 96.7 | 98.3 |

TABLE 87

| | | Paroxetine HCl (μM) | | | | | | | | |
|---|---|---|---|---|---|---|---|---|---|---|
| | | 0 | 0.21 | 0.42 | 0.83 | 1.7 | 3.3 | 6.7 | 13 | 27 |
| Thiethylperazine Maleate | 0 | −12 | −18 | −17 | −13 | −12 | 3.7 | 43 | 73 | 95 |
| (μM) | 0.2 | −8.7 | −14 | −19 | −13 | −15 | 0.59 | 42 | 62 | 72 |
| | 0.39 | −15 | −16 | −20 | −15 | −12 | −2.1 | 39 | 71 | 95 |
| | 0.78 | −18 | −16 | −20 | −18 | −15 | 2.9 | 38 | 76 | 91 |
| | 1.6 | −12 | −17 | −16 | −19 | −14 | 7 | 46 | 79 | 96 |
| | 3.1 | −14 | −15 | −20 | −17 | −11 | 17 | 49 | 80 | 96 |
| | 6.3 | −3.2 | −5.6 | −9.9 | 3.2 | 14 | 39 | 67 | 89 | 96 |
| | 13 | 45 | 49 | 56 | 55 | 68 | 76 | 89 | 96 | 94 |
| | 25 | 91 | 92 | 92 | 94 | 95 | 94 | 96 | 97 | 97 |

TABLE 88

| | | Amoxapine(μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Bromodiphenhydramine | 0 | −10 | −12 | −5.6 | −1.4 | −5.8 | 15 | 39 | 66 | 84 |
| HCl (μM) | 0.21 | −3.9 | −19 | −16 | −5.8 | −5 | 8.4 | 33 | 66 | 62 |
| | 0.43 | −17 | −17 | −16 | −14 | −4.2 | 6 | 34 | 67 | 87 |
| | 0.85 | −17 | −19 | −19 | −16 | −8.3 | −0.69 | 34 | 66 | 87 |
| | 1.7 | −20 | −17 | −17 | −17 | −10 | 11 | 40 | 69 | 86 |
| | 3.4 | −18 | −19 | −18 | −14 | −4.5 | 17 | 47 | 72 | 89 |
| | 6.8 | −17 | −10 | −8.2 | −0.42 | 17 | 35 | 63 | 79 | 90 |
| | 14 | 17 | 28 | 28 | 37 | 52 | 66 | 77 | 88 | 90 |
| | 27 | 60 | 71 | 70 | 74 | 80 | 83 | 89 | 92 | 93 |

TABLE 89

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Cyproheptadine HCl (μM) | 0 | −19 | −18 | −17 | −13 | −8.1 | 5.1 | 32 | 68 | 92 |
| | 0.24 | −14 | −16 | −20 | −16 | −7.7 | 7.3 | 34 | 67 | 64 |
| | 0.48 | −18 | −17 | −18 | −12 | −4.3 | 0.83 | 28 | 66 | 90 |
| | 0.96 | −17 | −15 | −15 | −14 | −6.4 | 1.5 | 34 | 68 | 91 |
| | 1.9 | −17 | −16 | −13 | −11 | −6.2 | 5 | 45 | 73 | 90 |
| | 3.9 | −14 | −11 | −14 | −9.6 | −4.7 | 19 | 52 | 78 | 92 |
| | 7.7 | −1.5 | −1.9 | 2.3 | 7.8 | 28 | 48 | 72 | 86 | 95 |
| | 15 | 32 | 47 | 44 | 50 | 61 | 77 | 88 | 95 | 97 |
| | 31 | 79 | 82 | 83 | 89 | 90 | 94 | 97 | 97 | 97 |

TABLE 90

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Loratadine (μM) | 0 | 1.82 | 0.598 | 4.39 | 8.02 | 16.4 | 30 | 51.1 | 77.4 | 85.7 |
| | 0.2 | 0.963 | 4.67 | 5.84 | 14 | 17 | 32.8 | 56.6 | 80.7 | 92.1 |
| | 0.41 | 5.88 | 3.08 | 4.96 | 13 | 23.7 | 34.2 | 60.3 | 82 | 93 |
| | 0.82 | 2.55 | 5.45 | 8.6 | 14.6 | 25.5 | 40.1 | 66 | 84 | 93.5 |
| | 1.6 | 6.14 | 5.48 | 7.75 | 18.9 | 34.7 | 48 | 69.9 | 85.8 | 88.5 |
| | 3.3 | 3.56 | 12.7 | 15.3 | 25 | 40.4 | 55.1 | 74.6 | 85.5 | 92 |
| | 6.5 | 13.8 | 25.1 | 30.4 | 41.6 | 53.8 | 65.7 | 79.7 | 87.5 | 93.4 |
| | 13 | 22.6 | 42.5 | 38.7 | 50.7 | 61.7 | 72.8 | 82 | 87.1 | 91 |
| | 26 | 40.2 | 56.7 | 57.9 | 64.9 | 75.7 | 80.7 | 87.1 | 93.5 | 92.6 |

TABLE 91

| | | Amoxapine (μM) | | | | | | | | |
|---|---|---|---|---|---|---|---|---|---|---|
| | | 0 | 0.2 | 0.4 | 0.8 | 1.6 | 3.2 | 6.4 | 13 | 25 |
| Thiethylperazine Maleate | 0 | −9.6 | −16 | −9.8 | −6.4 | 4.6 | 18 | 48 | 77 | 95 |
| (μM) | 0.2 | −9.9 | −11 | −14 | −1.5 | 1.7 | 12 | 43 | 77 | 95 |
| | 0.39 | −16 | −13 | −13 | −7.4 | 1.8 | 10 | 43 | 75 | 94 |
| | 0.78 | −13 | −13 | −16 | −12 | −0.91 | 10 | 38 | 75 | 95 |
| | 1.6 | −12 | −11 | −15 | −9.7 | 0.5 | 15 | 46 | 75 | 90 |
| | 3.1 | −12 | −7.3 | −16 | −11 | 4.8 | 22 | 58 | 84 | 96 |
| | 6.3 | −0.65 | 4.6 | 8.5 | 20 | 34 | 54 | 81 | 92 | 97 |
| | 13 | 47 | 59 | 58 | 72 | 80 | 89 | 95 | 98 | 98 |
| | 25 | 91 | 94 | 95 | 96 | 97 | 97 | 98 | 98 | 98 |

TABLE 92

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Bromodiphenydramine | 0 | −4.9 | −8.2 | −9.3 | −6.7 | −3 | 0.15 | 31 | 69 | 94 |
| HCl (μM) | 0.21 | −7.5 | −14 | −12 | −12 | −9.2 | −3.7 | 30 | 71 | 93 |
| | 0.43 | −12 | −16 | −14 | −10 | −8.9 | −1.3 | 32 | 70 | 95 |
| | 0.85 | −12 | −16 | −18 | −10 | −10 | 0.4 | 34 | 70 | 95 |
| | 1.7 | −14 | −9.4 | −12 | −9.4 | −6.2 | 8.2 | 43 | 77 | 95 |
| | 3.4 | −11 | −5.8 | −11 | −6.9 | 4.3 | 26 | 55 | 80 | 96 |
| | 6.8 | 4.4 | 5.7 | 9.8 | 11 | 21 | 44 | 62 | 83 | 96 |
| | 14 | 35 | 42 | 40 | 48 | 59 | 72 | 79 | 91 | 96 |
| | 27 | 77 | 78 | 78 | 82 | 84 | 88 | 92 | 95 | 97 |

TABLE 93

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Cyproheptadine HCl (μM) | 0 | −11 | −19 | −16 | −17 | −9.1 | −14 | 18 | 60 | 94 |
| | 0.24 | −15 | −17 | −20 | −16 | −17 | −14 | 22 | 69 | 95 |
| | 0.48 | −7.6 | −20 | −20 | −19 | −18 | −12 | 16 | 56 | 92 |
| | 0.96 | −16 | −20 | −20 | −19 | −17 | −16 | 22 | 57 | 95 |
| | 1.9 | −13 | −18 | −20 | −16 | −16 | −8.2 | 29 | 71 | 95 |
| | 3.9 | −12 | −20 | −20 | −17 | −12 | 10 | 48 | 76 | 96 |
| | 7.7 | 1.2 | −1.6 | −2.8 | 7.9 | 13 | 35 | 57 | 78 | 95 |
| | 15 | 27 | 43 | 33 | 43 | 46 | 62 | 79 | 91 | 94 |
| | 31 | 74 | 74 | 75 | 78 | 81 | 85 | 91 | 95 | 94 |

TABLE 94

| | | | Nortriptyline HCl (μM) | | | | | | | |
|---|---|---|---|---|---|---|---|---|---|---|
| | | 0 | 0.26 | 0.52 | 1 | 2.1 | 4.2 | 8.3 | 17 | 33 |
| Loratadine (μM) | 0 | −0.453 | 1.92 | −0.415 | 0.753 | 4.11 | 19.5 | 46.8 | 76.6 | 93.1 |
| | 0.2 | −1.72 | −2.19 | −4.54 | −0.305 | 10.9 | 22.6 | 52.2 | 80 | 93.5 |
| | 0.41 | 0.517 | −2.3 | 1.16 | 4.61 | 13.7 | 28.8 | 59 | 80.9 | 94.5 |
| | 0.82 | 2 | 4.16 | 6.84 | 10.8 | 22.6 | 38.1 | 61.8 | 83 | 93.8 |
| | 1.6 | 4.03 | 6.95 | 9.29 | 17.9 | 30.8 | 49.2 | 70.4 | 84.5 | 95 |
| | 3.3 | 7.51 | 12.8 | 12.9 | 25.9 | 41.2 | 60.6 | 75.2 | 86.7 | 94.6 |
| | 6.5 | 11.9 | 18.4 | 25.4 | 41.2 | 52.2 | 64.2 | 79.9 | 88.7 | 95.2 |
| | 13 | 22.2 | 36.2 | 39.6 | 47.8 | 63.2 | 74.8 | 85.1 | 89.6 | 94.6 |
| | 26 | 35.2 | 47.4 | 47.7 | 60.2 | 74 | 79.7 | 88.1 | 90.6 | 93.8 |

TABLE 95

| | | Nortriptyline HCl (μM) | | | | | | | | |
|---|---|---|---|---|---|---|---|---|---|---|
| | | 0 | 0.26 | 0.52 | 1 | 2.1 | 4.2 | 8.3 | 17 | 33 |
| Thiethylperazine Maleate | 0 | −18 | −20 | −20 | −20 | −16 | −7.2 | 22 | 67 | 89 |
| (μM) | 0.2 | −20 | −20 | −20 | −20 | −18 | −16 | 23 | 71 | 66 |
| | 0.39 | −20 | −20 | −20 | −20 | −18 | −20 | 17 | 65 | 93 |
| | 0.78 | −20 | −20 | −20 | −20 | −20 | −19 | 19 | 64 | 94 |
| | 1.6 | −20 | −20 | −20 | −20 | −20 | −20 | 24 | 72 | 94 |
| | 3.1 | −20 | −20 | −20 | −20 | −20 | −15 | 41 | 76 | 94 |
| | 6.3 | −20 | −20 | −20 | −20 | −11 | 25 | 63 | 85 | 93 |
| | 13 | 16 | 31 | 37 | 43 | 62 | 76 | 90 | 95 | 97 |
| | 25 | 87 | 89 | 88 | 91 | 92 | 94 | 96 | 96 | 97 |

TABLE 96

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Amoxapine | 0 | −4.488 | 3.79 | 2.075 | 0.588 | 6.533 | 7.025 | 9.268 | −11.88 | 0.658 |
| (μM) | 0.2 | −7.9 | 4.467 | −9.568 | −8.582 | −6.31 | −1.023 | 3.327 | −1.575 | 6.533 |
| | 0.4 | −4.195 | 2.678 | 8.508 | 10.13 | 2.303 | 20.86 | 16.2 | 17.9 | 30.96 |
| | 0.8 | −3.44 | −7.86 | −4.847 | 0.668 | 13.38 | 6.778 | 15.62 | 18.45 | 40 |
| | 1.6 | −0.878 | 3.607 | 21.83 | 13.07 | 15.03 | 19.85 | 20.1 | 27.52 | 59.78 |
| | 3.2 | 16.44 | 10.91 | 14.09 | 22.75 | 17.14 | 23.53 | 31.25 | 55 | 76.68 |
| | 6.4 | 44.67 | 38.3 | 40.88 | 36.65 | 48.73 | 44.45 | 56.78 | 74.68 | 87.82 |
| | 13 | 74.33 | 72.78 | 74.15 | 75.32 | 76.75 | 79.85 | 83.6 | 87.47 | 90.5 |
| | 25 | 89.95 | 90.3 | 90.2 | 91.12 | 90.13 | 90.35 | 90.78 | 91.53 | 91.15 |

TABLE 97

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Nortriptyline | 0 | −0.645 | −2.51 | −2.027 | −2.006 | −4.9 | −1.625 | 8.59 | 5.465 | 19.5 |
| HCl (μM) | 0.26 | 1.133 | 2.751 | −2.025 | −0.43 | −0.665 | −5.325 | 7.645 | 3.55 | 14.6 |
| | 0.52 | 0.017 | −1.603 | −0.43 | 2.515 | 1.19 | 0.965 | 1.175 | 4.364 | 17.25 |
| | 1 | 1.47 | 2.12 | 1.45 | −3.11 | 0.085 | 1.642 | −1.085 | 4.76 | 17.5 |
| | 2.1 | 6.192 | 1.231 | 2.445 | 2.005 | 0.128 | 1.935 | 2.56 | 8.25 | 26.45 |
| | 4.2 | 4.775 | 4.43 | 9.355 | 6.01 | 9.565 | 16.7 | 8.13 | 17.21 | 41.85 |
| | 8.3 | 19.2 | 21.1 | 23.6 | 24.65 | 20.3 | 25.2 | 34.5 | 54 | 72.9 |
| | 17 | 65.7 | 63.2 | 69.45 | 75.45 | 72.65 | 73 | 77.6 | 89.2 | 91.55 |
| | 33 | 93.45 | 93.1 | 85 | 93.85 | 94.35 | 93.6 | 95.75 | 96.9 | 95.2 |

TABLE 98

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Paroxetine | 0 | −10 | 1.025 | −2.7 | −5.179 | −8.74 | −1.65 | −3.5 | 0.085 | −7.435 |
| HCl (μM) | 0.21 | −5.225 | −5.469 | −5.484 | −14.5 | −20 | 2.286 | −2.8 | 3.75 | 0.45 |
| | 0.42 | −0.2 | −6.655 | −9.32 | −6.335 | 8.78 | 0.15 | −1.1 | −0.75 | 8.25 |
| | 0.83 | −3.33 | −1.22 | −11.44 | −10.29 | 0.7 | 3.5 | 2.75 | −1.67 | 22.2 |
| | 1.7 | −10.98 | −14.15 | −1.75 | −9.3 | −10.69 | 0.65 | −10.57 | 2.26 | 18.8 |
| | 3.3 | −10.29 | 3.97 | 7.91 | −0.3 | −2.5 | 5.25 | 12.55 | 27.45 | 37.6 |
| | 6.7 | 34 | 28.75 | 29.95 | 35.5 | 38.65 | 39 | 51.4 | 59.3 | 77.75 |
| | 13 | 75.8 | 75.25 | 74.95 | 68.05 | 78.5 | 81.55 | 82.25 | 85.65 | 91.25 |
| | 27 | 90.5 | 90.3 | 88.55 | 91.1 | 92.5 | 90.5 | 90.55 | 91.2 | 91.15 |

TABLE 99

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Amoxapine | 0 | −0.219 | −3.82 | 6.023 | 0.683 | 4.343 | 2.677 | 12.85 | 30.4 | 80.53 |
| (μM) | 0.2 | 9.063 | 5.803 | 1.77 | −1.553 | −4.441 | −2.33 | 13.51 | 34.33 | 78.47 |
| | 0.4 | 4.837 | 9.837 | −1.407 | 5.187 | 4.63 | 4.733 | 16.08 | 46.4 | 85.7 |
| | 0.8 | 3.623 | −0.373 | −1.067 | 5.797 | 8.207 | 9.44 | 20.83 | 49.97 | 84.7 |
| | 1.6 | 8.75 | 2.833 | 4.667 | 4.033 | 10.28 | 17.54 | 32.03 | 61.2 | 87.87 |
| | 3.2 | 25.53 | 22.33 | 22.17 | 25.87 | 25.63 | 39.6 | 51.6 | 77.5 | 88.67 |
| | 6.4 | 44.53 | 44.43 | 45.1 | 46.23 | 49.63 | 52.7 | 71.57 | 84.17 | 91.67 |
| | 13 | 70.67 | 72.5 | 71.93 | 72.9 | 82.83 | 81.7 | 80.57 | 90.23 | 93.03 |
| | 25 | 86.53 | 88.87 | 88.3 | 90.3 | 90.03 | 91.27 | 88.53 | 90.93 | 90.63 |

TABLE 100

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Fluoxetine | 0 | 1.425 | −12.39 | −14.75 | 2.35 | −0.55 | 1.65 | 7.55 | 30.8 | 69.55 |
| HCl (μM) | 0.23 | 5.6 | −13.1 | −7.28 | −1.35 | −4.45 | 0.55 | 2.5 | 27.9 | 70.25 |
| | 0.45 | −2.685 | −4.545 | −7.03 | 4.35 | 4.4 | −2.05 | 6.35 | 25.6 | 71.2 |
| | 0.9 | −3.675 | −10.42 | −0.935 | −3.15 | −0.15 | −0.95 | 4.1 | 25.6 | 70.65 |
| | 1.8 | −2.32 | −2.11 | 0.665 | 0.2 | 1.3 | −1.15 | 1.15 | 19.34 | 60.85 |
| | 3.6 | −8.62 | −3.42 | −4.405 | −0.15 | −3.45 | −4.65 | 4.8 | 31.25 | 67.95 |
| | 7.2 | 0.3 | −3.3 | −0.15 | 1.35 | 0.45 | −7.89 | 0.45 | 28.4 | 76.2 |
| | 14 | −11.41 | −0.885 | −10.32 | −3.15 | 0.2 | 0.95 | 4.65 | 33 | 76.4 |
| | 29 | 1.35 | 0.65 | 3.5 | −1.6 | 0.55 | −2.75 | 5.15 | 50.5 | 78.65 |

TABLE 101

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| NOrtriptyline | 0 | 3.19 | −5.179 | −0.745 | 2.43 | 0.5433 | −4.483 | 13.5 | 31 | 69.37 |
| HCl (μM) | 0.26 | −3.427 | 1.947 | 3.297 | 5.59 | 11.5 | 12.27 | 15.47 | 40.17 | 67.33 |
| | 0.52 | 5.453 | 5.597 | 4.697 | 5.06 | 16.13 | 11.94 | 18.63 | 44.43 | 77.1 |
| | 1 | 8.54 | 5.79 | 7.547 | 4.87 | 14.4 | 14.17 | 22.53 | 47.77 | 78.83 |
| | 2.1 | 12.58 | 11.43 | 9.41 | 10.45 | 16.03 | 21 | 30.53 | 52.47 | 82.23 |
| | 4.2 | 21.43 | 17.97 | 18.63 | 21.6 | 28.33 | 31.87 | 40.97 | 62.13 | 74.07 |
| | 8.3 | 37.77 | 44.83 | 45.67 | 50.83 | 59.1 | 58.57 | 63.27 | 76.27 | 87.7 |
| | 17 | 76.93 | 80.73 | 71.87 | 78.2 | 79.77 | 81.1 | 81.47 | 86 | 86.17 |
| | 33 | 87.87 | 89.3 | 87.67 | 90.17 | 90.63 | 89.57 | 89.17 | 89.3 | 87.2 |

TABLE 102

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Paroxetine | 0 | 1.515 | −7.175 | 0.97 | −1.54 | −3.655 | −3.4 | −0.7 | 36.7 | 78.85 |
| HCl (μM) | 0.21 | 1.055 | −3.004 | −2.135 | −0.235 | 1.307 | −4.9 | −1.15 | 39.75 | 79.8 |
| | 0.42 | −4.1 | 1.905 | 3.509 | −0.225 | −3.635 | 4.125 | 11.18 | 47.45 | 81.2 |
| | 0.83 | −0.81 | −2.151 | 2.564 | −1.352 | 2.999 | 6.92 | 10.24 | 41.35 | 81.5 |
| | 1.7 | 1.295 | 0.005 | 5.515 | 5.115 | 9.37 | 16.93 | 24.8 | 53.4 | 83 |
| | 3.3 | 20.7 | 24.85 | 23.05 | 26.5 | 30.15 | 29.75 | 38.45 | 74.9 | 87.75 |
| | 6.7 | 54.1 | 47.05 | 55.55 | 56.4 | 56.2 | 59.05 | 65.25 | 82.55 | 87.25 |
| | 13 | 78.1 | 82.1 | 80 | 83.6 | 82.9 | 87.9 | 89.15 | 92.65 | 94.8 |
| | 27 | 94.25 | 94.5 | 90.95 | 95.2 | 95 | 94.5 | 95 | 95.35 | 92.7 |

TABLE 103

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Fluoxetine | 0 | −2.092 | 0 | 0.555 | −9.315 | −5.25 | −2.08 | 9.81 | 9.155 | 9.27 |
| HCl (μM) | 0.23 | 6.903 | −0.075 | 2.67 | −0.475 | 0 | −5.895 | 3.453 | 0.335 | 18.35 |
| | 0.45 | 1.66 | 4.221 | 9.185 | 11.4 | 3.425 | −5.165 | 3.81 | −2.755 | 14.7 |
| | 0.9 | 4.76 | 4.436 | −0.76 | 6.288 | 1.12 | −4.92 | −10.14 | −4.515 | 16 |
| | 1.8 | 0.37 | 5.766 | −7.465 | 3.805 | 1.73 | −1.539 | −0.695 | 0.6 | 19.15 |
| | 3.6 | 4.441 | −2.17 | 0.165 | 11.06 | 14.05 | 4.41 | −4.04 | 8.59 | 18.8 |
| | 7.2 | 16.45 | 15.75 | 22.05 | 5.69 | 12.65 | 19.55 | 10.02 | 17.85 | 33.45 |
| | 14 | 50.05 | 44 | 41.05 | 44.6 | 44.7 | 44.5 | 46.2 | 51.9 | 66.25 |
| | 29 | 42.25 | 43.85 | 41.05 | 77.9 | 80.15 | 80 | 80.45 | 82.8 | 85.25 |

TABLE 104

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Fluoxetine | 0 | 0.355 | −0.684 | −1.09 | −3.095 | −1.243 | 3.415 | 12.8 | 51.78 | 72.45 |
| HCl (μM) | 0.23 | 1.263 | −1.548 | −1.845 | −0.238 | 1.332 | 1.908 | 13.12 | 62 | 89.6 |
| | 0.45 | 2.848 | 1.266 | −0.573 | 0.2075 | −2.062 | 1.41 | 15.55 | 56.3 | 84.62 |
| | 0.9 | −1.037 | −2.988 | −4.63 | −1.35 | −0.01 | 0.923 | 17.73 | 62.73 | 92.35 |
| | 1.8 | −3.552 | −1.749 | −3.335 | −5.243 | −0.325 | 4.475 | 21.13 | 66 | 90.97 |
| | 3.6 | −4.435 | −6.622 | −6.302 | −5.43 | 1.415 | 7.107 | 29.02 | 69.45 | 90.85 |
| | 7.2 | 4.83 | 5.452 | 5.393 | 4.377 | 8.858 | 20.33 | 42.78 | 75.55 | 90.78 |
| | 14 | 37.42 | 38.55 | 38.47 | 33.3 | 51.2 | 53.4 | 69.53 | 82 | 90.55 |
| | 29 | 75.85 | 73.53 | 81.05 | 77.95 | 80.65 | 80.83 | 86.5 | 87.3 | 87.2 |

TABLE 105

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Fluoxetine | 0 | −0.242 | 0 | −2.482 | −1.02 | −2.779 | −2.203 | 3.945 | 22 | 62.3 |
| HCl (μM) | 0.23 | 2.417 | −0.778 | −0.548 | −2.038 | −3.393 | −0.598 | 0.37 | 19.34 | 64.97 |
| | 0.45 | 2.796 | −1.95 | 1.485 | 1.82 | −1.102 | 1.467 | 4.372 | 20.27 | 63.1 |
| | 0.9 | 2.775 | 1.323 | 1.693 | −2.495 | −1.59 | −2.363 | 0.6925 | 17.76 | 62.83 |
| | 1.8 | −0.649 | 0.3178 | −2.338 | −1.462 | −2.502 | 1.22 | 8.59 | 21.2 | 71.5 |
| | 3.6 | 2.808 | 0.0125 | 0.6375 | −2.1 | −2.77 | 4.742 | 13.35 | 26.67 | 74.55 |
| | 7.2 | 6.935 | 13.19 | 9.62 | 10.61 | 10.13 | 15.21 | 25.82 | 46.95 | 72.85 |
| | 14 | 48.27 | 44.57 | 47.15 | 45.77 | 45.05 | 49.6 | 58.35 | 64.05 | 83.35 |
| | 29 | 62.67 | 77.53 | 79.93 | 79.8 | 81.62 | 81.3 | 84.62 | 86.83 | 85.9 |

TABLE 106

| | | Thiethylperazine Maleate (μM) | | | | | | | | |
|---|---|---|---|---|---|---|---|---|---|---|
| | | 0 | 0.2 | 0.39 | 0.78 | 1.6 | 3.1 | 6.3 | 13 | 25 |
| Fluoxetine | 0 | 2.747 | −2.477 | −3.396 | 2.645 | 2.277 | 2.86 | 4.95 | 25.15 | 80.97 |
| HCl (μM) | 0.23 | 1.78 | 3.225 | −2.083 | −1.222 | −2.335 | −3.33 | 2.545 | 33.65 | 81.5 |
| | 0.45 | 6.095 | 0.008 | −1.67 | 1.616 | 0.662 | 1.493 | 7.032 | 38.38 | 81.17 |
| | 0.9 | 0.158 | 2.045 | −0.465 | 2.407 | −1.022 | 10.28 | 13.89 | 42.58 | 82.4 |
| | 1.8 | 2.204 | 1.512 | 5.213 | 2.521 | −0.173 | 9.15 | 6.815 | 35.25 | 79.25 |
| | 3.6 | 9.982 | 7.078 | 9.21 | 4.636 | 2.264 | 6.923 | 22.53 | 47.92 | 81.75 |
| | 7.2 | 12.92 | 15.03 | 14.31 | 17.78 | 24.41 | 26.2 | 36 | 58.08 | 87.88 |
| | 14 | 43.98 | 47.15 | 48.92 | 48.8 | 50.15 | 53.9 | 62.55 | 77.35 | 86.53 |
| | 29 | 43.98 | 47.15 | 47.38 | 48.8 | 50.15 | 50.95 | 58.77 | 77.35 | 78.02 |

TABLE 107

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Desipramine | 0 | −1.175 | −4.625 | −2.911 | −3.667 | −4.275 | −7.91 | −4.35 | 3.185 | 14.95 |
| HCl (μM) | 0.25 | 0.985 | −1.929 | −3.811 | −2.05 | −2.06 | −2.455 | −1.23 | 2.9 | 18.2 |
| | 0.5 | 0.14 | −0.561 | 1.563 | −0.85 | −0.395 | −3.355 | −4.4 | 1.45 | 16.38 |
| | 1 | −0.517 | −0.284 | 2.445 | 0.105 | −2.44 | −2.26 | −4.42 | 1.835 | 18.38 |
| | 2 | −2.36 | −6.98 | −4.605 | −4.616 | −4.735 | −4.76 | −5.195 | −0.235 | 12.12 |
| | 4 | −5.155 | −1.32 | −4.78 | −4.354 | −2.1 | −4.955 | 0.275 | 6 | 25.7 |
| | 8 | 6.04 | 5.9 | 7.415 | 9.87 | 9.275 | 6.845 | 15.74 | 24.7 | 48.25 |
| | 16 | 38.25 | 32.65 | 33.1 | 39.3 | 35.6 | 40.05 | 41.55 | 63.05 | 77.5 |
| | 32 | 84.6 | 81.4 | 80.75 | 81.65 | 86.2 | 83.75 | 85.1 | 89.15 | 94.95 |

TABLE 108

| | | Clemizole HCl (μM) | | | | | | | | |
|---|---|---|---|---|---|---|---|---|---|---|
| | | 0 | 0.22 | 0.43 | 0.86 | 1.7 | 3.5 | 6.9 | 14 | 28 |
| Sertraline | 0 | −0.71 | −1.122 | −2.165 | 1.44 | 3.59 | 0.47 | 2.25 | 4.65 | 16.94 |
| HCl (μM) | 0.25 | 0.995 | −0.855 | 2.715 | −0.035 | 2.7 | −3.8 | 2.8 | 2.8 | 19.7 |
| | 0.49 | 2.01 | 2.46 | 6.44 | −3.5 | −1.44 | 2.885 | −1.3 | 2.65 | 18.34 |
| | 0.99 | 1.525 | 1.445 | 3.61 | 1.58 | 0.465 | −3.32 | −2.35 | 4.75 | 23.41 |
| | 2 | 2.085 | 4.247 | 1.23 | 1.44 | 1.01 | 1.25 | 0.1 | 9.24 | 28.55 |
| | 4 | 12.84 | 7.976 | 11.55 | 13 | 8.945 | 18.3 | 17.12 | 29.75 | 41.2 |
| | 7.9 | 44 | 41.2 | 44 | 41 | 50.65 | 51.9 | 54.45 | 64.75 | 74.15 |
| | 16 | 81.9 | 81.05 | 81.2 | 84.3 | 85.35 | 85.35 | 84.55 | 87.35 | 88.85 |
| | 32 | 87.8 | 78.25 | 85.45 | 91.65 | 92.15 | 87.75 | 87.2 | 91.15 | 88.8 |

TABLE 109

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Desipramine | 0 | 1.296 | −1.9 | 1.635 | −1.67 | 0.863 | 2.932 | 1705 | 62.98 | 95.22 |
| HCl (μM) | 0.25 | 2.855 | 0.165 | −2.078 | −0.650 | 0.113 | 4.215 | 16.07 | 71.53 | 95.12 |
| | 0.5 | 0.698 | 0.656 | 0.972 | −0.308 | 0.923 | 3.312 | 21.32 | 69.3 | 95.47 |
| | 1 | 0.028 | −2.205 | 2.19 | −0.21 | 0.638 | 6.835 | 20.46 | 74.58 | 95.33 |
| | 2 | −3.235 | −0.818 | 1.521 | −0.765 | 0.685 | 2.788 | 23.03 | 67.58 | 92.9 |
| | 4 | −1.81 | −2.729 | 0.289 | 2.261 | 5.864 | 7.688 | 29.23 | 77.38 | 95.22 |
| | 8 | 11.9 | 14.77 | 12.17 | 12.15 | 15.17 | 26.88 | 48.52 | 85.45 | 93.92 |
| | 16 | 33.55 | 35.5 | 42.9 | 40.42 | 48.1 | 53.67 | 72.9 | 89.33 | 92.28 |
| | 32 | 77.7 | 80.72 | 80.88 | 79.62 | 84.4 | 82.7 | 89 | 91.38 | 88.45 |

TABLE 110

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Sertraline | 0 | −2.15 | −4.173 | −4.062 | −7.319 | −4.985 | 2.095 | 14.15 | 44.97 | 84.03 |
| HCl (μM) | 0.25 | −0.64 | −5.798 | −8.12 | −5.43 | −6.361 | 1.047 | 13.53 | 59.35 | 80.55 |
| | 0.49 | −3.183 | 0.45 | −9.15 | −7.503 | 3.895 | 3.16 | 15.47 | 58.35 | 86.4 |
| | 0.99 | −5.712 | −0.34 | −7.567 | −1.265 | −0.363 | −2.877 | 29.47 | 66.3 | 86.15 |
| | 2 | −3.712 | −7.227 | −5.218 | 2.61 | 3.762 | 13.55 | 45.1 | 63.4 | 81.02 |
| | 4 | 10.8 | 4.275 | 3.018 | 2.925 | 10.38 | 25.62 | 46.08 | 65.92 | 82.17 |
| | 7.9 | 40.05 | 36.15 | 37.25 | 44.77 | 41.38 | 56.75 | 66.88 | 76.5 | 85.42 |
| | 16 | 73.25 | 75.53 | 75.23 | 73.58 | 76.7 | 75.9 | 77.65 | 81.88 | 84.58 |
| | 32 | 80.05 | 83.58 | 86.47 | 84.03 | 83.72 | 79.95 | 76.15 | 79.38 | 80.47 |

TABLE 111

| | Promethazine HCl (μM) | | | | | | | | | |
|---|---|---|---|---|---|---|---|---|---|---|
| | | 0 | 0.24 | 0.49 | 0.97 | 1.9 | 3.9 | 7.8 | 16 | 31 |
| Desipramine HCl | 0 | −0.14 | 0 | 5.92 | 4.54 | 7.95 | 7.95 | 39.4 | 95.7 | 78.8 |
| (μM) | 0.25 | 6.72 | 10.3 | 10.3 | 10.3 | 8.15 | 12.3 | 31.3 | 95.9 | 83.6 |
| | 0.5 | 6.72 | 5.79 | 5.79 | 5.79 | 7.94 | 26.5 | 31.3 | 95.9 | 84.6 |
| | 1 | 6.72 | 1.49 | 17.8 | 9.64 | 10 | 26.5 | 31.3 | 93.3 | 86.2 |
| | 2 | 6.72 | 1.49 | 4.87 | 5.98 | 17.4 | 26.5 | 31.3 | 91.9 | 89.7 |
| | 4 | 9.65 | 2.29 | 14 | 13.9 | 22 | 26.5 | 31.3 | 91.9 | 96.1 |
| | 8 | 19.9 | 21.4 | 49.5 | 37.9 | 33.1 | 39.9 | 88.1 | 84.7 | 89.3 |
| | 16 | 43 | 45.1 | 58.1 | 45.9 | 54.8 | 66.1 | 88.1 | 84.7 | 90.6 |
| | 32 | 85.3 | 83.5 | 86.6 | 83.1 | 79.6 | 85.9 | 89 | 89.1 | 90.9 |

TABLE 112

| | | Promethazine HCl (μM) | | | | | | | | |
|---|---|---|---|---|---|---|---|---|---|---|
| | | 0 | 0.24 | 0.49 | 0.97 | 1.9 | 3.9 | 7.8 | 16 | 31 |
| Sertraline | 0 | 1.79 | −1.59 | −1.715 | −1.665 | −5.565 | −4.745 | −3.02 | 22.65 | 73.5 |
| HCl (μM) | 0.25 | −0.047 | −2.423 | −4.885 | −7.35 | −3.92 | −8.395 | −5.905 | 18.92 | 68.95 |
| | 0.49 | −3.07 | 3.535 | −3.89 | −4.41 | −6.335 | −9.162 | −6.015 | 22.4 | 70.6 |
| | 0.99 | −2.27 | −6.721 | −5.384 | −7.3 | −11.15 | −4.88 | −5 | 26.35 | 75.15 |
| | 2 | 0.785 | 0.215 | −4.385 | −2.995 | −3.855 | −1.9 | 6.445 | 32.2 | 76.65 |
| | 4 | 10.21 | 10.25 | 4.532 | 1.59 | 5.39 | 7.506 | 18.75 | 54.25 | 81.2 |
| | 7.9 | 44.05 | 43.1 | 47.05 | 40.05 | 53.15 | 52.05 | 68 | 83.55 | 93 |
| | 16 | 87.5 | 80.55 | 82.3 | 87.6 | 89.45 | 89.2 | 89.55 | 94.45 | 95.9 |
| | 32 | 95.15 | 96.3 | 96.1 | 94.3 | 96.5 | 96.3 | 96.6 | 87.1 | 96.05 |

TABLE 113

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Amoxapine | 0 | 0 | −0.797 | 0.926 | −1.27 | −0.1 | 8.7 | 23 | 74 | 83.6 |
| (μM) | 0.2 | 5.52 | 5.87 | 2.42 | 0.49 | 1.88 | 7.17 | 24.6 | 71.4 | 96.1 |
| | 0.4 | 6.51 | 7.68 | 8.25 | 0.125 | 5.05 | 12.5 | 23.3 | 74.5 | 96.1 |
| | 0.8 | 6.08 | 6.12 | 7.67 | 4.05 | 3.75 | 20.1 | 29.1 | 73.9 | 96.2 |
| | 1.6 | 9.59 | 14.6 | 12.3 | 4.77 | 11.8 | 16.5 | 38.3 | 73.2 | 95.8 |
| | 3.2 | 18.9 | 20.5 | 20.8 | 14.1 | 16 | 30.5 | 42.1 | 78.7 | 95.8 |
| | 6.4 | 40.8 | 39.8 | 39.2 | 37.6 | 33.9 | 47.2 | 65.5 | 92.2 | 97 |
| | 13 | 63.3 | 59.5 | 61 | 63.1 | 64.3 | 71.2 | 78.8 | 95.9 | 96.7 |
| | 25 | 85.3 | 88.7 | 87.3 | 87.8 | 93.3 | 90.5 | 92.7 | 96.9 | 96.9 |

TABLE 114

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Nortriptyline | 0 | −0.763 | −12.9 | −3.59 | −10.1 | −0.63 | 10.4 | 28 | 67.4 | 81 |
| HCl (μM) | 0.26 | 4.34 | 15 | 4.46 | 1.1 | 5.2 | 15.2 | 30.6 | 74.2 | 84.5 |
| | 0.52 | 9.94 | 8.49 | 5.63 | 14.4 | 13.5 | 14.2 | 33.9 | 80.3 | 88.9 |
| | 1 | 12.6 | 5.49 | 9.11 | 9.3 | 14.3 | 14.8 | 33.4 | 77.6 | 85.9 |
| | 2.1 | 11.8 | 12 | 8.15 | 9.35 | 8.13 | 21.4 | 47.1 | 82.2 | 92.4 |
| | 4.2 | 9.62 | 15.8 | 13.8 | 16.2 | 18.6 | 32.7 | 56.4 | 86.8 | 89.5 |
| | 8.3 | 38 | 40.2 | 41 | 46.8 | 48 | 64 | 76.1 | 89.1 | 93.5 |
| | 17 | 72.1 | 78.9 | 78.6 | 81.7 | 81.6 | 82.9 | 85.5 | 91.4 | 95.1 |
| | 33 | 89.8 | 93.5 | 94.9 | 90.3 | 92 | 93 | 92.7 | 94.6 | 92.1 |

TABLE 115

| | | Desloratadine (μM) | | | | | | | | |
|---|---|---|---|---|---|---|---|---|---|---|
| | | 0 | 0.25 | 0.51 | 1 | 2 | 4.1 | 8.1 | 16 | 33 |
| Paroxetine HCl | 0 | 4.85 | 0.342 | −2.85 | 0.205 | 2.14 | 12.9 | 30.2 | 67.2 | 84.3 |
| (μM) | 0.21 | 0.569 | −3.83 | −3.43 | −2.31 | 3.5 | 13.8 | 32.8 | 69.4 | 86.6 |
| | 0.42 | −5.5 | −5.41 | −0.51 | 2.11 | 6.27 | 18.6 | 36.6 | 77.8 | 86.7 |
| | 0.83 | 0.357 | −2.04 | −5.42 | −4.63 | 4.02 | 6.5 | 34.3 | 75.1 | 88.1 |
| | 1.7 | −2.54 | 2.63 | −1.19 | 0.785 | 8.56 | 13.5 | 53 | 77 | 85.3 |
| | 3.3 | 13 | 12.4 | 5.76 | 24.9 | 20.2 | 23.9 | 60 | 82.8 | 87.5 |
| | 6.7 | 36.4 | 29.2 | 36.3 | 40.3 | 51.9 | 58.4 | 73.6 | 89.4 | 88.1 |
| | 13 | 68 | 64.2 | 72 | 73.4 | 74.6 | 81.5 | 89.3 | 90.2 | 86.3 |
| | 27 | 85.5 | 88.7 | 87.6 | 85.8 | 86.6 | 86.9 | 88.8 | 86.8 | 85.2 |

### Other Embodiments

Various modifications and variations of the described method and system of the invention will be apparent to those skilled in the art without departing from the scope and spiri of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification. are herein incorporated by reference to the 'same extent as if each independent publication was specifically and individually incorporated by reference.

1. A composition comprising an antihistamine or an antihistamine analog and a corticosteroid.

2. The composition of claim 1, wherein said antihistamine is bromodiphenhydramine, clemizole, cyproheptadine, desloratadine, loratadine, thiethylperazine maleate, epinastine, or promethazine.

3. The composition of claim 1, wherein said corticosteroid is prednisolone, cortisone, dexamethasone, hydrocortisone, methylprednisolone, fluticasone, prednisone, triamcinolone, or diflorasone.

4. The composition of claim 1, wherein said antihistamine is desloratadine or loratadine and said corticosteroid is prednisolone.

5. The composition of claim 1, wherein said composition is formulated for topical administration.

6. The composition of claim 1, wherein said composition is formulated for systemic administration.

7. The composition of claim 1, wherein said antihistamine or analog thereof or said corticosteroid is present in said composition in a low dosage.

8. The composition of claim 1, wherein said antihistamine or analog thereof or said corticosteroid is present in said composition in a high dosage.

9. The composition of claim 1, wherein said composition further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid.

10. The composition of claim 9, wherein said NSAID is ibuprofen, diclofenac, or naproxen.

11. The composition of claim 9, wherein said COX-2 inhibitor is rofecoxib, celecoxib, valdecoxib, or lumiracoxib.

12. The composition of claim 9, wherein said biologic is adelimumab, etanercept, or infliximab.

13. The composition of claim 9, wherein said DMARD is methotrexate or leflunomide.

14. The composition of claim 9, wherein said xanthine is theophylline.

15. The composition of claim 9, wherein said anticholinergic compound is ipratropium or tiotropium.

16. The composition of claim 9, wherein said beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline.

17. The composition of claim 9, wherein said vitamin D analog is calcipotriene or calcipotriol.

18. The composition of claim 9, wherein said psoralen is methoxsalen.

19. The composition of claim 9, wherein said retinoid is acitretin or tazoretene.

20. The composition of claim 9, wherein said 5-amino salicylic acid is mesalamine, sulfasalazine, balsalazide disodium, or olsalazine sodium.

21. The composition of claim 9, wherein said small molecule immunomodulator is VX 702, SCIO 469, doramapimod, RO 30201195, SCIO 323, DPC 333, pranalcasan, mycophenolate, or merimepodib.

22. A method for treating a patient diagnosed with or at risk of developing an immunoinflammatory disorder, said method comprising administering to the patient an antihistamine or analog thereof and a corticosteroid simultaneously or within 14 days of each other in amounts sufficient to treat said patient

23. The method of claim 22, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

24. The method of claim 22, wherein said antihistamine or analog thereof and said corticosteroid are administered simultaneously.

25. A kit comprising:
(i) a composition comprising an antihistamine or an analog thereof and a corticosteroid; and
(ii) instructions for administering said composition to a patient diagnosed with or at risk of developing an immunoinflammatory disorder.

26. A kit comprising:
(i) an antihistamine or an analog thereof;
(ii) a corticosteroid; and
(iii) instructions for administering said antihistamine and said corticosteroid to a patient diagnosed with or at risk of developing an immunoinflammatory disorder.

27. A kit comprising:
(i) an antihistamine or an analog thereof; and
(ii) instructions for administering said antihistamine or analog thereof and a corticosteroid to a patient diagnosed with or at risk of developing an immunoinflammatory disorder.

28. A kit comprising:
(i) a corticosteroid; and
(ii) instructions for administering said corticosteroid and an antihistamine or an analog thereof to a patient diagnosed with or at risk of developing an immunoinflammatory disorder.

29. A composition comprising an antihistamine or an antihistamine analog and ibudilast or an analog thereof.

30. The composition of claim 29, wherein said antihistamine is bromodiphenhydramine, clemizole, cyproheptadine, desloratadine, ioratadine, thiethylperazine maleate, epinastine, or promethazine.

31. The composition of claim 29, said composition comprising (i) desloratadine or loratadine and (ii) ibudilast.

32. The composition of claim 29, wherein said composition is formulated for topical administration.

33. The composition of claim 29, wherein said composition is formulated for systemic administration.

34. The composition of claim 29, wherein said antihistamine or analog thereof or said ibudilast or analog thereof is present in said composition in a low dosage.

35. The composition of claim 29, wherein said antihistamine or analog thereof or said ibudilast or analog thereof is present in said composition in a high dosage.

36. The composition of claim 29, wherein said composition further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

37. A method for treating a patient diagnosed with or at risk of developing an immunoinflammatory disorder, said method comprising administering to the patient an antihistamine or analog thereof and ibudilast or an analog thereof simultaneously or within 14 days of each other in amounts sufficient to treat said patient

38. The method of claim 37, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

39. The method of claim 56, wherein said antihistamine or analog thereof and said ibudilast or analog thereof are administered simultaneously.

40. A kit comprising:
(i) a composition comprising an antihistamine or an analog thereof and ibudilast or analog thereof; and
(ii) instructions for administering said composition to a patient diagnosed with or at risk of developing an immunoinflammatory disorder.

41. A kit comprising:
(i) an antihistamine or an analog thereof;
(ii) ibudilast or analog thereof; and
(iii) instructions for administering said antihistamine or analog thereof and said ibudilast or analog thereof to a patient diagnosed with or at risk of developing an immunoinflammatory disorder.

42. A kit comprising:
(i) an antihistamine or an analog thereof; and
(ii) instructions for administering said antihistamine or analog thereof and ibudilast or analog thereof to a patient diagnosed with or at risk of developing an immunoinflammatory disorder.

43. A kit comprising:
(i) ibudilast or an analog thereof; and
(ii) instructions for administering said ibudilast or analog thereof and an antihistamine or analog thereof to a patient diagnosed with or at risk of developing an immunoinflammatory disorder.

44. A composition comprising an antihistamine or an antihistamine analog and rolipram or an analog thereof.

45. The composition of claim 44, wherein said antihistamine is bromodiphenhydramine, clemizole, cyproheptadine, desloratadine, loratadine, thiethylperazine maleate, epinastine, or promethazine.

46. The composition of claim 45, said composition comprising desloratadine or loratadine and rolipram.

47. A method for treating a patient diagnosed with or at risk of developing an immunoinflammatory disorder, said method comprising administering to the patient an antihistamine or analog thereof and rolipram or an analog thereof simultaneously or within 14 days of each other in amounts sufficient to treat said patient

48. The method of claim 47, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

49. The method of claim 47, wherein said antihistamine or analog thereof and said rolipram or analog thereof are administered simultaneously.

50. A kit comprising:
(i) a composition comprising an antihistamine or an analog thereof and rolipram or an analog thereof; and
(ii) instructions for administering said composition to a patient diagnosed with or at risk of developing an immunoinflammatory disorder.

51. A kit comprising:
(i) an antihistamine or an analog thereof;
(ii) rolipram or an analog thereof; and
(iii) instructions for administering said antihistamine or analog thereof and said rolipram or analog thereof to a patient diagnosed with or at risk of developing an immunoinflammatory disorder.

52. A kit comprising:
(i) an antihistamine or an analog thereof; and
(ii) instructions for administering said antihistamine or analog thereof and rolipram or an analog thereof to a patient diagnosed with or at risk of developing an immunoinflammatory disorder.

53. A kit comprising:
(i) rolipram or an analog thereof; and
(ii) instructions for administering said rolipram or analog thereof and an antihistamine or analog thereof to a patient diagnosed with or at risk of developing an immunoinflammatory disorder.

54. A composition comprising an antihistamine or an antihistamine analog and a tetra-substituted pyrimidopyrimidine.

55. The composition of claim 91, wherein said antihistamine is bromodiphenhydramine, clemizole, cyproheptadine, desloratadine, loratadine, thiethylperazine maleate, epinastine, or promethazine.

56. The composition of claim 55, wherein said tetra-substituted pyrimidopyrimidine is dipyridimole.

57. The composition of claim 55, said antihistamine is desloratadine or loratadine and said tetra-substituted pyrimidopyrimidine is dipyridimole.

58. The composition of claim 55, wherein said composition is formulated for topical administration.

59. The composition of claim 55, wherein said composition is formulated for systemic administration.

60. The composition of claim 55, wherein said antihistamine or analog thereof or tetra-substituted pyrimidopyrimidine is present in said composition in a low dosage.

61. The composition of claim 55, wherein said antihistamine or analog thereof or said tetra-substituted pyrimidopyrimidine is present in said composition in a high dosage.

62. The composition of claim 55, wherein said composition further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

63. A method for treating a patient diagnosed with or at risk of developing an immunoinflammatory disorder, said method comprising administering to the patient an antihistamine or analog thereof and tetra-substituted pyrimidopyrimidine simultaneously or within 14 days of each other in amounts sufficient to treat said patient

64. The method of claim 63, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic, dermatitis, multiple sclerosis, myasthenia gravis, psoriasis, ankylosing spondylitis, cirrhosis, or psoriatic arthritis.

65. The method of claim 63, wherein said antihistamine or analog thereof and said tetra-substituted pyrimidopyrimidine are administered simultaneously.

66. A kit comprising:
(i) a composition comprising an antihistamine or an analog thereof and a tetra-substituted pyrimidopyrimidine; and
(ii) instructions for administering said composition to a patient diagnosed with or at risk of developing an immunoinflammatory disorder.

67. A kit comprising:
(i) an antihistamine or an analog thereof;
(ii) a tetra-substituted pyrimidopyrimidine; and
(iii) instructions for administering said antihistamine or analog thereof and said tetra-substituted pyrimidopyrimidine to a patient diagnosed with or at risk of developing an immunoinflammatory disorder.

68. A kit comprising:
(i) an antihistamine or an analog thereof; and
(ii) instructions for administering said antihistamine or analog thereof and a tetra-substituted pyrimidopyrimidine to a patient diagnosed with or at risk of developing an immunoinflammatory disorder.

69. A kit comprising:
(i) a tetra-substituted pyrimidopyrimidine; and
(ii) instructions for administering said tetra-substituted pyrimidopyrimidine and an antihistamine or an analog thereof to a patient diagnosed with or at risk of developing an immunoinflammatory disorder.

70. A composition comprising an antihistamine or an antihistamine analog and a tricyclic or tetracyclic antidepressant or analog thereof.

71. The composition of claim 70, wherein said antihistamine is bromodiphenhydramine, clemizole, cyproheptadine, desloratadine, loratadine, thiethylperazine maleate, epinastine, or promethazine.

72. The composition of claim 70, wherein said tricyclic antidepressant is nortryptiline, amoxapine, or desipramine.

73. The composition of claim 70, wherein said composition is formulated for topical administration.

74. The composition of claim 70, wherein said composition is formulated for systemic administration.

75. The composition of claim 70, wherein said antihistamine or analog thereof or said tricyclic or tetracyclic antidepressant or analog thereof is present in said composition in a low dosage.

76. The composition of claim 70, wherein said antihistamine or analog thereof or said tricyclic or tetracyclic antidepressant or analog thereof is present in said composition in a high dosage.

77. The composition of claim 70, wherein said composition further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

78. A method for treating a patient diagnosed with or at risk of developing an immunoinflammatory disorder, said method comprising administering to the patient an antihistamine or analog thereof and a tricyclic or tetracyclic antidepressant or analog thereof simultaneously or within 14 days of each other in amounts sufficient to treat said patient.

79. The method of claim 78, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

80. The method of claim 78, wherein said antihistamine or analog thereof and said tricyclic or tetracyclic antidepressant or analog thereof are administered simultaneously.

81. A kit comprising:
(i) a composition comprising an antihistamine or an analog thereof and a tricyclic or tetracyclic antidepressant or an analog thereof; and
(ii) instructions for administering said composition to a patient diagnosed with or at risk of developing an immunoinflammatory disorder.

82. A kit comprising:
(i) an antihistamine or an analog thereof;
(ii) a tricyclic or tetracyclic antidepressant or an analog thereof; and
(iii) instructions for administering said antihistamine or analog thereof and said tricyclic or tetracyclic antidepressant to a patient diagnosed with or at risk of developing an immunoinflammatory disorder.

83. A kit comprising:
(i) an antihistamine or an analog thereof; and
(ii) instructions for administering said antihistamine or analog thereof and a tricyclic or tetracyclic antidepressant to a patient diagnosed with or at risk of developing an immunoinflammatory disorder.

84. A kit comprising:
(i) a tricyclic or tetracyclic antidepressant; and
(ii) instructions for administering said tricyclic or tetracyclic antidepressant and an antihistamine or an analog thereof to a patient diagnosed with or at risk of developing an immunoinflammatory disorder.

85. A pharmaceutical composition comprising an antihistamine or an antihistamine analog and an SSRI or analog thereof.

86. The composition of claim 85, wherein said antihistamine is bromodiphenhydramine, clemizole, cyproheptadine, desloratadine, loratadine, thiethylperazine maleate, epinastine, or promethazine.

87. The composition of claim 85, wherein said SSRI is paroxetine or fluoxetine.

88. The composition of claim 85, wherein said composition is formulated for topical adininistration.

89. The composition of claim 85, wherein said composition is formulated for systemic administration.

90. The composition of claim 85, wherein said antihistamine or analog thereof or said SSRI or analog thereof is present in said composition in a low dosage.

91. The composition of claim 85, wherein said antihistamine or analog thereof or said SSRI or analog thereof is present in said composition in a high dosage.

92. The composition of claim 85, wherein said composition further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid.

93. A method for treating a patient diagnosed with or at risk of developing an immunoinflammatory disorder, said method comprising administering to the patient an antihistamine or analog thereof and an SSRI or analog thereof simultaneously or within 14 days of each other in amounts sufficient to treat said patient

94. The method of claim 93, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

95. The method of claim 93, wherein said antihistamine or analog thereof and said SSRI or analog thereof are administered simultaneously.

96. A kit comprising:
(i) a composition comprising an antihistamine or an analog thereof and an SSRI or an analog thereof; and
(ii) instructions for administering said composition to a patient diagnosed with or at risk of developing an immunoinflammatory disorder.

97. A kit comprising:
(i) an antihistamine or an analog thereof;
(ii) an SSRI or an analog thereof; and
(iii) instructions for administering said antihistamine or analog thereof and said SSRI or an analog thereof to a patient diagnosed with or at risk of developing an immunoinflammatory disorder.

98. A kit comprising:
(i) an antihistamine or an analog thereof; and
(ii) instructions for administering said antihistamine or analog thereof and an SSRI or an analog thereof to a patient diagnosed with or at risk of developing an immunoinflammatory disorder.

99. A kit comprising:
(i) an SSRI or an analog thereof; and
(ii) instructions for administering said SSRI or analog thereof and an antihistamine or an analog thereof to a patient diagnosed with or at risk of developing an immunoinflammatory disorder.
